(19) United States
(12) Patent Application Publication
(10) Pub. No.: US 2023/0147993 A1
(43) Pub. Date: May 11, 2023

(54) THIOPHENE COMPOUNDS WITH CYCLIC AMIDES, AND USES THEREOF

(71) Applicant: Capulus Therapeutics, LLC, San Francisco, CA (US)

(72) Inventors: Michael John GREEN, Half Moon Bay, CA (US); Barry Patrick HART, Palo Alto, CA (US)

(21) Appl. No.: 17/743,234

(22) Filed: May 12, 2022

Related U.S. Application Data
(63) Continuation of application No. PCT/US2020/060277, filed on Nov. 12, 2020.
(60) Provisional application No. 62/935,025, filed on Nov. 13, 2019.
(60) Provisional application No. 63/056,405, filed on Jul. 24, 2020.

(51) Int. Cl. C07D 409/14; C07D 491/056; C07D 498/04; C07D 471/04; C07D 493/04

(52) U.S. Cl. CPC C07D 409/14; C07D 491/056; C07D 498/04; C07D 471/04; C07D 493/04

(57) ABSTRACT

Provided herein are compounds comprising a four-ring core, such as compounds of Formula (I) and Formula (II), and pharmaceutically acceptable salts, solvates, tautomers, isotopes, or isomers thereof. Also provided herein are methods of inhibiting a component of the sterol regulatory element binding protein (SREBP) pathway, such as an SREBP or SREBP cleavage activating protein (SCAP), using these compounds, or pharmaceutically acceptable salts, solvates, tautomers, isotopes, or isomers thereof. Further provided are methods of treating a disorder in a subject in need thereof, such as liver disease, non-alcoholic steatohepatitis, insulin resistance, or cancer.

### CROSS-REFERENCE TO RELATED APPLICATIONS

This application claims the benefit of U.S. Provisional Application No. 62/935,025, filed Nov. 13, 2019; and U.S. Provisional Application No. 63/056,405, filed Jul. 24, 2020, the disclosures of which are hereby incorporated by reference in their entireties.

### FIELD OF THE INVENTION

The present disclosure relates to compounds comprising a three-ring core, their use for inhibiting components of the sterol regulatory element binding protein (SREBP) pathway, such as SREBP or SREBP cleavage activating protein (SCAP), and their use in therapeutic methods of treating conditions and disorders.

### BACKGROUND

SREBPs are membrane-bound transcription factors that regulate cholesterol, fatty acid, and triglyceride biosynthesis, and lipid uptake. Fatty acids and lipids are a source of energy and important components of many biological structures, such as lipid membranes of cells. Cholesterol is an important component of biological processes and structures. In mammals, there are three known SREBP isoforms: SREBP-1a, SREBP-1c, and SREBP-2. SREBP-1a controls a broad range of target genes that are involved in the production of fatty acids, triglycerides, phospholipids, and cholesterol. SREBP-1c primarily activates genes which control fatty acid and triglyceride synthesis. SREBP-2 activates genes involved in the synthesis of regulators of cholesterol metabolism, which has been demonstrated in mouse, human, and Drosophila studies. The activity of SREBPs is regulated by SREBP cleavage activating protein (SCAP), which transports SREBP(s) from the endoplasmic reticulum to the Golgi apparatus where the SREBP(s) are proteolytically cleaved, releasing the transcription factor domain.

The pathways regulated by SREBPs and SCAP have been implicated in disorders of metabolism, such as hypertension, dyslipidemia, obesity, type 2 diabetes, insulin resistance, fatty liver, and nonalcoholic steatohepatitis (NASH). NASH, for example, is liver inflammation and hepatocyte ballooning as a result of fat building up in the liver, which can lead to liver damage, such as cirrhosis. NASH can also be associated with other metabolism disorders, such as insulin resistance and metabolic syndrome.

The metabolism of fatty acids, cholesterol, and triglycerides may also be linked to hyperproliferative disorders, such as cancer. One characteristic of the oncogenic transformation of cancer cells is the shift of metabolism from catabolic to anabolic processes. Many cancers require synthesis of fatty acids and other lipids (such as cholesterol), and steroids (such as androgens). Thus, components of the SREBP pathway may play a role in hyperproliferative disorders, such as prostate cancer. SREBP-1c is the major transcriptional regulator of the biosynthesis of fatty acids, and expression of this transcription factor can be stimulated by androgens and epidermal growth factor in prostate cancer cells. Overexpression of SREBP-1c may drive tumorigenicity and invasion of prostate cancer cells. In addition to regulating androgen synthesis, SREBP-2 itself is also regulated by androgens in a direct feedback circuit of androgen production. However, prostate cancer cells have dysfunctional cholesterol homeostasis, resulting in accumulation of cholesterol and increased proliferation. This increase in cholesterol levels has been shown to be driven by regulated by increased SREBP-2 activity. SREBP-2 expression increases during disease progression, and is significantly higher after castration compared to prior.

Regulating components of the SREBP pathway, such as SCAP or SREBPs, is an important therapeutic approach for treating disorders, such as metabolic diseases and cancer. Thus, there is a need for compounds that can inhibit components of the SREBP pathway, such as SREBPs and SCAP.

### BRIEF SUMMARY

In some embodiments, the compound is of Formula (II):

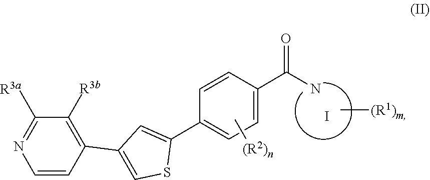

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, heterocycloalkyl, and (C₃-C₁₀)cycloalkyl, and wherein each heterocycloalkyl and cycloalkyl is unsubstituted or substituted with one or more substituents independently selected from the group consisting of alkyl, halo, and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

In some embodiments, the compound is of Formula (I):

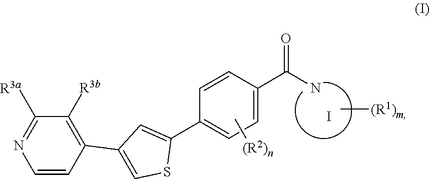

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, and (C₃-C₁₀)cycloalkyl, and wherein each heterocycloalkyl and cycloalkyl is unsubstituted or substituted with one or more substituents independently selected from the group consisting of halo and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

In some embodiments, ring I is azetidinyl, pyrrolidinyl, or piperidinyl. In certain embodiments, ring I is piperidinyl.

In further embodiments, provided herein is a pharmaceutical composition, comprising a compound as described herein, or a pharmaceutically acceptable salt, solvate, tautomer, isotope, or isomer thereof, and a pharmaceutically acceptable excipient.

In still other embodiments, provided herein is a method of inhibiting a sterol regulatory element-binding protein (SREBP), comprising contacting the SREBP or contacting an SREBP cleavage activating-protein (SCAP) with an effective amount of a compound described herein, or a pharmaceutically acceptable salt, solvate, tautomer, isotope, or isomer thereof, or a pharmaceutical composition as described herein. In some embodiments, provided herein is a compound as described herein, or a pharmaceutically acceptable salt, solvate, tautomer, isotope, or isomer thereof, for use in inhibiting a sterol regulatory element-binding protein (SREBP). In still further embodiments, provided herein is the use of a compound as described herein, or a pharmaceutically acceptable salt, solvate, tautomer, isotope, or isomer thereof, in the manufacture of a medicament for inhibiting a sterol regulatory element-binding protein (SREBP).

In certain embodiments, provided herein is a method of inhibiting the proteolytic activation of a sterol regulatory element-binding protein (SREBP), comprising contacting an SREBP cleavage activating-protein (SCAP) with an effective amount of a compound described herein, or a pharmaceutically acceptable salt, solvate, tautomer, isotope, or isomer thereof, or the pharmaceutical composition described herein. In some embodiments, provided herein is a compound as described herein, or a pharmaceutically acceptable salt, solvate, tautomer, isotope, or isomer thereof, for use in inhibiting the proteolytic activation of a sterol regulatory element-binding protein (SREBP). In some embodiments, provided herein is the use of a compound as described herein, or a pharmaceutically acceptable salt, solvate, tautomer, isotope, or isomer thereof, in the manufacture of a medicament for inhibiting the proteolytic activation of a sterol regulatory element-binding protein (SREBP).

In still further embodiments, provided herein is a method of treating a disorder in a subject in need thereof, comprising administering to the subject in need thereof an effective amount of a compound as described herein, or a pharmaceutically acceptable salt, solvate, tautomer, isotope, or isomer thereof, or a pharmaceutical composition described herein. In some embodiments, provided herein is a compound as described herein, or a pharmaceutically acceptable salt, solvate, tautomer, isotope, or isomer thereof, for use in treating a disorder in a subject in need thereof. In other embodiments, provided herein is the use of a compound as described herein, or a pharmaceutically acceptable salt, solvate, tautomer, isotope, or isomer thereof, in the manufacture of a medicament for treating a disorder in a subject in need thereof.

In yet other embodiments, provided herein is a method of treating a disorder in a subject in need thereof, wherein the disorder is mediated by a sterol regulatory element-binding protein (SREBP), comprising administering to the subject in need thereof an effective amount of a compound as described herein, or a pharmaceutically acceptable salt, solvate, tautomer, isotope, or isomer thereof, or a pharmaceutical composition as described herein. In some embodiments, provided herein is a compound as described herein, or a pharmaceutically acceptable salt, solvate, tautomer, isotope, or isomer thereof, for use in treating a disorder in a subject in need thereof. In other embodiments, provided herein is the use of a compound as described herein, or a pharmaceutically acceptable salt, solvate, tautomer, isotope, or isomer thereof, in the manufacture of a medicament for treating a disorder in a subject in need thereof.

In some embodiments of the methods, compounds for use, or uses provided herein, the disorder is Metabolic Syndrome, type 2 diabetes, obesity, liver disease, insulin resistance, adiposopathy, or dyslipidemia. In some embodiments, the liver disease is nonalcoholic steatohepatitis, liver fibrosis, or liver inflammation, or a combination thereof. In other embodiments, the disorder is a hyperproliferative disorder, such as cancer, for example, breast cancer, liver cancer, ovarian cancer, pancreatic cancer, or prostate cancer.

### DETAILED DESCRIPTION

The following description sets forth numerous exemplary configurations, methods, parameters, and the like. It should be recognized, however, that such description is not intended as a limitation on the scope of the present disclosure, but is instead provided as a description of exemplary embodiments.

### I. Compounds of Formula (I) and Formula (II)

In some embodiments, provided herein is a compound of Formula (II):

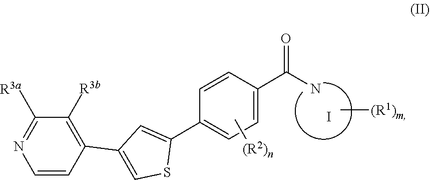

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl; R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, heterocycloalkyl, and (C₃-C₁₀)cycloalkyl, and wherein each heterocycloalkyl and cycloalkyl is unsubstituted or substituted with one or more substituents independently selected from the group consisting of alkyl, halo, and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

In some embodiments, provided herein is a compound of Formula (I):

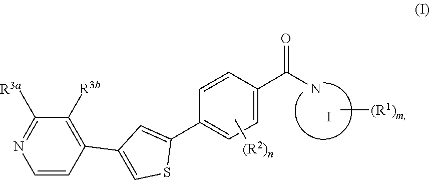

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, and (C₃-C₁₀)cycloalkyl, and wherein each heterocycloalkyl and cycloalkyl is unsubstituted or substituted with one or more substituents independently selected from the group consisting of halo and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

“Alkyl”, as used herein, refers to an unbranched or branched saturated hydrocarbon chain. Alkyl can be used alone, or as part of another radical, such as cycloalkyl-alkyl. In some embodiments, alkyl as used herein has 1 to 50 carbon atoms ((C₁₋₅₀)alkyl), 1 to 20 carbon atoms ((C₁₋₂₀)alkyl), 1 to 12 carbon atoms ((C₁₋₁₂)alkyl), 1 to 10 carbon atoms ((C₁₋₁₀)alkyl), 1 to 8 carbon atoms ((C₁₋₈)alkyl), 1 to 6 carbon atoms ((C₁₋₆)alkyl), or 1 to 4 carbon atoms ((C₁₋₄)alkyl). Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methyl pentyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

“Cycloalkyl”, as used herein, refers to a monocyclic or polycyclic saturated hydrocarbon. In some embodiments, cycloalkyl has 3 to 50 carbon atoms ((C₃₋₅₀)cycloalkyl), 3 to 20 carbon atoms ((C₃₋₂₀)cycloalkyl), 3 to 12 carbon atoms ((C₃₋₁₂)cycloalkyl), 3 to 10 carbon atoms ((C₃₋₁₀)cycloalkyl), 3 to 8 carbon atoms ((C₃₋₈)cycloalkyl), 3 to 6 carbon atoms ((C₃₋₆)cycloalkyl), or 3 to 5 carbon atoms ((C₃₋₄)cycloalkyl). Cycloalkyl includes monocyclic and polycyclic groups, such as fused bicycles, bridged rings, and spirocycles. Examples of cycloalkyl groups include cyclopropyl, cyclobutyl, cyclopentyl, cyclohexyl, octahydropentalenyl, octahydro-1H-indene, decahydronaphthalene, cubane, bicyclo[3.1.0]hexane, and bicyclo[1.1.1]pentane.

“Heterocycloalkyl”, as used herein, refers to a saturated monocyclic or polycyclic ring containing carbon and at least one heteroatom selected from the group consisting of O, N, and S. The heterocycloalkyl group may comprise 3, 4, 5, 6, 7, 8, 9, 10, 11, 12, or more ring atoms (e.g., be a 3-membered, 4-membered, 5-membered, 6-membered, 7-membered, 8-membered, 9-membered, 10-membered, 11-membered, or 12-membered heterocycloalkyl). Heterocycloalkyl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. Each ring S atom, where present, may independently be unoxidized sulfur (e.g., —S—) or a sulfur oxide, such as —S(O)—, or —S(O)₂—. In certain examples, a heterocycloalkyl has 2 to 8 ring carbon atoms and with 1 to 3 ring heteroatoms independently selected from N, O, and S. In some embodiments, heterocycloalkyl is connected through an annular carbon atom, wherein the point of attachment of the heterocycloalkyl to another group is a ring carbon atom of the heterocycloalkyl. Heterocycloalkyl includes polycyclic systems, such as bridged, fused, and spirocycles comprising at least one heteroatom in at least one of the rings. Examples of heterocycloalkyl include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxinyl, piperidinyl, morpholinyl, thiomorpholinyl, thiomorpholinyl S-oxide, thiomorpholinyl S-dioxide, piperazinyl, azepinyl, oxepinyl, diazepinyl, and tropanyl.

“Heterocycloalkenyl”, as used herein, refers to a non-aromatic monocyclic or polycyclic ring containing carbon, at least one heteroatom selected from the group consisting of O, N, and S, and at least one double bond. Each ring S atom, where present, may independently be a sulfur oxide, such as —S(O)—, or —S(O)₂—. The heterocycloalkenyl group may comprise 3, 4, 5, 6, 7, 8, 9, 10, 11, 12, or more ring atoms (e.g., be a 3-membered, 4-membered, 5-membered, 6-membered, 7-membered, 8-membered, 9-membered, 10-membered, 11-membered, or 12-membered heterocycloalkenyl). Heterocycloalkenyl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In certain examples, a heterocycloalkenyl has 2 to 8 ring carbon atoms and with 1 to 3 ring heteroatoms independently selected from N, O, and S. In some embodiments, heterocycloalkenyl is connected through an annular carbon atom, wherein the point of attachment of the heterocycloalkenyl to another group is a ring carbon atom of the heterocycloalkenyl. Heterocycloalkenyl may have one, two, three, four, five, or more double bonds, as valency permits, and each double bond independently may be between two ring carbon atoms, two ring heteroatoms, or one ring carbon atom and one ring heteroatom, as valency permits.

“Heterocyclyl” refers to a saturated or unsaturated monocyclic or polycyclic ring containing carbon and at least one heteroatom selected from the group consisting of O, N, and S. Each ring S atom, where present, may independently be a sulfur oxide, such as —S(O)—, or —S(O)₂—. Heterocyclyl includes heterocycloalkyl, heteroaryl, and non-aromatic unsaturated heterocyclic groups such as heterocycloalkenyl. The heterocyclyl group may comprise 3, 4, 5, 6, 7, 8, 9, 10, 11, 12, or more ring atoms (e.g., be a 3-membered, 4-membered, 5-membered, 6-membered, 7-membered, 8-membered, 9-membered, 10-membered, 11-membered, or 12-membered heterocyclyl), and may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In some embodiments, heterocyclyl is connected through an annular carbon atom, wherein the point of attachment of the heterocyclyl to another group is a ring carbon atom of the heterocyclyl.

“Heteroaryl”, as used herein, refers to a monocyclic or polycyclic radical comprising at least one aromatic ring, wherein the aromatic ring comprises at least one ring heteroatom independently selected from the group consisting of N, O, and S, (e.g., pyridine, pyrazine, furan, thiophene, quinoline). Each ring S atom, where present, may independently be unoxidized sulfur (e.g., —S—) or a sulfur oxide, such as —S(O)—, or —S(O)₂—. Heteroaryl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In certain examples, a heteroaryl has 3 to 8 ring carbon atoms, with 1 to 3 ring heteroatoms independently selected from N, O, and S. Heteroaryl may comprise 5, 6, 7, 8, 9, 10, 11, 12, or more annular atoms (e.g., be a 3-membered, 4-membered, 5-membered, 6-membered, 7-membered, 8-membered, 9-membered, 10-membered, 11-membered, or 12-membered heteroaryl), wherein the annular atoms are present in one or more rings. Heteroaryl may comprise, for example, 1 to 14 annular carbon atoms ((C₁₋₁₄)heteroaryl), 1 to 10 annular carbon atoms ((C₁₋₁₀)heteroaryl), 1 to 6 annular carbon atoms ((C₁₋₆)heteroaryl), 1 to 5 annular carbon atoms ((C₁₋₅)heteroaryl), or 2 to 5 annular carbon atoms ((C₂₋₅)heteroaryl). In some embodiments, heteroaryl is connected through an annular carbon atom, wherein the point of attachment of the heteroaryl to another group is a ring carbon atom of the heteroaryl. Examples of heteroaryl groups include pyridyl, pyridazinyl, pyrimidinyl, benzothiazolyl, furanyl, and pyrazolyl.

It should be understood that when a range of values is listed, it is intended to encompass each value and sub-range within the range. For example, “(C₁₋₆)alkyl” (which may also be referred to as C₁-C₆ alkyl, C₁-C₆ alkyl, or C₁₋₆ alkyl) is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Hydroxy”, as used herein, refers to the radical —OH.

“Halo”, as used herein, refers to fluoro, chloro, bromo, or iodo radicals.

“Oxo”, as used herein, refers to the radical ═O.

“Optional” or “optionally” means that the subsequently described event or circumstance may or may not occur, and that the description includes instances where the event or circumstance occurs and instances in which it does not.

In some embodiments of the compound of Formula (I) or Formula (II), the compound is a solvate. In some embodiments of the compound of Formula (I) or Formula (II), the solvate is a hydrate.

In some embodiments, provided is a pharmaceutically acceptable salt of a compound of Formula (I) or Formula (II).

Further provided herein is a pharmaceutical composition comprising a compound of Formula (I) or Formula (II), or a pharmaceutically acceptable salt, solvate, tautomer, isotope, or isomer thereof, and a pharmaceutically acceptable excipient. A pharmaceutically acceptable excipient may include, for example, an adjuvant, carrier, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans. Pharmaceutically acceptable excipients may include, but are not limited to, water, NaCl, normal saline solutions, lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates (such as lactose, amylose or starch), fatty acid esters, hydroxymethylcellulose, polyvinyl pyrrolidine, and colors.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salt” includes a salt which is generally safe, non-toxic and not biologically or otherwise undesirable, and includes that which is acceptable for veterinary use as well as human pharmaceutical use. Such salts may include acid addition salts and base addition salts. Acid addition salts may be formed with inorganic acid such as, but not limited to, hydrochloric acid, hydrobromic acid, sulfuric acid, nitric acid, phosphoric acid and the like; or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, or undecylenic acid. Salts derived from inorganic bases may include, but are not limited to, sodium, potassium, lithium, ammonium, calcium, magnesium, iron, zinc, copper, manganese, and aluminum salts. Salts derived from organic bases may include, but are not limited to, salts of primary, secondary, or tertiary amines; substituted amines including naturally occurring substituted amines; cyclic amines; ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, or N-ethylpiperidine.

In some embodiments, provided is an isotope of a compound of Formula (I) or Formula (II).

Unless otherwise stated, structures depicted herein, such as compounds of Formula (I) or Formula (II), or a pharmaceutically acceptable salt, solvate, tautomer, or isomer thereof, are also meant to include compounds which differ only in the presence of one or more isotopically enriched atoms. The compounds herein may contain unnatural proportions of atomic isotopes at one or more of the atoms that constitute such compounds. In some embodiments, the compound is isotopically-labeled, such as an isotopically-labeled compound of Formula (I) or Formula (II), or a pharmaceutically acceptable salt, solvate, tautomer, or isomer thereof, where a fraction of one or more atoms are replaced by an isotope of the same element. Exemplary isotopes that can be incorporated into compounds of the present disclosure include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³⁵S, ¹⁸F, ³⁶Cl. Certain isotope labeled compounds (e.g. ³H and ¹⁴C) may be useful in compound or substrate tissue distribution study. Incorporation of heavier isotopes such as deuterium (²H) may, in some embodiments, afford certain therapeutic advantages resulting from greater metabolic stability, for example, increased in vivo half-life, or reduced dosage requirements.

The compounds disclosed herein, such as compounds of Formula (I) or Formula (II), or a pharmaceutically acceptable salt, solvate, tautomer, or isotope thereof, may contain one or more asymmetric centers and thus may give rise to one or more isomers.

In some embodiments, provided is a tautomer of a compound of Formula (I) or Formula (II).

As described above, ring I is a 3- to 10-membered heterocycloalkyl. In certain embodiments, ring I is a 4-membered heterocycloalkyl. In certain embodiments, ring I is a 5-membered heterocycloalkyl. In certain embodiments, ring I is a 6-membered heterocycloalkyl.

In certain embodiments, ring I is a 3- to 10-membered heterocycloalkyl that comprises at least one nitrogen.

In certain embodiments, ring I is

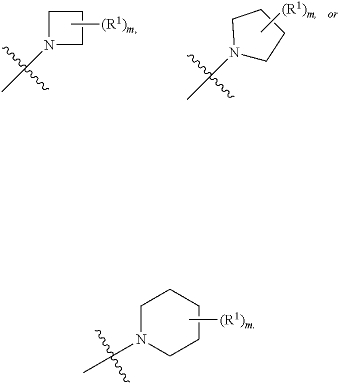

In certain embodiments, ring I is unsubstituted. In some embodiments, ring I is substituted with 1 to 8 R¹. In certain other embodiments, ring I is substituted with 0 to 1 R¹. In some embodiments, ring I is substituted with 0 to 2 R¹. In certain embodiments, ring I is substituted with 0 to 4 R¹.

As described above, each R¹ is independently halo, oxo, alkyl, or —OR⁴, wherein each alkyl is independently unsubstituted or substituted with one or more substituents independently selected from halo or —OH. In certain embodiments, —OR⁴ is —OH.

In certain embodiments, R¹ is —OH. In some embodiments, R¹ is halo. In some embodiments, R¹ is alkyl.

In certain embodiments, ring I is

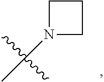 [SMILES: *N1CCC1]

In certain embodiments, ring I is

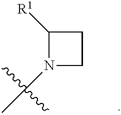

In certain embodiments, ring I is

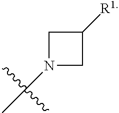

In certain embodiments, ring I is

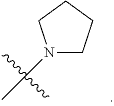 [SMILES: *N1CCCC1]

In certain embodiments, ring I is

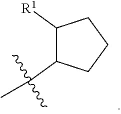

In certain embodiments, ring I is

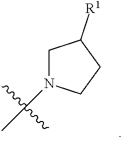

In certain embodiments, ring I is

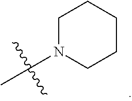 [SMILES: *N1CCCCC1]

In certain embodiments, ring I is

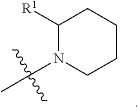

In certain embodiments, ring I is

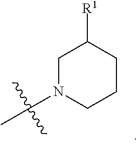

In certain embodiments, ring I is

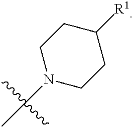

In certain embodiments, as shown herein, ring I is substituted with R^{1a} and R^{1b}, wherein R^{1a} and R^{1b} are independently halo, alkyl, or —OR⁴, wherein each alkyl is independently unsubstituted or substituted with one or more substituents independently selected from the group consisting of halo and —OH.

In certain embodiments, R^{1a} and R^{1b} are independently —OH and alkyl. In some embodiments, R^{1a} and R^{1b} are independently —OH and halo.

In certain embodiments, ring I is

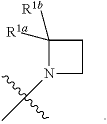

In certain embodiments, ring I is

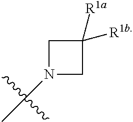

In certain embodiments, ring I is

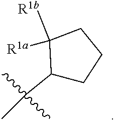

In certain embodiments, ring I is

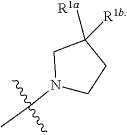

In certain embodiments, ring I is

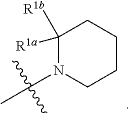

In certain embodiments, ring I is

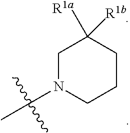

In certain embodiments, ring I is

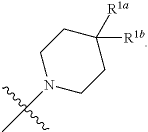

In certain embodiments, ring I is

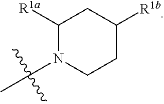

In certain embodiments, ring I is

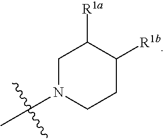

In certain embodiments, ring I is

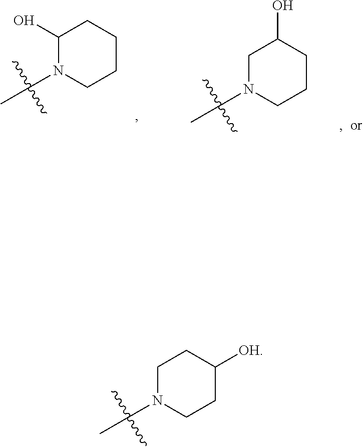 [SMILES: *N1CCCCC1O ; *N1CCCC(O)C1 ; *N1CCC(O)CC1]

In certain embodiments, ring I is

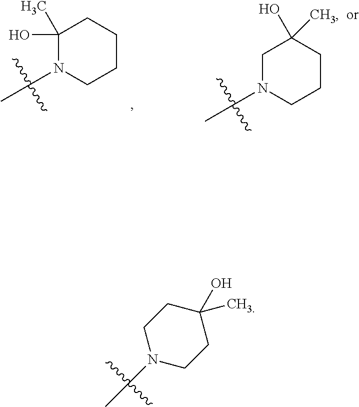

In certain embodiments, ring I is

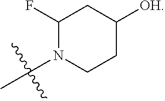 [SMILES: *N1CCC(O)CC1F]

In certain embodiments, ring I is

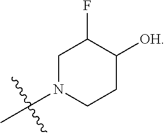 [SMILES: *N1CCC(O)C(F)C1]

In certain embodiments, ring I is

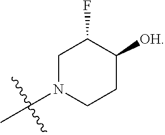 [SMILES: *N1CC[C@H](O)[C@@H](F)C1]

In some embodiments, ring I is

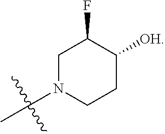 [SMILES: *N1CC[C@@H](O)[C@H](F)C1]

As described above, each R² is independently halo, alkyl, or haloalkyl. In some embodiments, R² is absent. In certain other embodiments, there is 1 R². In certain embodiments, there are 2 R².

In certain embodiments, R² is chloro.

In certain embodiments, there are 2 R², wherein both R² are halo. In certain embodiments, there are 2 R², wherein both R² are alkyl. In certain embodiments, there are 2 R², wherein both R² are haloalkyl. In certain embodiments, there are 2 R², wherein the 2 R² are a combination of halo and alkyl, halo and haloalkyl, or alkyl and haloalkyl.

In certain embodiments, R^{3a} is —CH₂—(C₃-C₁₀)cycloalkyl substituted with one or more substituents independently selected from the group consisting of halo and —OH.

In certain embodiments, R^{3a} is

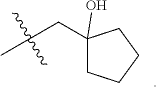 [SMILES: *CC1(O)CCCC1]

In certain embodiments, R^{3a} is (C₃-C₁₀)cycloalkyl substituted with one or more substituents independently selected from the group consisting of halo and —OR⁵. In certain embodiments, R⁵ is hydrogen. In some embodiments, R⁵ is alkyl. In still further embodiments, R⁵ is haloalkyl.

In certain embodiments, R^{3a} is a 3- to 6-membered cycloalkyl. In some embodiments, R^{3a} is a 3-membered cycloalkyl. In certain other embodiments, R^{3a} is a 4-membered cycloalkyl. In some embodiments, R^{3a} is a 5-membered cycloalkyl. In certain embodiments, R^{3a} is a 6-membered cycloalkyl.

In certain embodiments, R^{3a} is

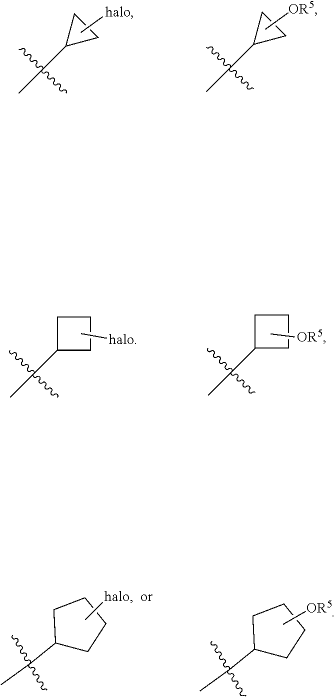

In certain embodiments, R^{3a} is a (C₃-C₁₀)cycloalkyl substituted with fluorine.

In certain embodiments, R^{3a} is a (C₃-C₁₀)cycloalkyl substituted with —OH.

In certain embodiments, R^{3a} is

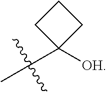 [SMILES: *C1(O)CCC1]

In certain embodiments, R^{3a} is

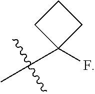 [SMILES: *C1(F)CCC1]

In certain embodiments, R^{3a} is (C₃-C₁₀)cycloalkyl substituted with one or more alkyl, wherein the cycloalkyl and each alkyl are independently unsubstituted or substituted with one or more substituents independently selected from the group consisting of halo and —OR⁶. In certain embodiments, R⁶ is hydrogen. In some embodiments, R⁶ is alkyl. In still further embodiments, R⁶ is haloalkyl.

In certain embodiments, R^{3a} is (C₃-C₁₀)cycloalkyl substituted with one or more methyl, wherein each cycloalkyl and methyl are independently unsubstituted or substituted with one or more substituents independently selected from the group consisting of halo and —OR⁶. In some embodiments, —OR⁶ is —OH.

In certain embodiments, R^{3a} is

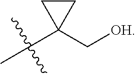 [SMILES: *C1(CO)CC1]

In certain embodiments, R^{3a} is

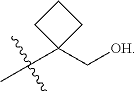 [SMILES: *C1(CO)CCC1]

In certain embodiments, R^{3a} is heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH. In certain embodiments, R⁷ is hydrogen. In some embodiments, R⁷ is alkyl. In still further embodiments, R⁷ is haloalkyl.

In certain embodiments, R^{3a} is a 4- to 6-membered heterocycloalkyl. In some embodiments, R^{3a} is a 4-membered heterocycloalkyl. In certain other embodiments, R^{3a} is a 5-membered heterocycloalkyl. In some embodiments, R^{3a} is a 6-membered heterocycloalkyl.

In certain embodiments, R^{3a} is heterocycloalkyl, wherein each heteroatom is oxygen.

In certain embodiments, R^{3a} is an unsubstituted heterocycloalkyl.

In certain embodiments, R^{3a} is

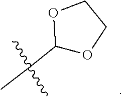 [SMILES: *C1OCCO1]

In certain embodiments, R^{3a} is

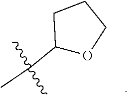 [SMILES: *C1CCCO1]

In certain embodiments, R^{3a} is

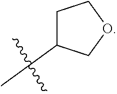 [SMILES: *C1CCOC1]

In certain embodiments, R^{3a} is

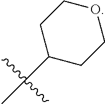 [SMILES: *C1CCOCC1]

In certain embodiments, R^{3a} is a heterocycloalkyl substituted with one —OH.

In certain embodiments, R^{3a} is

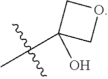 [SMILES: *C1(O)COC1]

In certain embodiments, R^{3a} is H

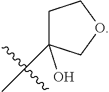 [SMILES: *C1(O)CCOC1]

In certain embodiments, R^{3a} is

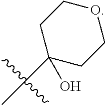 [SMILES: *C1(O)CCOCC1]

In certain embodiments, R^{3a} is OR⁹, wherein R⁹ is heterocycloalkyl, (C₃₋₁₀)cycloalkyl, or alkyl, and the alkyl is unsubstituted or substituted with one or more Rio independently selected from hydrogen, —OH, halo, or (C₃-C₁₀)cycloalkyl, wherein each heterocycloalkyl and cycloalkyl is unsubstituted or substituted with one or more substituents independently selected from the group consisting of halo and —OH;

In certain embodiments, R^{3a} is —OR⁹, wherein R⁹ is an unsubstituted heterocycloalkyl. In some embodiments, R^{3a} is —OR⁹, wherein R⁹ is an unsubstituted (C₃₋₁₀)cycloalkyl.

In certain embodiments, R^{3a} is —OR⁹, wherein R⁹ is an unsubstituted alkyl. In some embodiments, R^{3a} is —OR⁹, wherein R⁹ is an alkyl substituted with one or more substituents independently selected from the group consisting of —OH, halo, and (C₃-C₁₀)cycloalkyl, and wherein each heterocycloalkyl and cycloalkyl is unsubstituted or substituted with one or more substituents independently selected from the group consisting of halo and —OH.

In some embodiments, R^{3a} is —OR⁹, wherein R⁹ is an alkyl substituted with one or more substituents independently selected from the group consisting of —OH, halo, heterocycloalkyl, and (C₃-C₁₀)cycloalkyl, and wherein each heterocycloalkyl and cycloalkyl is unsubstituted or substituted with one or more substituents independently selected from the group consisting of alkyl, halo, and —OH.

In certain embodiments, R^{3a} is

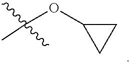 [SMILES: *OC1CC1]

In certain embodiments, R^{3a} is

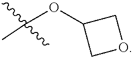 [SMILES: *OC1COC1]

In certain embodiments, R^{3a} is —OR⁹, wherein R⁹ is a heterocycloalkyl substituted with —OH.

In certain embodiments, R^{3a} is

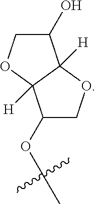 [SMILES: *OC1COC2C(O)COC12]

In certain embodiments, R^{3a} is

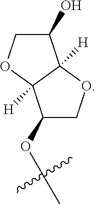 [SMILES: *O[C@@H]1CO[C@H]2[C@@H]1OC[C@H]2O]

In certain embodiments, R^{3a} is —OR⁹, wherein R⁹ is an unsubstituted (C₃₋₁₀)cycloalkyl.

In certain embodiments, R^{3a} is

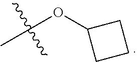 [SMILES: *OC1CCC1]

In certain embodiments, R^{3a} is —OR⁹, wherein R⁹ is a (C₃-C₁₀)cycloalkyl substituted with one —OH or one halo. In some embodiments, halo is fluoro.

In certain embodiments, R^{3a} is

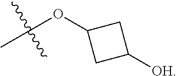 [SMILES: *OC1CC(O)C1]

In some embodiments, R^{3a} is

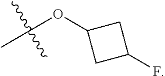 [SMILES: *OC1CC(F)C1]

In certain embodiments, R^{3a} is

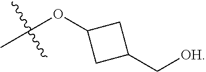 [SMILES: *OC1CC(CO)C1]

In certain embodiments, R^{3a} is

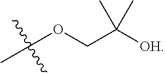 [SMILES: *OCC(C)(C)O]

In certain embodiments, R^{3a} is

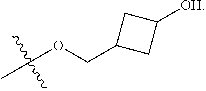 [SMILES: *OCC1CC(O)C1]

In certain embodiments, R^{3a} is —OR⁹, wherein R⁹ is alkyl. In some embodiments, R^{3a} is —OR⁹, wherein R⁹ is alkyl substituted with (C₃-C₁₀)cycloalkyl, wherein each cycloalkyl is further substituted with one or more substituents independently selected from the group consisting of alkyl, halo, and —OH. In some embodiments, R^{3a} is —OR⁹, wherein R⁹ is alkyl substituted with heterocycloalkyl, wherein each heterocycloalkyl is further substituted with one or more substituents independently selected from the group consisting of alkyl, halo, and —OH.

In certain embodiments, R^{3a} is

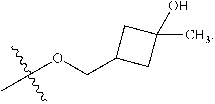 [SMILES: *OCC1CC(C)(O)C1]

In certain embodiments, R^{3a} is

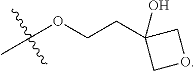 [SMILES: *OCCC1(O)COC1]

In certain embodiments, R^{3b} is hydrogen, or together with R^{3a} and the atoms to which they are attached forms a non-aromatic heterocyclyl group, wherein the non-aromatic heterocyclyl group is unsubstituted or substituted with one or more halo.

In certain embodiments, R^{3b} is hydrogen.

In certain embodiments, R^{3b} together with R^{3a} and the atoms to which they are attached forms a non-aromatic heterocyclyl group. In some embodiments, the non-aromatic heterocyclyl group is unsubstituted.

In certain embodiments, the non-aromatic heterocyclyl group formed by R^{3b} and R^{3a} together is a 6-membered non-aromatic heterocyclyl group.

In certain embodiments, the non-aromatic heterocyclyl group comprises two heteroatoms. In some embodiments, the two heteroatoms are independently O or N. In certain other embodiments, both heteroatoms are O. In some embodiments, both heteroatoms are N.

In certain embodiments, the non-aromatic heterocyclyl group formed by R^{3b} and R^{3a} together is

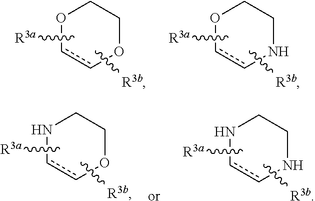

In certain embodiments, the non-aromatic heterocyclyl group formed by R^{3b} and R^{3a} together is substituted with halo.

In certain embodiments, the non-aromatic heterocyclyl group formed by R^{3b} and R^{3a} together is substituted with alkyl. In some embodiments, the alkyl is unsubstituted. In other embodiments, the alkyl is substituted with one or more halo. In some embodiments, the alkyl is substituted with one or more —OH.

In certain embodiments, the non-aromatic heterocyclyl group formed by R^{3b} and R^{3a} together is

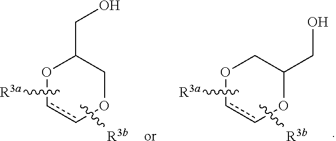

In certain embodiments, the non-aromatic heterocyclyl group formed by R^{3b} and R^{3a} together is

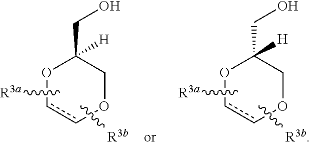

In certain embodiments, each of R⁴, R⁵, R⁶, and R⁷ are independently hydrogen, alkyl, haloalkyl, or —C(O)CHR⁸—NH₂, wherein R⁸ is a (C₁-C₆)alkyl.

In certain embodiments, each of R⁴, R⁵, R⁶, and R⁷ are independently or

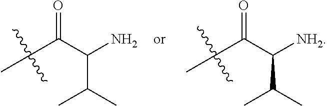

In certain embodiments, R^{3a} is

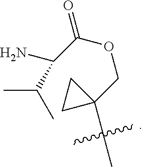 [SMILES: *C1(COC(=O)[C@@H](N)C(C)C)CC1]

In certain embodiments, —OR⁴ is

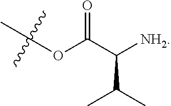 [SMILES: *OC(=O)[C@@H](N)C(C)C]

In certain embodiments, the present disclosure provides a compound, and pharmaceutically acceptable salts, solvates, tautomers, isotopes, or isomers thereof, that is

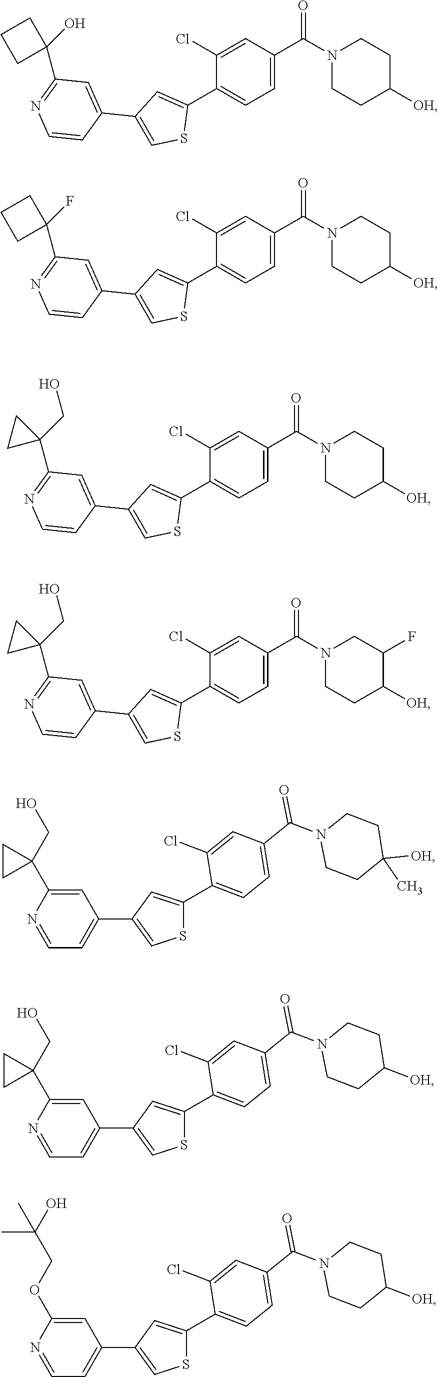

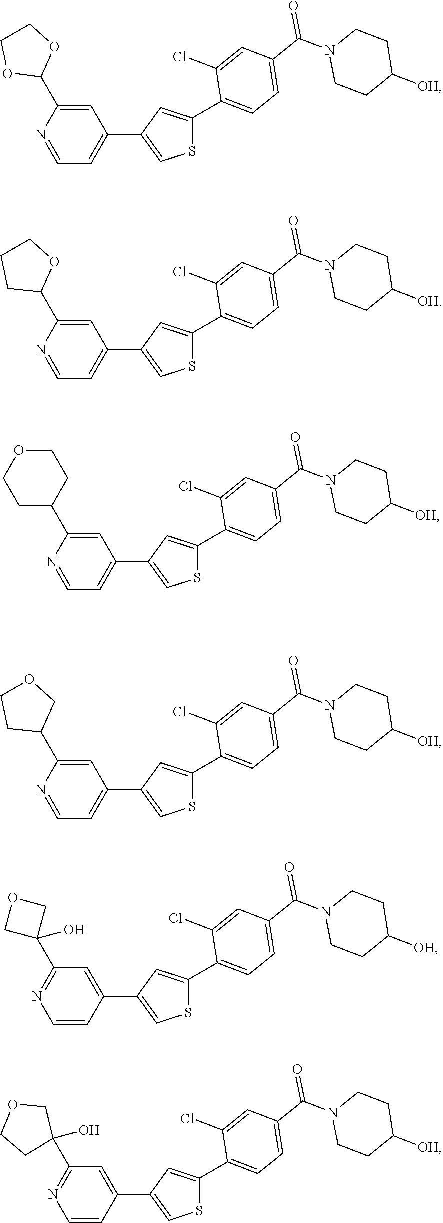 [SMILES: O=C(c1ccc(-c2cc(-c3ccnc(C4OCCO4)c3)cs2)c(Cl)c1)N1CCC(O)CC1 ; O=C(c1ccc(-c2cc(-c3ccnc(C4CCCO4)c3)cs2)c(Cl)c1)N1CCC(O)CC1 ; O=C(c1ccc(-c2cc(-c3ccnc(C4CCOCC4)c3)cs2)c(Cl)c1)N1CCC(O)CC1 ; O=C(c1ccc(-c2cc(-c3ccnc(C4CCOC4)c3)cs2)c(Cl)c1)N1CCC(O)CC1 ; O=C(c1ccc(-c2cc(-c3ccnc(C4(O)COC4)c3)cs2)c(Cl)c1)N1CCC(O)CC1 ; O=C(c1ccc(-c2cc(-c3ccnc(C4(O)CCOC4)c3)cs2)c(Cl)c1)N1CCC(O)CC1]

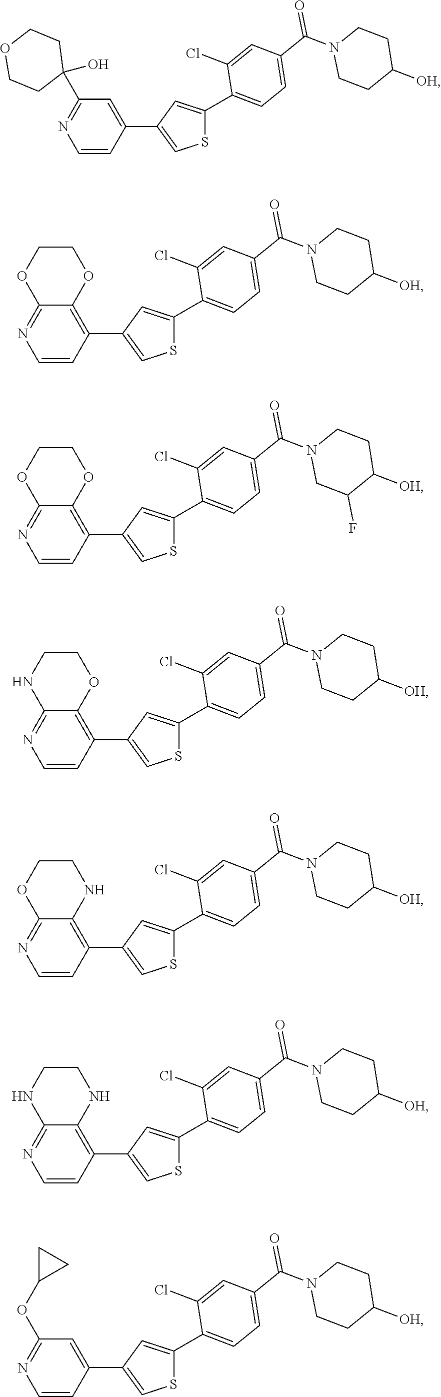

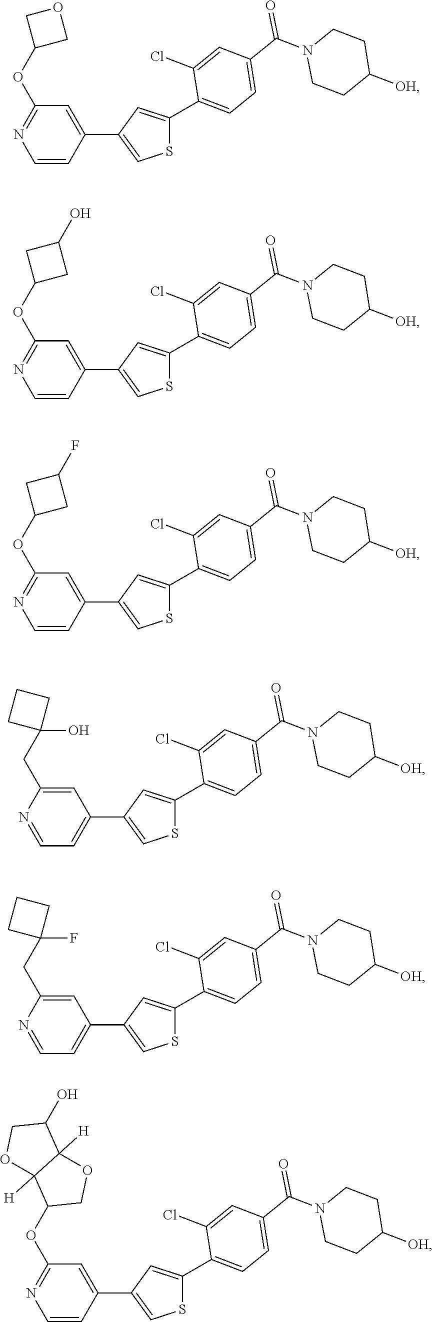

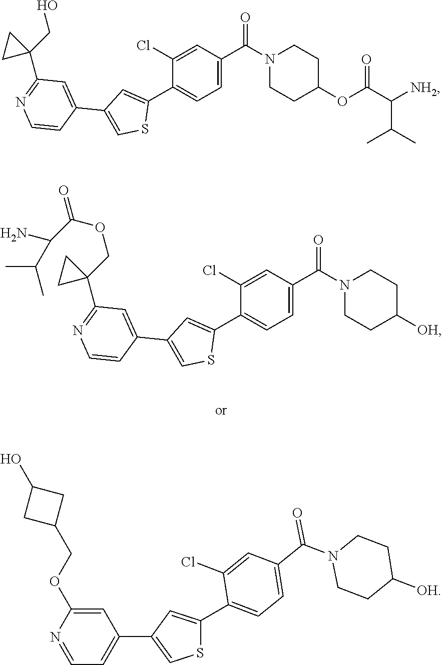

In certain embodiments, the present disclosure provides a compound, and pharmaceutically acceptable salts, solvates, tautomers, isotopes, or isomers thereof, that is

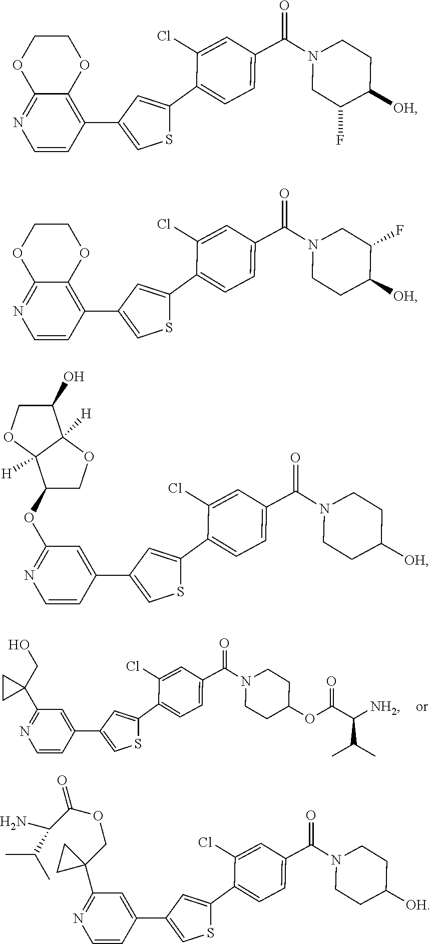

In certain embodiments, the present disclosure provides a compound, and pharmaceutically acceptable salts, solvates, tautomers, isotopes, or isomers thereof, that is

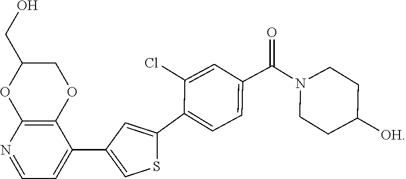 [SMILES: O=C(c1ccc(-c2cc(-c3ccnc4c3OCC(CO)O4)cs2)c(Cl)c1)N1CCC(O)CC1]

In certain embodiments, the present disclosure provides a compound, and pharmaceutically acceptable salts, solvates, tautomers, isotopes, or isomers thereof, that is

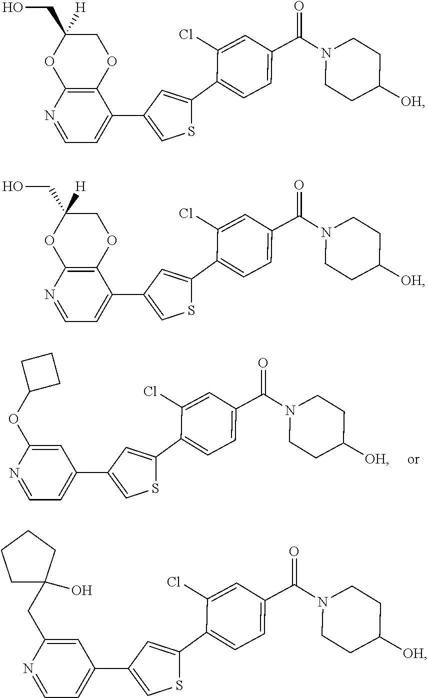

In certain embodiments, the present disclosure provides a compound, and pharmaceutically acceptable salts, solvates, tautomers, isotopes, or isomers thereof, that is

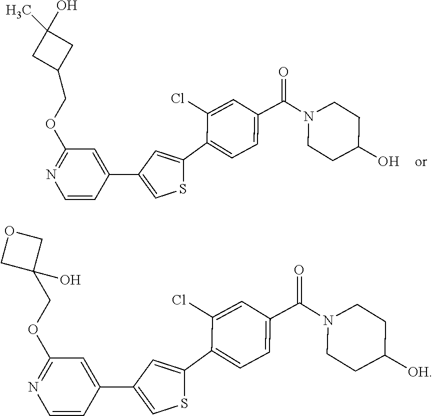 [SMILES: CC1(O)CC(COc2cc(-c3csc(-c4ccc(C(=O)N5CCC(O)CC5)cc4Cl)c3)ccn2)C1 ; O=C(c1ccc(-c2cc(-c3ccnc(OCC4(O)COC4)c3)cs2)c(Cl)c1)N1CCC(O)CC1]

Further provided are pharmaceutical compositions comprising any of the compounds disclosed herein, such as a compound of Formula (I) or Formula (II), or a pharmaceutically acceptable salt, solvate, tautomer, isotope, or isomer thereof, and a pharmaceutically acceptable excipient.

The compounds disclosed herein, such as a compound of Formula (I) or Formula (II), or a pharmaceutically acceptable salt, solvate, tautomer, isotope, or isomer thereof, may be prepared, for example, through the reaction routes depicted in General Schemes I and II.

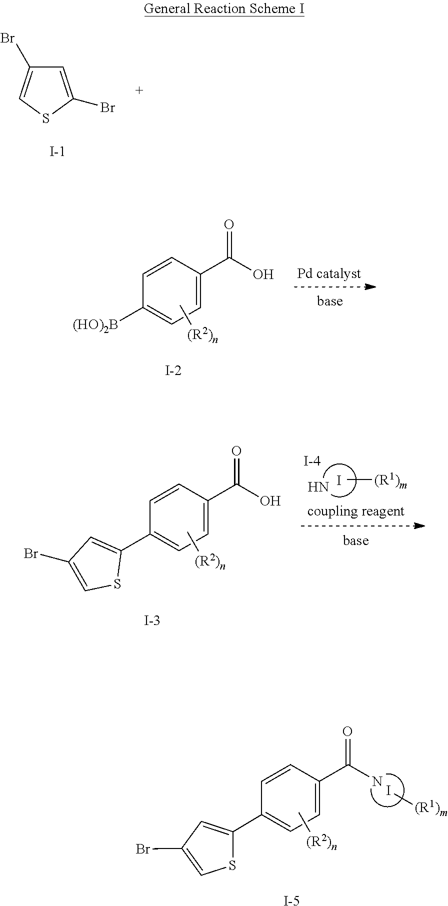

General Reaction Scheme I provides a route to compound I-5, which is an example of an intermediate used to access compounds of Formula (I) or Formula (II), or a pharmaceutically acceptable salt, solvate, tautomer, isotope, or isomer thereof as described herein. Compound I-1 is coupled with compound I-2 in the presence of a palladium catalyst and base to produce compound I-3. In the next step, compound I-3 is coupled to amine I-4 using an amide coupling reagent and base to produce compound I-5. Suitable palladium catalysts for the first step may include, for example, tetrakis(triphenylphosphine)palladium(0). Suitable bases for the first step may include, for example, aqueous sodium carbonate. Suitable amide coupling reagents for the second step may include, for example, 1-[bis(dimethylamino)methylene]-1H-1,2,3-triazolo[4,5-b]pyridinium 3-oxide hexafluorophosphate (HATU). Suitable bases for the second step may include, for example, N,N-Diisopropylethylamine (DIPEA). Any of the steps depicted in General Reaction Scheme I may further include a solvent, for example, dimethylformamide (DMF) or dioxane. In some embodiments, the reactions are carried out between room temperature and 100° C., for 16 hours. In some embodiments, R² is Cl, ring I is piperidinyl, and R¹ is —OH. In other embodiments, R² is Cl, ring I is piperidinyl, and (R¹)ₘ is one or more of the groups selected from —OH, alkyl, or halo. In some embodiments where R¹ is —OH, ring I is further modified after step two.

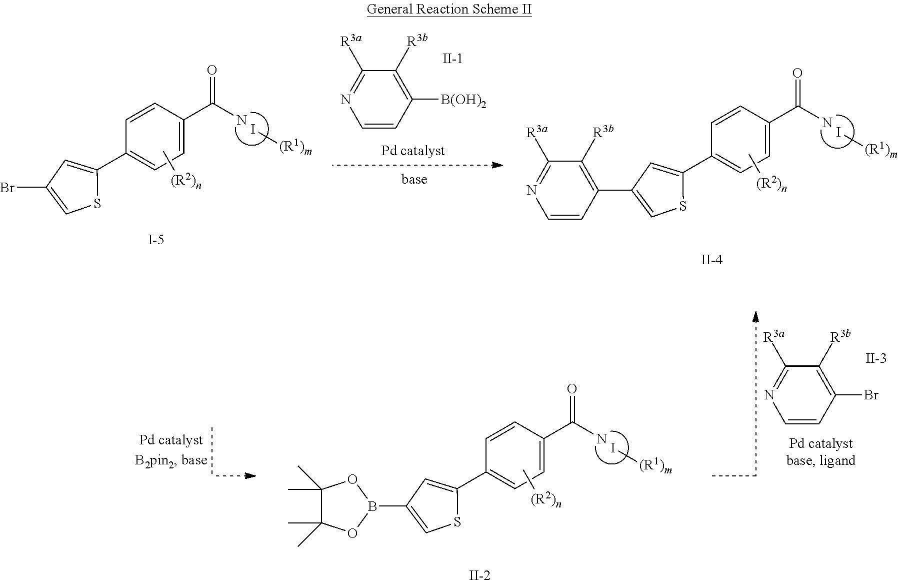

General Reaction Scheme II provides two routes to compound II-4, which is an example of a compound of Formula (I) or Formula (II), or a pharmaceutically acceptable salt, solvate, tautomer, isotope, or isomer thereof as described herein. Compound I-5 may be prepared, for example, as described in General Reaction Scheme I above. Compound II-4 may be prepared, for example, following the top route, by reacting compound I-4 with boronic acid II-1 in the presence of a palladium catalyst and a base. Suitable palladium catalysts for the top route may include, for example, tetrakis(triphenylphosphine)palladium(0). Suitable bases for the top route may include, for example, aqueous potassium carbonate. In the second route (bottom), compound I-5 is converted to its corresponding boronic ester (compound II-2) in the presence of a palladium catalyst, a base, and Bis(pinacolato)diboron (B₂pin₂). In some embodiments, II-2 may be the corresponding boronic acid. In the second step of the bottom route, II-2 is coupled to bromopyridine II-3 in the presence of a palladium catalyst, a base, and a ligand. Suitable palladium catalysts for the bottom route may include, for example, tetrakis(triphenylphosphine)palladium(0) or [1,1′-Bis(diphenylphosphino)ferrocene]palladium(II) dichloride (PdCl₂(dppf)). Suitable bases for the bottom route may include, for example, potassium acetate or aqueous potassium carbonate. Suitable ligands for the bottom route may include, for example, Xantphos. Any of the steps depicted in General Reaction Scheme II may further include a solvent, for example, tetrahydrofuran (THF) or dioxane. In some embodiments, the reactions are carried out between 80° C. and 100° C., for 16 hours. In some embodiments, the substituents R^{3a}, R^{3b}, and R¹ of compound II-4 may be independently modified after completion of the top or bottom routes.

### II. Methods of Using Compounds of Formula (I) or Formula (II) and Pharmaceutical Compositions Comprising Compounds of Formula (I) or Formula (II)

Provided herein are methods of using the compounds disclosed herein, such as compounds of Formula (I) or Formula (II), or a pharmaceutically acceptable salt, solvate, tautomer, isotope, or isomer thereof, or a pharmaceutical composition comprising any of the foregoing and a pharmaceutically acceptable excipient. These include methods of inhibiting a component of the SREBP pathway, such as an SREBP or SCAP; and methods of treating a disorder in a subject in need thereof. In some embodiments, the disorder is mediated by an SREBP or SCAP.

The terms “treat,” “treating,” or “treatment” refers to any indicia of success in the amelioration of a disorder (such as injury, disease pathology, or condition), including any objective or subjective parameter such as abatement; remission; diminishing of symptoms or making the disorder more tolerable to the subject; slowing or stopping the rate of degeneration, decline, or development; slowing the progression of disorder; making the final point of degeneration less debilitating; improving a subject's physical or mental well-being; or relieving or causing regression of the disorder. The treatment of symptoms, including the amelioration of symptoms, can be based on objective or subjective parameters, which may include the results of a physical examination, a neuropsychiatric exam, and/or a psychiatric evaluation. Certain methods and uses disclosed herein may treat cancer by, for example, decreasing the incidence of cancer, causing remission of cancer, slowing the rate of growth of cancer cells, slowing the rate of spread of cancer cells, reducing metastasis, or reducing the growth of metastatic tumors, reducing the size of one or more tumors, reducing the number of one or more tumors, or any combinations thereof.

The embodiments described herein for methods of treatment should also be considered to apply to the use of compounds of Formula (I) or Formula (II), or a pharmaceutically acceptable salt, solvate, tautomer, isotope, or isomer thereof, or a pharmaceutical composition comprising any of the forgoing, for the treatment of disorders; and the use of compounds of Formula (I), or a pharmaceutically acceptable salt, solvate, tautomer, isotope, or isomer thereof, or a pharmaceutical composition comprising any of the forgoing, for inhibiting an SREBP or inhibiting the proteolytic activation of an SREBP; and other uses of compounds of Formula (I) or Formula (II), or a pharmaceutically acceptable salt, solvate, tautomer, isotope, or isomer thereof, or a pharmaceutical composition comprising any of the forgoing, as described herein; and the use of compounds of Formula (I) or Formula (II), or a pharmaceutically acceptable salt, solvate, tautomer, isotope, or isomer thereof, in the manufacture of medicaments.

A. Inhibiting SREBP or SCAP

Provided herein are uses and methods of inhibiting a component of the SREBP pathway, such as an SREBP or SCAP. In some embodiments, a combination of an SREBP and SCAP is inhibited. Such methods may include contacting an SREBP with a compound of Formula (I) or Formula (II), or a pharmaceutically acceptable salt, solvate, tautomer, isotope, or isomer thereof, or a pharmaceutical composition comprising any of the forgoing and a pharmaceutically acceptable excipient. Such uses and methods may also include contacting SCAP with a compound of Formula (I) or Formula (II), or a pharmaceutically acceptable salt, solvate, tautomer, isotope, or isomer thereof, or a pharmaceutical composition comprising any of the forgoing and a pharmaceutically acceptable excipient.

In certain embodiments, a compound of Formula (I) or Formula (II), or a pharmaceutically acceptable salt, solvate, tautomer, isotope, or isomer thereof is administered to a subject in need thereof to inhibit a component of the SREBP pathway. In other embodiments, a pharmaceutical composition comprising a pharmaceutically acceptable excipient and a compound of Formula (I) or Formula (II), or a pharmaceutically acceptable salt, solvate, tautomer, isotope, or isomer thereof, is administered to the subject in need thereof. In certain embodiments, the amount of the compound or pharmaceutically acceptable salt, solvate, tautomer, isotope, or isomer thereof, relative to the subject's body mass, is between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof is administered to a subject in need thereof to inhibit a component of the SREBP pathway. In certain embodiments, the compound or pharmaceutically acceptable salt, solvate, tautomer, isotope, or isomer thereof, is administered as a pharmaceutical composition, as described herein.

The component of the SREBP pathway that is inhibited by the methods and uses described herein may be an SREBP or SCAP. In some embodiments, an SREBP is inhibited. The SREBP may be, for example, an SREBP-1 (such as SREBP-1a or SREBP-1c) or SREBP-2. In certain variations, two or three of SREBP-1a, SREBP-1c, and SREBP-2 are inhibited. In some embodiments, the component is an SREBP-1. In other embodiments, the SREBP is SREBP-1a. In certain embodiments, the component is SREBP-1c. In still other embodiments, the SREBP is SREBP-2. In other embodiments, the component of the SREBP pathway is SCAP. In some embodiments, both an SREBP and SCAP are inhibited. In certain embodiments, two or three of SREBP-1a, SREBP-1c, and SREBP-2 are inhibited, and SCAP is inhibited.

Inhibition of a component of the SREBP pathway, such as an SREBP or SCAP, may include partial inhibition or full inhibition. Partial inhibition may include reducing the activity of a component of the SREBP pathway to a level that is still detectable. Full inhibition may include stopping all activity of a component of the SREBP pathway (such as stopping the activity of an SREBP or SCAP), or reducing the activity of a component of the SREBP pathway to a level below detection. Inhibition of a component of the SREBP pathway may be measured directly or indirectly, using any methods known in the art.

In some embodiments, inhibition of a component of the SREBP pathway is measured directly, for example by measuring the product of a reaction catalyzed by an SREBP pathway component. Inhibition of SREBP activation (for example, by inhibiting SCAP) may in some embodiments be demonstrated by western blotting and quantitatively assessing the levels of full-length and cleaved SREBP-1 and/or SREBP-2 proteins from a cell line (such as a hepatic cell lines) or primary cells (such as primary hepatocytes of mouse, rat or human origin).

In some embodiments, inhibition of a component of the SREBP pathway is measured indirectly, for example by measuring the level of expression of one or more genes that are regulated by SREBP. The inhibition of a component of the SREBP pathway, such as an SREBP or SCAP, may reduce the expression of one or more genes that are regulated by an SREBP, for example an SREBP-1 (such as SREBP-1a or SREBP-1c) or SREBP-2. SCAP plays a role in activating SREBPs, thus inhibiting the activity of SCAP may reduce the expression of one or more genes that are regulated by an SREBP. SREBP pathway inhibition may also be determined by assessing gene transcription levels of one or more target genes of SREBP-1 and/or SREBP-2, such as one or more of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, or ACACB. The transcription levels may be assessed, for example, by transcriptomic analysis, including but not limited to q-PCR. A reduction in one, two, three, four, five, or more of these genes may indicate inhibition of SREBP activation. This evaluation of endogenous SREBP gene expression may be assessed in cell lines (such as hepatic cell lines) or primary cells (such as primary hepatocytes of mouse, rat, or human origin). In some embodiments, the gene transcription levels of PCSK9 or PNPLA3, or a combination thereof, are evaluated.

Therefore, provided herein are uses and methods of reducing the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB, comprising contacting an SREBP or SCAP with a compound of Formula (I) or Formula (II), or a pharmaceutically acceptable salt, solvate, tautomer, isotope, or isomer thereof. In some embodiments, the expression of PCSK9 is reduced. In other embodiments, the expression of PNPLA3 is reduced. In still further embodiments, both the expression of PCSK9 and PNPLA3 are reduced. In certain embodiments, one or more SREBP is contacted, for example an SREBP-1 (such as SREBP-1a or SREBP-1c) or SREBP-2, or any combinations thereof. In other embodiments, SCAP is contacted. In still further embodiments, one or more of SREBP-1a, SREBP-1c, SREBP-2, and SCAP is contacted. In certain embodiments, inhibition of a component of the SREBP pathway may treat a disorder mediated by an SREBP, such as the disorders as described herein. Thus, in certain embodiments, expression of one or more genes as described above is reduced in a subject in need thereof.

Another method of indirectly detecting SREBP pathway inhibition may include: Serum-starving a hepatic cell line (HepG2) expressing luciferase under the control of the LSS-promoter to induce SREBP activation and increased luciferase expression. The cells may then be treated with a compound, such as a compound of Formula (I) or Formula (II), or a pharmaceutically acceptable salt, solvate, tautomer, isotope, or isomer thereof. Following treatment, a reduction of luciferase activity reflects inhibition of SREBP activation, and non-cytotoxicity of the compound can be assessed by LDH release.

B. Treating a Disorder

In other embodiments, provided herein are uses and methods of treating a disorder in a subject in need thereof, comprising administering to the subject in need thereof a compound of Formula (I) or Formula (II), or a pharmaceutically acceptable salt, solvate, tautomer, isotope, or isomer thereof. In certain embodiments, provided herein are uses and methods of treating a disorder in a subject in need thereof, comprising administering to the subject in need thereof a pharmaceutical composition comprising a compound of Formula (I) or Formula (II), or a pharmaceutically acceptable salt, solvate, tautomer, isotope, or isomer thereof, and a pharmaceutically acceptable excipient. In some embodiments, the disorder is mediated by an SREBP.

The uses and methods of treatment describe herein may use a compound of Formula (I) or Formula (II), or a pharmaceutically acceptable salt, solvate, tautomer, isotope, or isomer thereof, or a pharmaceutical composition comprising a compound of Formula (I) or Formula (II), or a pharmaceutically acceptable salt, solvate, tautomer, isotope, or isomer thereof, and a pharmaceutically acceptable excipient.

1. Metabolic Disorders

In some embodiments, the disorder is a metabolic disorder, such as a disorder that affects lipid metabolism, cholesterol metabolism, or insulin metabolism. In certain embodiments, the disorder is related to lipid metabolism, cholesterol metabolism, or insulin metabolism, for example, liver disease as a result of the buildup of fat in the liver, or cardiovascular disease.

In some embodiments, the disorder is a liver disease, such as chronic liver disease. In some embodiments, the liver disease is mediated by a component of the SREBP pathway, such as an SREBP or SCAP. In some embodiments, the liver disease is mediated by an SREBP. In certain embodiments, the liver disease is mediated by a downstream gene target of an SREBP, such as PNPLA-3. In other embodiments, the liver disease is mediated by SCAP. Thus, in some embodiments, provided herein are uses and methods of treating a liver disease in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. The chronic liver disease may be, for example, primary alcoholic liver disease, nonalcoholic fatty liver disease (NAFLD), or nonalcoholic steatohepatitis (NASH). In some embodiments, the liver disease is liver fat, liver inflammation, or liver fibrosis, or a combination thereof.

In certain embodiments, the liver disease is non-alcoholic fatty liver disease (NAFLD). NAFLD is a group of conditions that are related to fat buildup in the liver. Non-alcoholic steatohepatitis (NASH) is a form of NAFLD which includes liver inflammation. In NASH, the liver inflammation may lead to liver damage and scarring, which can be irreversible, and it can also progress to cirrhosis and liver failure. NAFLD and NASH are associated with metabolic disorders such as obesity, dyslipidemia, insulin resistance, and type 2 diabetes. Other disorders associated with NAFLD and NASH include increased abdominal fat and high blood pressure. In some embodiments, NASH is mediated by a component of the SREBP pathway, such as an SREBP or SCAP.

In other embodiments, provided herein are uses and methods of treating NASH in a subject in need thereof, comprising administering to the subject in need thereof a compound of Formula (I) or Formula (II), or a pharmaceutically acceptable salt, solvate, tautomer, isotope, or isomer thereof; or a pharmaceutical composition comprising any of the foregoing and a pharmaceutically acceptable excipient. Treatment of NASH may include reduction in average liver fat content, which may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation as evaluated through histological scoring of liver biopsy; reduction of liver fibrosis as evaluated through histological scoring of liver biopsy; reduction of liver fat content as evaluated through histological scoring of liver biopsy; or any combinations thereof. Treatment of NASH may be evaluated using the NAFLD activity score (NAS); or steatosis, activity, and fibrosis score (SAF); or other NASH diagnostic and/or scoring metrics (such as FIB4 or ELF).

Further provided herein are uses and methods of treating a disorder in a subject in need thereof, wherein the disorder is liver fibrosis associated with NASH, comprising administering to the subject in need thereof a compound of Formula (I) or Formula (II), or a pharmaceutically acceptable salt, solvate, tautomer, isotope, or isomer thereof; or a pharmaceutical composition comprising any of the foregoing and a pharmaceutically acceptable excipient. In some embodiments, the liver fibrosis is mediated by SREBP. Treatment of liver fibrosis may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation and/or fibrosis as evaluated through histological scoring of liver biopsy; or any combinations thereof

Further provided herein are uses and methods of treating a disorder in a subject in need thereof, wherein the disorder is fatty liver disease, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. In some embodiments, the fatty liver disease is mediated by SREBP. In certain embodiments, a subject may have fatty liver disease when the fat content of the subject's liver is 5% or greater. In some embodiments, a subject with fatty liver disease has NASH, or liver fibrosis associated with NASH. In certain embodiments, a subject with fatty liver disease has not been diagnosed with NASH or liver fibrosis associated with NASH. Treatment of fatty liver disease may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation as evaluated through histological scoring of liver biopsy; reduction of liver fibrosis as evaluated through histological scoring of liver biopsy; reduction of liver fat content as evaluated through histological scoring of liver biopsy; or any combinations thereof.

In some embodiments of the uses and methods of treating liver disease provided herein, such as methods of treating liver fibrosis, fatty liver disease, or NASH, the subject is administered between about 0.01 mg/kg to about 100 mg/kg of compound of Formula (I) or Formula (II), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

Other metabolic disorders which may be treated with the compounds or pharmaceutical compositions described herein may include, for example, insulin resistance, hyperglycemia, diabetes mellitus, dyslipidemia, adiposopathy, obesity, and Metabolic Syndrome. In some embodiments, the metabolic disorder is mediated by a genetic factor. In other embodiments, the metabolic disorder is mediated by one or more environmental factors, such as a diet rich in fat, or a diet rich in sugar, or a combination thereof. In some embodiments, the metabolic disorder is mediated by SREBP. In some embodiments, the diabetes mellitus is type I diabetes. In certain embodiments, the diabetes mellitus is type II diabetes.

Provided herein are uses and methods of treating diabetes in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. Diabetes (also known as diabetes mellitus) refers to a disease or condition that is generally characterized by metabolic defects in production and utilization of glucose, which result in the failure to maintain appropriate blood sugar levels in the body. In some embodiments, the diabetes is type II diabetes, which is characterized by insulin resistance, in which insulin loses its ability to exert its biological effects across a broad range of concentrations. In some embodiments, the diabetes is mediated by a component of the SREBP pathway, such as an SREBP or SCAP.

Further provided herein are uses and methods of treating insulin resistance in a subject in need thereof, comprising administering to the subject in need thereof a compound of Formula (I) or Formula (II), or a pharmaceutically acceptable salt, solvate, tautomer, isotope, or isomer thereof; or a pharmaceutical composition comprising any of the foregoing and a pharmaceutically acceptable excipient. Insulin resistance has been hypothesized to unify the clustering of hypertension, glucose intolerance, hyperinsulinemia, increased levels of triglyceride, decreased HDL cholesterol, and central and overall obesity. “Metabolic Syndrome” refers to a similar clustering of conditions, which may include abdominal obesity, hypertension, high blood sugar, high serum triglycerides (such as elevated fasting serum triglycerides), and low HDL levels, and is associated with a risk of developing cardiovascular disease and/or type II diabetes. Further provided herein are uses and methods of treating Metabolic Syndrome in a subject in need thereof, comprising administering to the subject in need thereof a compound of Formula (I) or Formula (II), or a pharmaceutically acceptable salt, solvate, tautomer, isotope, or isomer thereof; or a pharmaceutical composition comprising any of the foregoing and a pharmaceutically acceptable excipient. In some embodiments, the Metabolic Syndrome or insulin resistance is mediated by a component of the SREBP pathway, such as an SREBP or SCAP.

In some embodiments of the uses and methods of treating insulin resistance, hyperglycemia, diabetes mellitus, obesity, or Metabolic Syndrome provided herein, the subject is administered between about 0.01 mg/kg to about 100 mg/kg of compound of Formula (I) or Formula (II), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

In other embodiments, the metabolic disorder is dyslipidemia. Thus, in other embodiments, provided herein are uses and methods of treating dyslipidemia in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. Dyslipidemia refers to abnormal blood plasma levels of one or more lipids or one or more lipoproteins, or any combinations thereof. Dyslipidemia may include depressed levels or elevated levels of one or more lipids and/or one or more lipoproteins, or a combination of depressed and elevated levels (for example, elevated levels of one type of lipid and depressed levels of another type of lipid and/or lipoprotein). Dyslipidemia may include, but is not limited to, elevated low density lipoprotein cholesterol (LDL), elevated apolipoprotein B, elevated triglycerides (TGs), elevated lipoprotein(a), elevated apolipoprotein A, reduced high density lipoprotein cholesterol (HDL), or reduced apolipoprotein A1, or any combinations thereof. Dyslipidemia, such as abnormal cholesterol or abnormal TG levels, is associated with an increased risk for vascular disease (such as heart attack or stroke), atherosclerosis, and coronary artery disease. In some embodiments of the uses and methods provided herein, the dyslipidemia is hyperlipidemia. Hyperlipidemia refers to the presence of an abnormally elevated level of lipids in the blood, and may include (1) hypercholesterolemia (an elevated cholesterol level); (2) hypertriglyceridemia, (an elevated triglyceride level); and (3) combined hyperlipidemia, (a combination of hypercholesterolemia and hypertriglyceridemia). Dyslipidemia may arise from a combination of genetic predisposition and diet, and may be associated with being overweight, diabetes, or Metabolic Syndrome. Lipid disorders may also arise as the result of certain medications (such as those used for anti-rejection regimens in people who have had organ or tissue transplants). In some embodiments, the dyslipidemia, such as hyperlipidemia, is mediated by a component of the SREBP pathway, such as an SREBP or SCAP. Thus, in some embodiments, provided herein are uses and methods of reducing cholesterol levels, modulating cholesterol metabolism, modulating cholesterol catabolism, modulating the absorption of dietary cholesterol, reversing cholesterol transport, or lowering triglycerides in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient.

In some embodiments of the uses and methods of treating dyslipidemia provided herein, such as reducing cholesterol levels, modulating cholesterol metabolism, modulating cholesterol catabolism, modulating the absorption of dietary cholesterol, reversing cholesterol transport, or lowering triglycerides in a subject in need thereof as provided herein, the subject is administered between about 0.01 mg/kg to about 100 mg/kg of compound of Formula (I) or Formula (II), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

In still other embodiments, provided herein are uses and methods of treating adiposopathy in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. In some embodiments, the adiposopathy is associated with Metabolic Syndrome. In some embodiments, the adiposopathy is mediated by a component of the SREBP pathway, such as an SREBP or SCAP.

In certain embodiments, provided herein are uses and methods of treating gallstones in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. Gallstones may be associated with gallbladder inflammation, pancreas inflammation, or liver inflammation. In certain embodiments, the gallstones are cholesterol gallstones, which may form when bile contains a high concentration of cholesterol and not enough bile salts. In some embodiments, the gallstones, which may include cholesterol gallstone disease, is mediated by a component of the SREBP pathway, such as an SREBP or SCAP.

In other embodiments, the disorder is pancreatitis. In yet other embodiments, the disorder is endotoxic shock, systemic inflammation, or xanthoma. In still further embodiments, the disorder is atherosclerosis, coronary artery disease, angina pectoris, carotid artery disease, stroke, or cerebral arteriosclerosis. In certain embodiments, any of the foregoing disorders are mediated by a component of the SREBP pathway, such as an SREBP or SCAP.

In some embodiments of the uses and methods of treating gall stones, pancreatitis, endotoxic shock, systemic inflammation, xanthoma, atherosclerosis, coronary artery disease, angina pectoris, carotid artery disease, stroke, or cerebral arteriosclerosis provided herein, the subject is administered between about 0.01 mg/kg to about 100 mg/kg of compound of Formula (I) or Formula (II), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

In some embodiments of any of the above embodiments, the subject is overweight, obese, has insulin resistance, is pre-diabetic or has type II diabetes. In certain embodiments of any of the preceding embodiments, the subject has NASH.

2. Hyperproliferative Disorders

In another embodiment, the disorder is a hyperproliferative disorder. Thus, in some embodiments, provided herein are uses and methods of treating a hyperproliferative disorder in a subject in need thereof, comprising administering to the subject in need thereof a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient.

As described above, the metabolism of fatty acids, cholesterol, and triglycerides may play a role in hyperproliferative disorders, such as cancer. Often, during transformation of non-cancerous cells to cancerous cell, cell metabolism shifts from catabolic to anabolic processes. Depending on the type of tumor, the tumor cells may synthesize up to 95% of the saturated and mono-unsaturated fatty acids. Some cancers exhibit increased synthesis of fatty acids and other lipids (such as cholesterol), and steroids (such as androgens). Elevated fatty acid synthase (FAS) expression may induce progression to S phase in cancer cells, and inhibition of FAS expression may reduce cell growth and may induce apoptosis. Thus, components of the SREBP pathway may play a role in hyperproliferative disorders.

Hyperproliferative disorders, which are disorders associated with some degree of abnormal cell proliferation, may be benign or malignant. Benign hyperproliferative disorders may include pre-cancerous disorders.

In some embodiments of the uses and methods provided herein, the disorder is a benign hyperproliferative disorder. In some embodiments, the benign hyperproliferative disorder is mediated by a component of the SREBP pathway, such as an SREBP or SCAP. In other embodiments, the disorder is a malignant hyperproliferative disorder. In some embodiments, the malignant hyperproliferative disorder is mediated by a component of the SREBP pathway, such as an SREBP or SCAP.

In some embodiments, the hyperproliferative disorder is breast cancer, liver cancer, ovarian cancer, pancreatic cancer, or prostate cancer.

In some embodiments of the uses and methods of treating a hyperproliferative disorder in a subject in need thereof, as described herein, between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof, relative to the body mass of the subject, is administered to the subject in need thereof. In certain embodiments, the compound or pharmaceutically acceptable salt, solvate, tautomer, isotope, or isomer thereof, is administered as a pharmaceutical composition, as described herein.

### III. Dosing and Methods of Administration

The dose of a compound of Formula (I) or Formula (II), or a pharmaceutically acceptable salt, solvate, tautomer, isotope, or isomer thereof, administered to a subject in need thereof according to any of the disclosed methods may vary with the particular compound or pharmaceutically acceptable salt, solvate, tautomer, isotope, or isomer thereof; the method of administration; the particular disorder being treated; and the characteristics of the subject (such as weight, sex, and/or age). In some embodiments, the amount of the compound or pharmaceutically acceptable salt, solvate, tautomer, isotope, or isomer thereof is a therapeutically effective amount.

The effective amount of the compound or pharmaceutically acceptable salt, solvate, tautomer, isotope, or isomer thereof, relative to the subject's body mass, may in some embodiments be between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of the compound of Formula (I) or Formula (II), or a pharmaceutically acceptable salt, solvate, tautomer, isotope, or isomer thereof is administered to a subject in need thereof. In certain embodiments, the compound or pharmaceutically acceptable salt, solvate, tautomer, isotope, or isomer thereof, is administered as a pharmaceutical composition, as described herein.

Any of the uses and methods provided herein may comprise administering to a subject in need therein a pharmaceutical composition that comprises an effective amount of a compound provided herein, such as a compound of Formula (I) or Formula (II), or a corresponding amount of a pharmaceutically acceptable salt, solvate, tautomer, isotope, or isomer thereof, and a pharmaceutically acceptable excipient.

The compounds of Formula (I) or Formula (II), or a pharmaceutically acceptable salt, solvate, tautomer, isotope, or isomer thereof as provided herein, or a pharmaceutical composition comprising any of these and a pharmaceutically acceptable excipient as provided herein, may be administered to a subject via any suitable route, including, for example, intravenous, intramuscular, subcutaneous, oral, or transdermal routes.

In certain embodiments, the present disclosure provides a method of treating a disorder in subject in need thereof by parenterally administering to the subject in need thereof an effective amount of a compound of Formula (I) or Formula (II), or a pharmaceutically acceptable salt, solvate, tautomer, isotope, or isomer thereof as provided herein, or a pharmaceutical composition comprising an effective amount of any of the foregoing and a pharmaceutically acceptable excipient as provided herein. In some embodiments, the disorder is a hyperproliferative disorder. In certain embodiments, the hyperproliferative disorder is cancer. In other embodiments, the disorder is fatty liver disease. In certain embodiments, the disorder is NASH. In some embodiments, the route of administration is intravenous, intra-arterial, intramuscular, or subcutaneous. In some embodiments, the route of administration is transdermal.

In some embodiments, provided herein are pharmaceutical compositions comprising a compound of Formula (I) or Formula (II), or a pharmaceutically acceptable salt, solvate, tautomer, isotope, or isomer thereof, and a pharmaceutically acceptable excipient, for the use in treating a disorder as described herein. In some embodiments, the disorder is prevented, or the onset delayed, or the development delayed. In some embodiments, the disorder is a hyperproliferative disorder. In certain embodiments, the hyperproliferative disorder is cancer. In some embodiments, the disorder is fatty liver disease. In certain embodiments, the disorder is NASH. In certain embodiments, the composition comprises a pharmaceutical formulation, which is present in a one or more unit dosage forms, for example one, two, three, four, or more unit dosage forms.

### IV. Kits

Also provided are articles of manufacture comprising a compound of Formula (I) or Formula (II), or a pharmaceutically acceptable salt, solvate, tautomer, isotope, or isomer thereof, or pharmaceutical compositions comprising any of the foregoing, or unit dosages comprising any of these, as described herein in suitable packaging for use in the methods described herein. Suitable packaging may include, for example, vials, vessels, ampules, bottles, jars, flexible packaging, and the like. An article of manufacture may further be sterilized and/or be sealed kits.

Further provided herein are kits comprising a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient. The kits may be used in any of the uses and methods described herein. In some embodiments, the kit further comprises instructions. The kits may be used for any one or more of the uses described herein, and, accordingly, may contain instructions for the treatment of a hyperproliferative disorder (such as cancer), fatty liver disease, or NASH. The kits may comprise one or more containers. Each component (if there is more than one component) may be packaged in separate containers or some components may be combined in one container where cross-reactivity and shelf life permit.

The kits may be in unit dosage forms, bulk packages (e.g., multi-dose packages) or subunit doses. For example, kits may be provided that contain sufficient dosages of a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient, as disclosed herein and/or a second pharmaceutically active compound useful for a disorder detailed herein to provide effective treatment of a subject for an extended period, such as one week, 2 weeks, 3 weeks, 4 weeks, 6 weeks, 8 weeks, 3 months, 4 months, 5 months, 7 months, 8 months, 9 months, or more. Kits may also include multiple unit doses of a compound of Formula (I) or Formula (II), or a pharmaceutically acceptable salt, solvate, tautomer, isotope, or isomer thereof, or a pharmaceutical composition comprising any of the foregoing and a pharmaceutically acceptable excipient, and instructions for use, and be packaged in quantities sufficient for storage and use in pharmacies (e.g., hospital pharmacies or compounding pharmacies).

The kits may optionally include a set of instructions, generally written instructions, although electronic storage media (e.g., magnetic diskette or optical disk) containing instructions are also acceptable, relating to the use of component(s) of the uses and methods as described herein. The instructions included with the kit may include information as to the components and their administration to an individual.

### Exemplary Embodiments

Embodiment I-1. A compound of Formula (I):

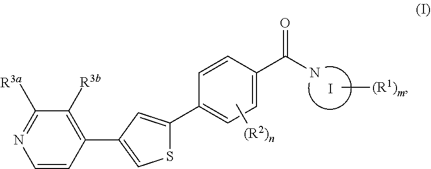

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, and (C₃-C₁₀)cycloalkyl, and wherein each heterocycloalkyl and cycloalkyl is unsubstituted or substituted with one or more substituents independently selected from the group consisting of halo and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

Embodiment I-2. The compound of Embodiment I-1, or a pharmaceutically acceptable salt, solvate, tautomer, isotope, or isomer thereof, wherein ring I is azetidinyl, pyrrolidinyl, or piperidinyl.

Embodiment I-3. The compound of Embodiment I-1, or a pharmaceutically acceptable salt, solvate, tautomer, isotope, or isomer thereof, wherein ring I is piperidinyl.

Embodiment I-4. The compound of any one of Embodiments I-1 to I-3, or a pharmaceutically acceptable salt, solvate, tautomer, isotope, or isomer thereof, wherein m is an integer from 1 to 4.

Embodiment I-5. The compound of any one of Embodiments I-1 to I-4, or a pharmaceutically acceptable salt, solvate, tautomer, isotope, or isomer thereof, wherein each R¹ is independently fluoro, —OH, unsubstituted alkyl, or alkyl substituted with one —OH.

Embodiment I-6. The compound of any one of Embodiments I-1 to I-5, or a pharmaceutically acceptable salt, solvate, tautomer, isotope, or isomer thereof, wherein n is 1.

Embodiment I-7. The compound of Embodiment I-6, or a pharmaceutically acceptable salt, solvate, tautomer, isotope, or isomer thereof, wherein R² is chloro.

Embodiment I-8. The compound of any one of Embodiments I-1 to I-7, or a pharmaceutically acceptable salt, solvate, tautomer, isotope, or isomer thereof, wherein R^{3a} is (C₃-C₁₀)cycloalkyl substituted with one or more substituents independently selected from the group consisting of halo and —OR⁵.

Embodiment I-9. The compound of Embodiment I-8, or a pharmaceutically acceptable salt, solvate, tautomer, isotope, or isomer thereof, wherein R^{3a} is (C₃-C₆)cycloalkyl substituted with one or more halo or —OH.

Embodiment I-10. The compound of any one of Embodiments I-1 to I-7, or a pharmaceutically acceptable salt, solvate, tautomer, isotope, or isomer thereof, wherein R^{3a} is (C₃-C₁₀)cycloalkyl substituted with one or more alkyl, wherein the cycloalkyl and each alkyl are independently unsubstituted or substituted with one or more substituents independently selected from the group consisting of halo and —OR⁶.

Embodiment I-11. The compound of Embodiment I-10, or a pharmaceutically acceptable salt, solvate, tautomer, isotope, or isomer thereof, wherein R^{3a} is (C₃-C₆)cycloalkyl substituted with one alkyl, wherein the alkyl is substituted with one —OH; and the cycloalkyl is not further substituted or is further substituted with one or two substituents independently selected from the group consisting of halo and —OR⁷.

Embodiment I-12. The compound of any one of Embodiments I-1 to I-7, or a pharmaceutically acceptable salt, solvate, tautomer, isotope, or isomer thereof, wherein R^{3a} is heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH.

Embodiment I-13. The compound of Embodiment I-12, or a pharmaceutically acceptable salt, solvate, tautomer, isotope, or isomer thereof, wherein the heterocycloalkyl is a 4- to 6-membered heterocycloalkyl comprising one or two heteroatoms.

Embodiment I-14. The compound of Embodiment I-13, or a pharmaceutically acceptable salt, solvate, tautomer, isotope, or isomer thereof, wherein each heteroatom is oxygen.

Embodiment I-15. The compound of Embodiment I-12 or I-13, or a pharmaceutically acceptable salt, solvate, tautomer, isotope, or isomer thereof, wherein the heterocycloalkyl is unsubstituted.

Embodiment I-16. The compound of any one of Embodiments I-12 to I-14, or a pharmaceutically acceptable salt, solvate, tautomer, isotope, or isomer thereof, wherein the heterocycloalkyl is substituted with —OH.

Embodiment I-17. The compound of any one of Embodiments I-1 to I-7, or a pharmaceutically acceptable salt, solvate, tautomer, isotope, or isomer thereof, wherein R^{3a} is —OR⁹, wherein R⁹ is heterocycloalkyl or (C₃-C₁₀)cycloalkyl, wherein the cycloalkyl is unsubstituted or substituted with one or more substituents independently selected from the group consisting of halo and —OH.

Embodiment I-18. The compound of Embodiment I-17, or a pharmaceutically acceptable salt, solvate, tautomer, isotope, or isomer thereof, wherein R⁹ is heterocycloalkyl.

Embodiment I-19. The compound of Embodiment I-17, or a pharmaceutically acceptable salt, solvate, tautomer, isotope, or isomer thereof, wherein R⁹ is (C₃₋₁₀)cycloalkyl.

Embodiment I-20. The compound of Embodiment I-17, or a pharmaceutically acceptable salt, solvate, tautomer, isotope, or isomer thereof, wherein R⁹ is (C₃₋₁₀)cycloalkyl, wherein the cycloalkyl is substituted with one or more substituents independently selected from the group consisting of halo and —OH.

Embodiment I-21. The compound of Embodiment I-17, or a pharmaceutically acceptable salt, solvate, tautomer, isotope, or isomer thereof, wherein R⁹ is alkyl, wherein the alkyl is unsubstituted or substituted with one or more substituents independently selected from the group consisting of —OH, halo, and (C₃-C₁₀)cycloalkyl.

Embodiment I-22. The compound of any one of Embodiments I-1 to I-7, or a pharmaceutically acceptable salt, solvate, tautomer, isotope, or isomer thereof, wherein R^{3a} is —CH₂—(C₃-C₁₀)cycloalkyl substituted with one or more substituents independently selected from the group consisting of halo and —OH.

Embodiment I-23. The compound of any one of Embodiments I-1 to I-7, or a pharmaceutically acceptable salt, solvate, tautomer, isotope, or isomer thereof, wherein R^{3b} together with R^{3a} and the atoms to which they are attached form a non-aromatic heterocyclyl group.

Embodiment I-24. The compound of Embodiment I-23, or a pharmaceutically acceptable salt, solvate, tautomer, isotope, or isomer thereof, wherein the non-aromatic heterocyclyl group is a 6-membered non-aromatic heterocyclyl group comprising two heteroatoms.

Embodiment I-25. The compound of Embodiment I-24, or a pharmaceutically acceptable salt, solvate, tautomer, isotope, or isomer thereof, wherein the two heteroatoms are independently O or N.

Embodiment I-26. The compound of any one of Embodiments I-23 to I-25, or a pharmaceutically acceptable salt, solvate, tautomer, isotope, or isomer thereof, wherein the non-aromatic heterocyclyl group is unsubstituted.

Embodiment I-27. The compound of Embodiment I-1, wherein the compound is:

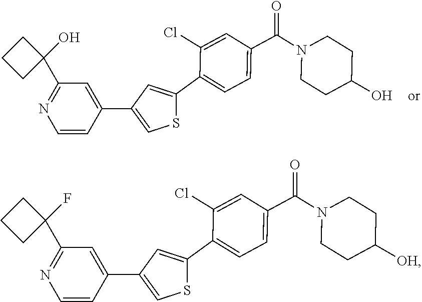 [SMILES: O=C(c1ccc(-c2cc(-c3ccnc(C4(O)CCC4)c3)cs2)c(Cl)c1)N1CCC(O)CC1 ; O=C(c1ccc(-c2cc(-c3ccnc(C4(F)CCC4)c3)cs2)c(Cl)c1)N1CCC(O)CC1]

or a pharmaceutically acceptable salt, solvate, tautomer, isotope, or isomer of any of the foregoing.

Embodiment I-28. The compound of Embodiment I-1, wherein the compound is:

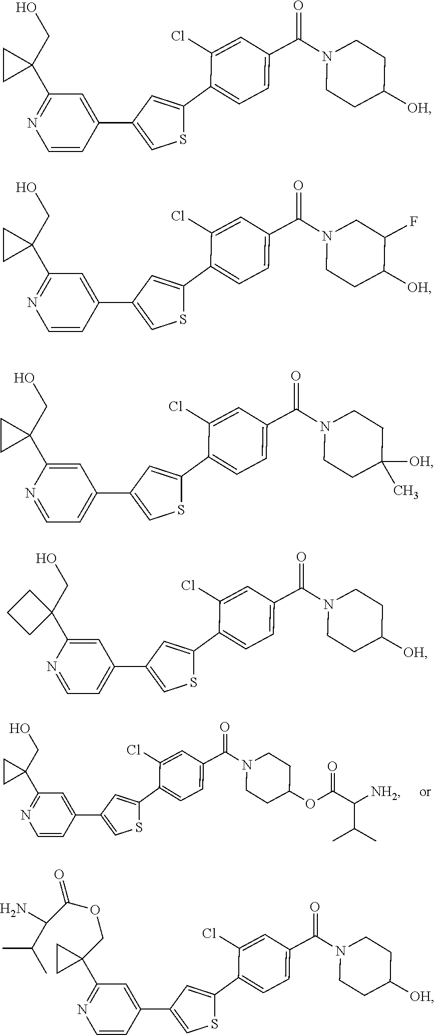

or a pharmaceutically acceptable salt, solvate, tautomer, isotope, or isomer thereof.

Embodiment I-29. The compound of Embodiment I-1, wherein the compound is:

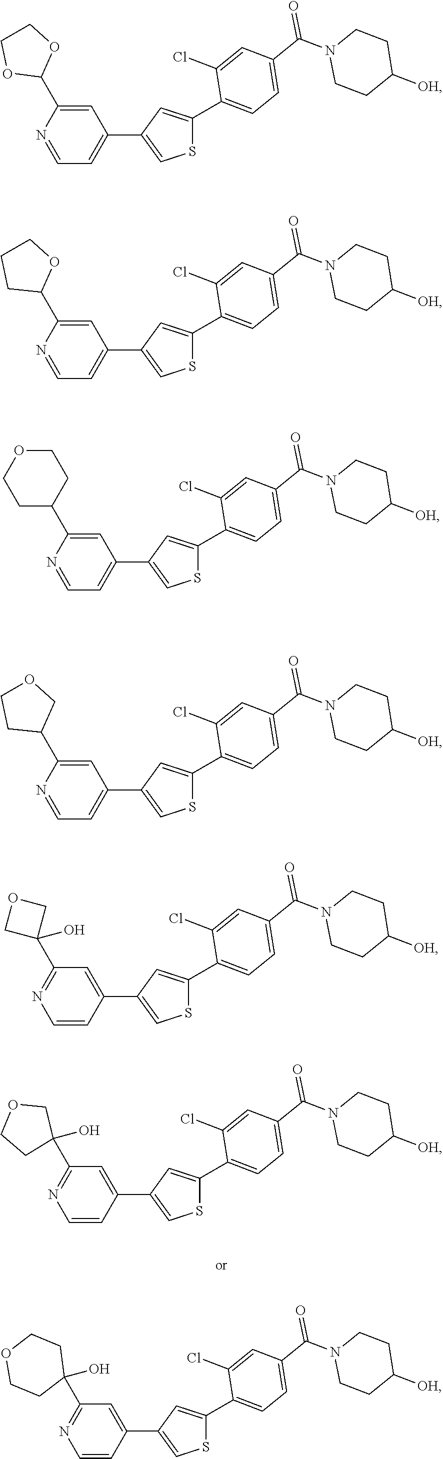

or a pharmaceutically acceptable salt, solvate, tautomer, isotope, or isomer of any of the foregoing.

Embodiment I-30. The compound of Embodiment I-1, wherein the compound is:

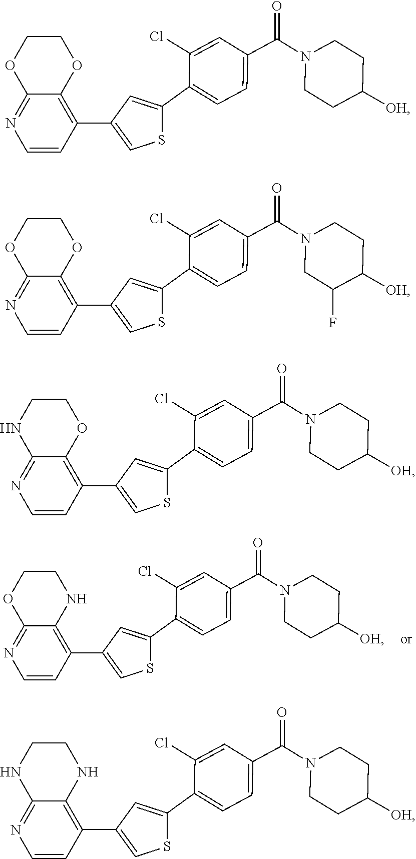 [SMILES: O=C(c1ccc(-c2cc(-c3ccnc4c3OCCO4)cs2)c(Cl)c1)N1CCC(O)CC1 ; O=C(c1ccc(-c2cc(-c3ccnc4c3OCCO4)cs2)c(Cl)c1)N1CCC(O)C(F)C1 ; O=C(c1ccc(-c2cc(-c3ccnc4c3OCCN4)cs2)c(Cl)c1)N1CCC(O)CC1 ; O=C(c1ccc(-c2cc(-c3ccnc4c3NCCO4)cs2)c(Cl)c1)N1CCC(O)CC1 ; O=C(c1ccc(-c2cc(-c3ccnc4c3NCCN4)cs2)c(Cl)c1)N1CCC(O)CC1]

or a pharmaceutically acceptable salt, solvate, tautomer, isotope, or isomer of any of the foregoing.

Embodiment I-31. The compound of Embodiment I-1, wherein the compound is:

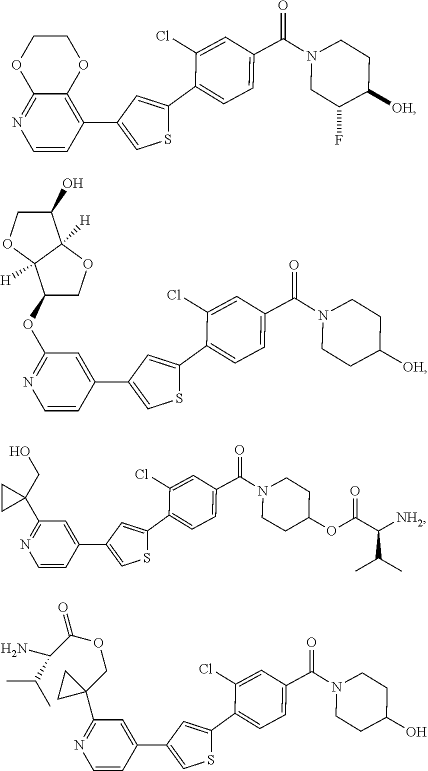

or a pharmaceutically acceptable salt, solvate, tautomer, isotope, or isomer of any of the foregoing.

Embodiment I-32. The compound of Embodiment I-1, wherein the compound is:

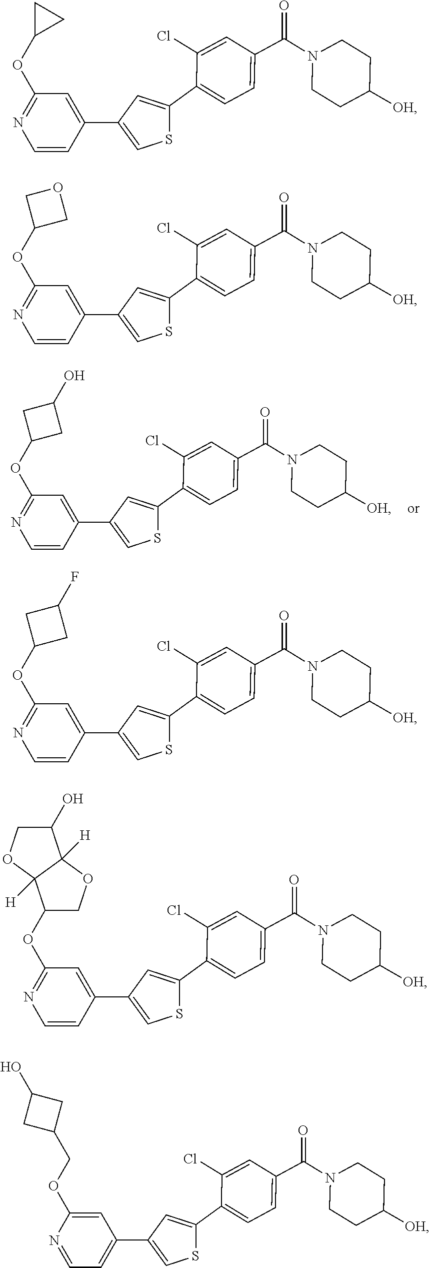

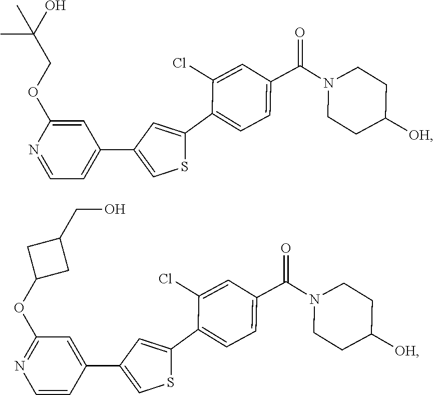

or a pharmaceutically acceptable salt, solvate, tautomer, isotope, or isomer of any of the foregoing.

Embodiment I-33. The compound of Embodiment I-1, wherein the compound is:

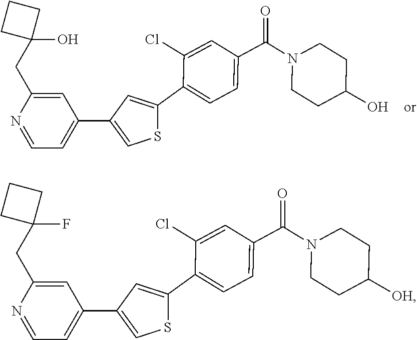

or a pharmaceutically acceptable salt, solvate, tautomer, isotope, or isomer of any of the foregoing.

Embodiment I-34. A pharmaceutical composition, comprising the compound of any one of Embodiments I-1 to I-33, or a pharmaceutically acceptable salt, solvate, tautomer, isotope, or isomer thereof, and a pharmaceutically acceptable excipient.

Embodiment I-35. A method of inhibiting a sterol regulatory element-binding protein (SREBP), comprising contacting the SREBP or contacting an SREBP cleavage activating-protein (SCAP) with an effective amount of a compound of any one of Embodiments I-1 to I-33, or a pharmaceutically acceptable salt, solvate, tautomer, isotope, or isomer thereof, or the pharmaceutical composition of Embodiment I-34.

Embodiment I-36. A method of inhibiting the proteolytic activation of a sterol regulatory element-binding protein (SREBP), comprising contacting an SREBP cleavage activating-protein (SCAP) with an effective amount of a compound of any one of Embodiments I-1 to I-33, or a pharmaceutically acceptable salt, solvate, tautomer, isotope, or isomer thereof, or the pharmaceutical composition of Embodiment I-34.

Embodiment I-37. The method of Embodiment I-35 or I-36, wherein the SREBP is an SREBP-1.

Embodiment I-38. The method of Embodiment I-37, wherein the SREBP-1 is SREBP-1a.

Embodiment I-39. The method of Embodiment I-37, wherein the SREBP-1 is SREBP-1c.

Embodiment I-40. The method of Embodiment I-35 or I-36, wherein the SREBP is SREBP-2.

Embodiment I-41. The method of any one of Embodiments I-35 to I-40, wherein SREBP is inhibited in a subject in need thereof.

Embodiment I-42. The method of any one of Embodiments I-35 to I-41, wherein SCAP is inhibited in a subject in need thereof.

Embodiment I-43. The method of any one of Embodiments I-35 to I-42, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof, or the pharmaceutical composition.

Embodiment I-44. A method of treating a disorder in a subject in need thereof, comprising administering to the subject in need thereof an effective amount of a compound of any one of Embodiments I-1 to I-33, or a pharmaceutically acceptable salt, solvate, tautomer, isotope, or isomer thereof, or the pharmaceutical composition of Embodiment I-34.

Embodiment I-45. A method of treating a disorder in a subject in need thereof, wherein the disorder is mediated by a sterol regulatory element-binding protein (SREBP), comprising administering to the subject in need thereof an effective amount of a compound of any one of Embodiments I-1 to I-33, or a pharmaceutically acceptable salt, solvate, tautomer, isotope, or isomer thereof, or the pharmaceutical composition of Embodiment I-34.

Embodiment I-46. The method of Embodiment I-44 or I-45, wherein the disorder is Metabolic Syndrome, type 2 diabetes, obesity, liver disease, insulin resistance, adiposopathy, or dyslipidemia.

Embodiment I-47. The method of Embodiment I-46, wherein the dyslipidemia is hypertriglyceridemia or elevated cholesterol levels.

Embodiment I-48. The method of Embodiment I-46, wherein the liver disease is nonalcoholic steatohepatitis, liver fibrosis, or liver inflammation, or a combination thereof.

Embodiment I-49. The method of Embodiment I-44 or I-45, wherein the disorder is a hyperproliferative disorder.

Embodiment I-50. The method of Embodiment I-49, wherein the hyperproliferative disorder is cancer.

Embodiment I-51. The method of Embodiment I-50, wherein the cancer is breast cancer, liver cancer, ovarian cancer, pancreatic cancer, or prostate cancer.

Embodiment I-52. The method of Embodiment I-44 or I-45, wherein the disorder is endotoxic shock, systemic inflammation, or atherosclerosis.

Embodiment I-53. A compound of any one of Embodiments I-1 to I-33, or a pharmaceutically acceptable salt, solvate, tautomer, isotope, or isomer thereof, for use in inhibiting a sterol regulatory element-binding protein (SREBP).

Embodiment I-54. A compound of any one of Embodiments I-1 to I-33, or a pharmaceutically acceptable salt, solvate, tautomer, isotope, or isomer thereof, for use in inhibiting the proteolytic activation of a sterol regulatory element-binding protein (SREBP).

Embodiment I-55. The compound for use of Embodiment I-53 or I-54, wherein the SREBP is an SREBP-1.

Embodiment I-56. The compound for use of Embodiment I-55, wherein the SREBP-1 is SREBP-1a.

Embodiment I-57. The compound for use of Embodiment I-55, wherein the SREBP-1 is SREBP-1c.

Embodiment I-58. The compound for use of Embodiment I-53 or I-54, wherein the SREBP is SREBP-2.

Embodiment I-59. The compound for use of any one of Embodiments I-53 to I-58, wherein SREBP is inhibited in a subject in need thereof.

Embodiment I-60. The compound for use of any one of Embodiments I-53 to I-58, wherein SCAP is inhibited in a subject in need thereof.

Embodiment I-61. The compound for use of any one of Embodiments I-53 to I-60, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

Embodiment I-62. A compound of any one of Embodiments I-1 to I-33, or a pharmaceutically acceptable salt, solvate, tautomer, isotope, or isomer thereof, for use in treating a disorder in a subject in need thereof.

Embodiment I-63. A compound of any one of Embodiments I-1 to I-33, or a pharmaceutically acceptable salt, solvate, tautomer, isotope, or isomer thereof, for use in treating a disorder in a subject in need thereof, wherein the disorder is mediated by a sterol regulatory element-binding protein (SREBP).

Embodiment I-64. The compound for use of Embodiment I-62 or I-63, wherein the disorder is Metabolic Syndrome, type 2 diabetes, obesity, liver disease, insulin resistance, adiposopathy, or dyslipidemia.

Embodiment I-65. The compound for use of Embodiment I-64, wherein the dyslipidemia is hypertriglyceridemia or elevated cholesterol levels.

Embodiment I-66. The compound for use of Embodiment I-64, wherein the liver disease is nonalcoholic steatohepatitis, liver fibrosis, or liver inflammation, or a combination thereof.

Embodiment I-67. The compound for use of Embodiment I-62 or I-63, wherein the disorder is a hyperproliferative disorder.

Embodiment I-68. The compound for use of Embodiment I-67, wherein the hyperproliferative disorder is cancer.

Embodiment I-69. The compound for use of Embodiment I-68, wherein the cancer is breast cancer, liver cancer, ovarian cancer, pancreatic cancer, or prostate cancer.

Embodiment I-70. The compound for use of Embodiment I-62 or I-63, wherein the disorder is endotoxic shock, systemic inflammation, or atherosclerosis.

Embodiment I-71. Use compound of any one of Embodiments I-1 to I-33, or a pharmaceutically acceptable salt, solvate, tautomer, isotope, or isomer thereof, in the manufacture of a medicament for inhibiting a sterol regulatory element-binding protein (SREBP).

Embodiment I-72. Use of a compound of any one of Embodiments I-1 to I-33, or a pharmaceutically acceptable salt, solvate, tautomer, isotope, or isomer thereof, in the manufacture of a medicament for inhibiting the proteolytic activation of a sterol regulatory element-binding protein (SREBP).

Embodiment I-73. The use of Embodiment I-71 or I-72, wherein the SREBP is an SREBP-1.

Embodiment I-74. The use of Embodiment I-73, wherein the SREBP-1 is SREBP-1a.

Embodiment I-75. The use of Embodiment I-73, wherein the SREBP-1 is SREBP-1c.

Embodiment I-76. The use of Embodiment I-71 or I-72, wherein the SREBP is SREBP-2.

Embodiment I-77. The use of any one of Embodiments I-71 to I-76, wherein SREBP is inhibited in a subject in need thereof.

Embodiment I-78. The use of any one of Embodiments I-71 to I-77, wherein SCAP is inhibited in a subject in need thereof.

Embodiment I-79. The use of any one of Embodiments I-71 to I-78, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

Embodiment I-80. Use of a compound of any one of Embodiments I-1 to I-33, or a pharmaceutically acceptable salt, solvate, tautomer, isotope, or isomer thereof, in the manufacture of a medicament for treating a disorder in a subject in need thereof.

Embodiment I-81. Use of a compound of any one of Embodiments I-1 to I-33, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

Embodiment I-82. The use of Embodiment I-80 or I-81, wherein the disorder is Metabolic Syndrome, type 2 diabetes, obesity, liver disease, insulin resistance, adiposopathy, or dyslipidemia.

Embodiment I-83. The use of Embodiment I-82, wherein the dyslipidemia is hypertriglyceridemia or elevated cholesterol levels.

Embodiment I-84. The use of Embodiment I-82, wherein the liver disease is nonalcoholic steatohepatitis, liver fibrosis, or liver inflammation, or a combination thereof.

Embodiment I-85. The use of Embodiment I-80 or I-81, wherein the disorder is a hyperproliferative disorder.

Embodiment I-86. The use of Embodiment I-85, wherein the hyperproliferative disorder is cancer.

Embodiment I-87. The use of Embodiment I-86, wherein the cancer is breast cancer, liver cancer, ovarian cancer, pancreatic cancer, or prostate cancer.

Embodiment I-88. The use of Embodiment I-80 or I-81, wherein the disorder is endotoxic shock, systemic inflammation, or atherosclerosis.

Embodiment II-1. A compound of Formula (I):

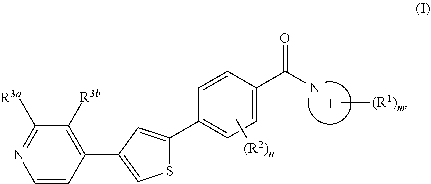

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
(C₃-C₁₀)cycloalkyl substituted with one or more substituents independently selected from the group consisting of halo and —OR⁵;
(C₃-C₁₀)cycloalkyl substituted with one or more alkyl, wherein the cycloalkyl and each alkyl are independently unsubstituted or substituted with one or more substituents independently selected from the group consisting of halo and —OR⁶;
heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
—OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, and (C₃-C₁₀)cycloalkyl, and wherein each heterocycloalkyl and cycloalkyl is unsubstituted or substituted with one or more substituents independently selected from the group consisting of halo and —OH;
CH₂—(C₃-C₁₀)cycloalkyl substituted with one or more substituents independently selected from the group consisting of halo and —OH; or
R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

Embodiment II-2. The compound of Embodiment II-1, or a pharmaceutically acceptable salt, solvate, tautomer, isotope, or isomer thereof, wherein ring I is azetidinyl, pyrrolidinyl, or piperidinyl.

Embodiment II-3. The compound of Embodiment II-1, or a pharmaceutically acceptable salt, solvate, tautomer, isotope, or isomer thereof, wherein ring I is piperidinyl.

Embodiment II-4. The compound of any one of Embodiments II-1 to II-3, or a pharmaceutically acceptable salt, solvate, tautomer, isotope, or isomer thereof, wherein m is an integer from 1 to 4.

Embodiment II-5. The compound of any one of Embodiments II-1 to II-4, or a pharmaceutically acceptable salt, solvate, tautomer, isotope, or isomer thereof, wherein each R¹ is independently fluoro, —OH, unsubstituted alkyl, or alkyl substituted with one —OH.

Embodiment II-6. The compound of any one of Embodiments II-1 to II-5, or a pharmaceutically acceptable salt, solvate, tautomer, isotope, or isomer thereof, wherein n is 1.

Embodiment II-7. The compound of Embodiment II-6, or a pharmaceutically acceptable salt, solvate, tautomer, isotope, or isomer thereof, wherein R² is chloro.

Embodiment II-8. The compound of any one of Embodiments II-1 to II-7, or a pharmaceutically acceptable salt, solvate, tautomer, isotope, or isomer thereof, wherein R^{3a} is (C₃-C₁₀)cycloalkyl substituted with one or more substituents independently selected from the group consisting of halo and —OR⁵.

Embodiment II-9. The compound of Embodiment II-8, or a pharmaceutically acceptable salt, solvate, tautomer, isotope, or isomer thereof, wherein R^{3a} is (C₃-C₆)cycloalkyl substituted with one or more halo or —OH.

Embodiment II-10. The compound of any one of Embodiments II-1 to II-7, or a pharmaceutically acceptable salt, solvate, tautomer, isotope, or isomer thereof, wherein R^{3a} is (C₃-C₁₀)cycloalkyl substituted with one or more alkyl, wherein the cycloalkyl and each alkyl are independently unsubstituted or substituted with one or more substituents independently selected from the group consisting of halo and —OR⁶.

Embodiment II-11. The compound of Embodiment II-10, or a pharmaceutically acceptable salt, solvate, tautomer, isotope, or isomer thereof, wherein R^{3a} is (C₃-C₆)cycloalkyl substituted with one alkyl, wherein the alkyl is substituted with one —OH; and the cycloalkyl is not further substituted or is further substituted with one or two substituents independently selected from the group consisting of halo and —OR⁷.

Embodiment II-12. The compound of any one of Embodiments II-1 to II-7, or a pharmaceutically acceptable salt, solvate, tautomer, isotope, or isomer thereof, wherein R^{3a} is heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH.

Embodiment II-13. The compound of Embodiment II-12, or a pharmaceutically acceptable salt, solvate, tautomer, isotope, or isomer thereof, wherein the heterocycloalkyl is a 4- to 6-membered heterocycloalkyl comprising one or two heteroatoms.

Embodiment II-14. The compound of Embodiment II-13, or a pharmaceutically acceptable salt, solvate, tautomer, isotope, or isomer thereof, wherein each heteroatom is oxygen.

Embodiment II-15. The compound of Embodiment II-12 or II-13, or a pharmaceutically acceptable salt, solvate, tautomer, isotope, or isomer thereof, wherein the heterocycloalkyl is unsubstituted.

Embodiment II-16. The compound of any one of Embodiments II-12 to II-14, or a pharmaceutically acceptable salt, solvate, tautomer, isotope, or isomer thereof, wherein the heterocycloalkyl is substituted with —OH.

Embodiment II-17. The compound of any one of Embodiments II-1 to II-7, or a pharmaceutically acceptable salt, solvate, tautomer, isotope, or isomer thereof, wherein R^{3a} is —OR⁹, wherein R⁹ is heterocycloalkyl or (C₃-C₁₀)cycloalkyl, wherein the cycloalkyl is unsubstituted or substituted with one or more substituents independently selected from the group consisting of halo and —OH.

Embodiment II-18. The compound of Embodiment II-17, or a pharmaceutically acceptable salt, solvate, tautomer, isotope, or isomer thereof, wherein R⁹ is heterocycloalkyl.

Embodiment II-19. The compound of Embodiment II-17, or a pharmaceutically acceptable salt, solvate, tautomer, isotope, or isomer thereof, wherein R⁹ is (C₃₋₁₀)cycloalkyl.

Embodiment II-20. The compound of Embodiment II-17, or a pharmaceutically acceptable salt, solvate, tautomer, isotope, or isomer thereof, wherein R⁹ is (C₃₋₁₀)cycloalkyl, wherein the cycloalkyl is substituted with one or more substituents independently selected from the group consisting of halo and —OH.

Embodiment II-21. The compound of Embodiment II-17, or a pharmaceutically acceptable salt, solvate, tautomer, isotope, or isomer thereof, wherein R⁹ is alkyl, wherein the alkyl is unsubstituted or substituted with one or more substituents independently selected from the group consisting of —OH, halo, and (C₃-C₁₀)cycloalkyl.

Embodiment II-22. The compound of any one of Embodiments II-1 to II-7, or a pharmaceutically acceptable salt, solvate, tautomer, isotope, or isomer thereof, wherein R^{3a} is —CH₂—(C₃-C₁₀)cycloalkyl substituted with one or more substituents independently selected from the group consisting of halo and —OH.

Embodiment II-23. The compound of any one of Embodiments II-1 to II-7, or a pharmaceutically acceptable salt, solvate, tautomer, isotope, or isomer thereof, wherein R^{3b} together with R^{3a} and the atoms to which they are attached form a non-aromatic heterocyclyl group.

Embodiment II-24. The compound of Embodiment II-23, or a pharmaceutically acceptable salt, solvate, tautomer, isotope, or isomer thereof, wherein the non-aromatic heterocyclyl group is a 6-membered non-aromatic heterocyclyl group comprising two heteroatoms.

Embodiment II-25. The compound of Embodiment II-24, or a pharmaceutically acceptable salt, solvate, tautomer, isotope, or isomer thereof, wherein the two heteroatoms are independently O or N.

Embodiment II-26. The compound of any one of Embodiments II-23 to II-25, or a pharmaceutically acceptable salt, solvate, tautomer, isotope, or isomer thereof, wherein the non-aromatic heterocyclyl group is unsubstituted.

Embodiment II-27. The compound of Embodiment II-1, wherein the compound is:

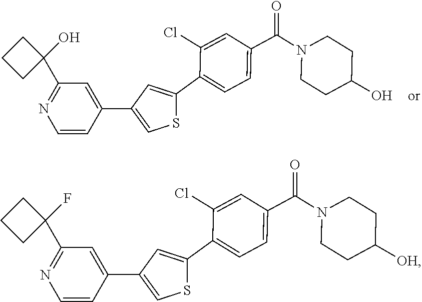 [SMILES: O=C(c1ccc(-c2cc(-c3ccnc(C4(O)CCC4)c3)cs2)c(Cl)c1)N1CCC(O)CC1 ; O=C(c1ccc(-c2cc(-c3ccnc(C4(F)CCC4)c3)cs2)c(Cl)c1)N1CCC(O)CC1]

or a pharmaceutically acceptable salt, solvate, tautomer, isotope, or isomer of any of the foregoing.

Embodiment II-28. The compound of Embodiment II-1, wherein the compound is:

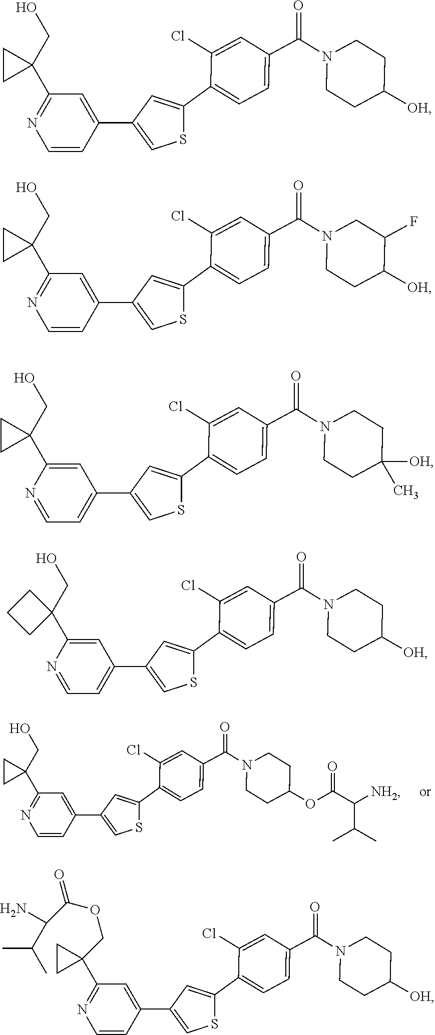

or a pharmaceutically acceptable salt, solvate, tautomer, isotope, or isomer thereof.

Embodiment II-29. The compound of Embodiment II-1, wherein the compound is:

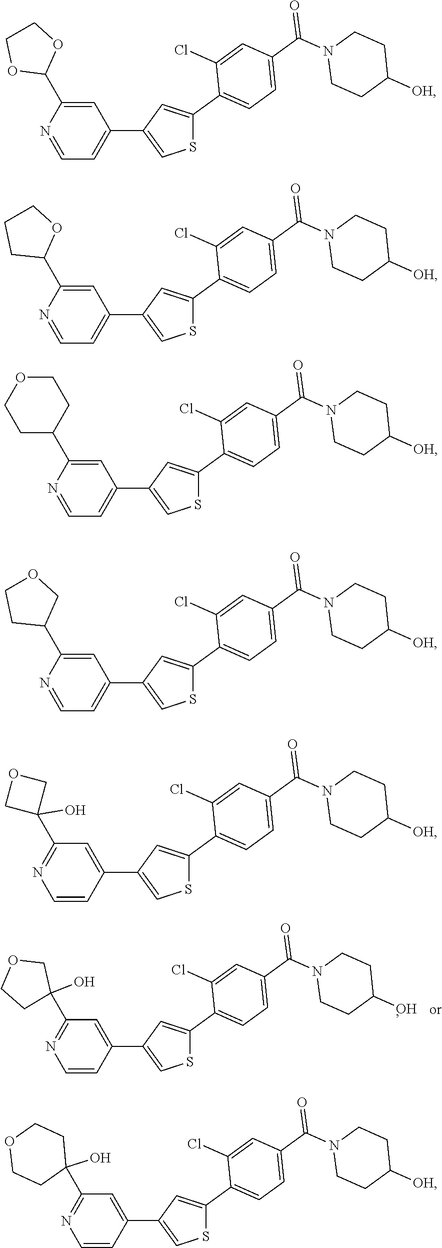

or a pharmaceutically acceptable salt, solvate, tautomer, isotope, or isomer of any of the foregoing.

Embodiment II-30. The compound of Embodiment II-1, wherein the compound is:

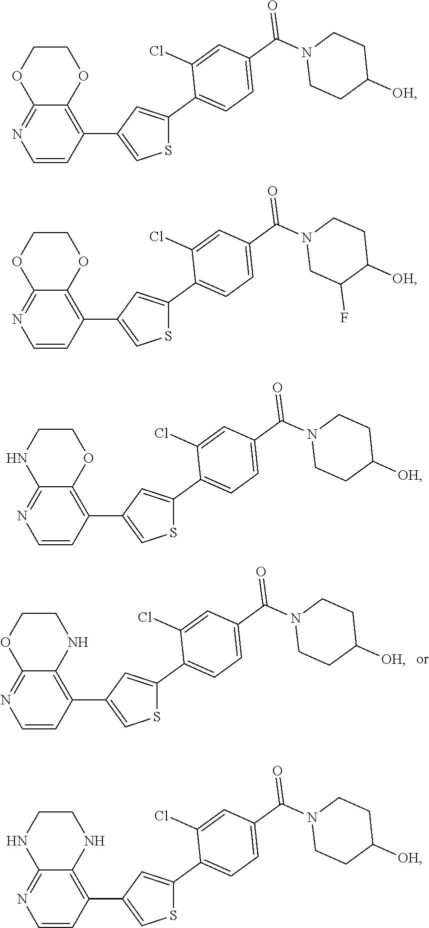 [SMILES: O=C(c1ccc(-c2cc(-c3ccnc4c3OCCO4)cs2)c(Cl)c1)N1CCC(O)CC1 ; O=C(c1ccc(-c2cc(-c3ccnc4c3OCCO4)cs2)c(Cl)c1)N1CCC(O)C(F)C1 ; O=C(c1ccc(-c2cc(-c3ccnc4c3OCCN4)cs2)c(Cl)c1)N1CCC(O)CC1 ; O=C(c1ccc(-c2cc(-c3ccnc4c3NCCO4)cs2)c(Cl)c1)N1CCC(O)CC1 ; O=C(c1ccc(-c2cc(-c3ccnc4c3NCCN4)cs2)c(Cl)c1)N1CCC(O)CC1]

or a pharmaceutically acceptable salt, solvate, tautomer, isotope, or isomer of any of the foregoing.

Embodiment II-31. The compound of Embodiment II-1, wherein the compound is:

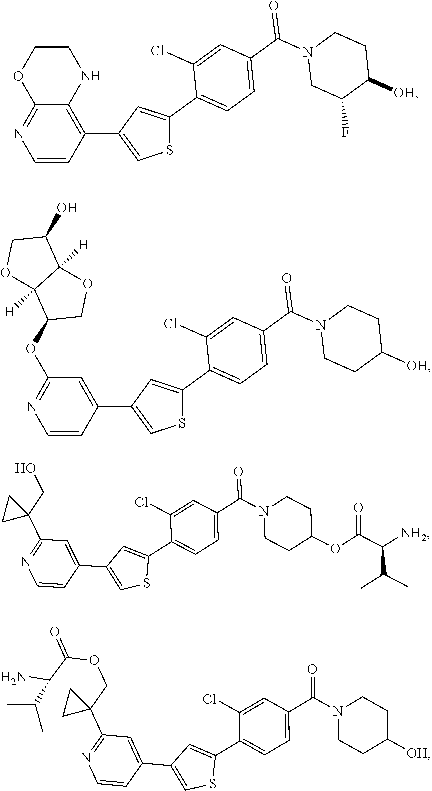

or a pharmaceutically acceptable salt, solvate, tautomer, isotope, or isomer of any of the foregoing.

Embodiment II-32. The compound of Embodiment II-1, wherein the compound is:

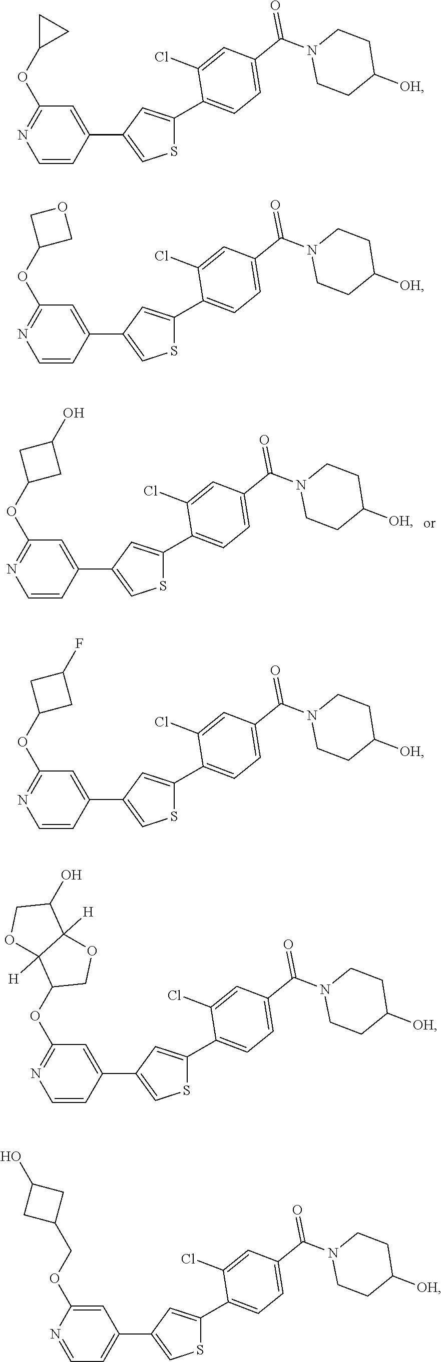

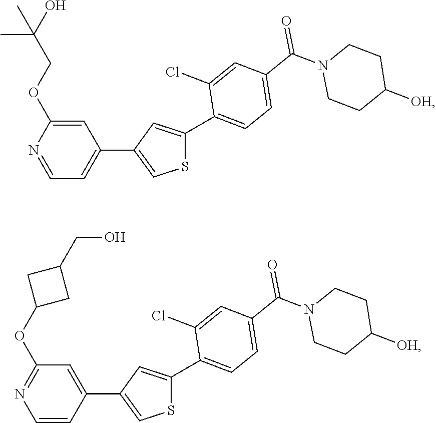 [SMILES: CC(C)(O)COc1cc(-c2csc(-c3ccc(C(=O)N4CCC(O)CC4)cc3Cl)c2)ccn1 ; O=C(c1ccc(-c2cc(-c3ccnc(OC4CC(CO)C4)c3)cs2)c(Cl)c1)N1CCC(O)CC1]

or a pharmaceutically acceptable salt, solvate, tautomer, isotope, or isomer of any of the foregoing.

Embodiment II-33. The compound of Embodiment II-1, wherein the compound is:

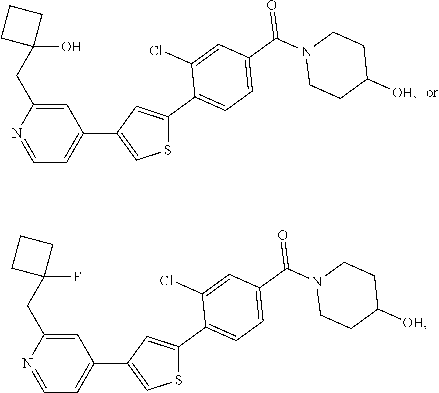 [SMILES: O=C(c1ccc(-c2cc(-c3ccnc(CC4(O)CCC4)c3)cs2)c(Cl)c1)N1CCC(O)CC1 ; O=C(c1ccc(-c2cc(-c3ccnc(CC4(F)CCC4)c3)cs2)c(Cl)c1)N1CCC(O)CC1]

or a pharmaceutically acceptable salt, solvate, tautomer, isotope, or isomer of any of the foregoing.

Embodiment II-34. The compound of Embodiment II-1, wherein the compound is:

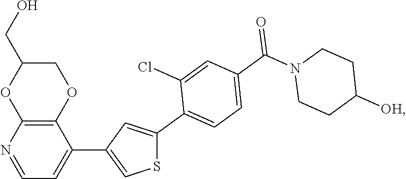 [SMILES: O=C(c1ccc(-c2cc(-c3ccnc4c3OCC(CO)O4)cs2)c(Cl)c1)N1CCC(O)CC1]

or a pharmaceutically acceptable salt, solvate, tautomer, isotope, or isomer of any of the foregoing.

Embodiment II-35. A pharmaceutical composition, comprising the compound of any one of Embodiments II-1 to II-34, or a pharmaceutically acceptable salt, solvate, tautomer, isotope, or isomer thereof, and a pharmaceutically acceptable excipient.

Embodiment II-36. A method of inhibiting a sterol regulatory element-binding protein (SREBP), comprising contacting the SREBP or contacting an SREBP cleavage activating-protein (SCAP) with an effective amount of a compound of any one of Embodiments II-1 to II-34, or a pharmaceutically acceptable salt, solvate, tautomer, isotope, or isomer thereof, or the pharmaceutical composition of Embodiment II-35.

Embodiment II-37. A method of inhibiting the proteolytic activation of a sterol regulatory element-binding protein (SREBP), comprising contacting an SREBP cleavage activating-protein (SCAP) with an effective amount of a compound of any one of Embodiments II-1 to II-34, or a pharmaceutically acceptable salt, solvate, tautomer, isotope, or isomer thereof, or the pharmaceutical composition of Embodiment II-35.

Embodiment II-38. The method of Embodiment II-36 or II-37, wherein the SREBP is an SREBP-1.

Embodiment II-39. The method of Embodiment II-38, wherein the SREBP-1 is SREBP-1a.

Embodiment II-40. The method of Embodiment II-38, wherein the SREBP-1 is SREBP-1c.

Embodiment II-41. The method of Embodiment II-36 or II-37, wherein the SREBP is SREBP-2.

Embodiment II-42. The method of any one of Embodiments II-36 to II-41, wherein SREBP is inhibited in a subject in need thereof.

Embodiment II-43. The method of any one of Embodiments II-36 to II-42, wherein SCAP is inhibited in a subject in need thereof.

Embodiment II-44. The method of any one of Embodiments II-36 to II-43, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof, or the pharmaceutical composition.

Embodiment II-45. A method of treating a disorder in a subject in need thereof, comprising administering to the subject in need thereof an effective amount of a compound of any one of Embodiments II-1 to II-34, or a pharmaceutically acceptable salt, solvate, tautomer, isotope, or isomer thereof, or the pharmaceutical composition of Embodiment II-35.

Embodiment II-46. A method of treating a disorder in a subject in need thereof, wherein the disorder is mediated by a sterol regulatory element-binding protein (SREBP), comprising administering to the subject in need thereof an effective amount of a compound of any one of Embodiments II-1 to II-34, or a pharmaceutically acceptable salt, solvate, tautomer, isotope, or isomer thereof, or the pharmaceutical composition of Embodiment II-35.

Embodiment II-47. The method of Embodiment II-45 or II-46, wherein the disorder is Metabolic Syndrome, type 2 diabetes, obesity, liver disease, insulin resistance, adiposopathy, or dyslipidemia.

Embodiment II-48. The method of Embodiment II-47, wherein the dyslipidemia is hypertriglyceridemia or elevated cholesterol levels.

Embodiment II-49. The method of Embodiment II-47, wherein the liver disease is nonalcoholic steatohepatitis, liver fibrosis, or liver inflammation, or a combination thereof.

Embodiment II-50. The method of Embodiment II-45 or II-46, wherein the disorder is a hyperproliferative disorder.

Embodiment II-51. The method of Embodiment II-50, wherein the hyperproliferative disorder is cancer.

Embodiment II-52. The method of Embodiment II-51, wherein the cancer is breast cancer, liver cancer, ovarian cancer, pancreatic cancer, or prostate cancer.

Embodiment II-53. The method of Embodiment II-45 or II-46, wherein the disorder is endotoxic shock, systemic inflammation, or atherosclerosis.

Embodiment II-54. A compound of any one of Embodiments II-1 to II-34, or a pharmaceutically acceptable salt, solvate, tautomer, isotope, or isomer thereof, for use in inhibiting a sterol regulatory element-binding protein (SREBP).

Embodiment II-55. A compound of any one of Embodiments II-1 to II-34, or a pharmaceutically acceptable salt, solvate, tautomer, isotope, or isomer thereof, for use in inhibiting the proteolytic activation of a sterol regulatory element-binding protein (SREBP).

Embodiment II-56. The compound for use of Embodiment II-54 or II-55, wherein the SREBP is an SREBP-1.

Embodiment II-57. The compound for use of Embodiment II-56, wherein the SREBP-1 is SREBP-1a.

Embodiment II-58. The compound for use of Embodiment II-56, wherein the SREBP-1 is SREBP-1c.

Embodiment II-59. The compound for use of Embodiment II-54 or II-55, wherein the SREBP is SREBP-2.

Embodiment II-60. The compound for use of any one of Embodiments II-54 to II-59, wherein SREBP is inhibited in a subject in need thereof.

Embodiment II-61. The compound for use of any one of Embodiments II-54 to II-59, wherein SCAP is inhibited in a subject in need thereof.

Embodiment II-62. The compound for use of any one of Embodiments II-54 to II-61, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

Embodiment II-63. A compound of any one of Embodiments II-1 to II-34, or a pharmaceutically acceptable salt, solvate, tautomer, isotope, or isomer thereof, for use in treating a disorder in a subject in need thereof.

Embodiment II-64. A compound of any one of Embodiments II-1 to II-34, or a pharmaceutically acceptable salt, solvate, tautomer, isotope, or isomer thereof, for use in treating a disorder in a subject in need thereof, wherein the disorder is mediated by a sterol regulatory element-binding protein (SREBP).

Embodiment II-65. The compound for use of Embodiment II-63 or II-64, wherein the disorder is Metabolic Syndrome, type 2 diabetes, obesity, liver disease, insulin resistance, adiposopathy, or dyslipidemia.

Embodiment II-66. The compound for use of Embodiment II-65, wherein the dyslipidemia is hypertriglyceridemia or elevated cholesterol levels.

Embodiment II-67. The compound for use of Embodiment II-65, wherein the liver disease is nonalcoholic steatohepatitis, liver fibrosis, or liver inflammation, or a combination thereof.

Embodiment II-68. The compound for use of Embodiment II-63 or II-64, wherein the disorder is a hyperproliferative disorder.

Embodiment II-69. The compound for use of Embodiment II-68, wherein the hyperproliferative disorder is cancer.

Embodiment II-70. The compound for use of Embodiment II-69, wherein the cancer is breast cancer, liver cancer, ovarian cancer, pancreatic cancer, or prostate cancer.

Embodiment II-71. The compound for use of Embodiment II-63 or II-64, wherein the disorder is endotoxic shock, systemic inflammation, or atherosclerosis.

Embodiment II-72. Use compound of any one of Embodiments II-1 to II-34, or a pharmaceutically acceptable salt, solvate, tautomer, isotope, or isomer thereof, in the manufacture of a medicament for inhibiting a sterol regulatory element-binding protein (SREBP).

Embodiment II-73. Use of a compound of any one of Embodiments II-1 to II-34, or a pharmaceutically acceptable salt, solvate, tautomer, isotope, or isomer thereof, in the manufacture of a medicament for inhibiting the proteolytic activation of a sterol regulatory element-binding protein (SREBP).

Embodiment II-74. The use of Embodiment II-72 or II-73, wherein the SREBP is an SREBP-1.

Embodiment II-75. The use of Embodiment II-74, wherein the SREBP-1 is SREBP-1a.

Embodiment II-76. The use of Embodiment II-74, wherein the SREBP-1 is SREBP-1c.

Embodiment II-76. The use of Embodiment II-72 or II-73, wherein the SREBP is SREBP-2.

Embodiment II-78. The use of any one of Embodiments II-72 to II-77, wherein SREBP is inhibited in a subject in need thereof.

Embodiment II-79. The use of any one of Embodiments II-72 to II-78, wherein SCAP is inhibited in a subject in need thereof.

Embodiment II-80. The use of any one of Embodiments II-72 to II-79, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

Embodiment II-81. Use of a compound of any one of Embodiments II-1 to II-34, or a pharmaceutically acceptable salt, solvate, tautomer, isotope, or isomer thereof, in the manufacture of a medicament for treating a disorder in a subject in need thereof.

Embodiment II-82. Use of a compound of any one of Embodiments II-1 to II-34, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

Embodiment II-83. The use of Embodiment II-81 or II-82, wherein the disorder is Metabolic Syndrome, type 2 diabetes, obesity, liver disease, insulin resistance, adiposopathy, or dyslipidemia.

Embodiment II-84. The use of Embodiment II-83, wherein the dyslipidemia is hypertriglyceridemia or elevated cholesterol levels.

Embodiment II-85. The use of Embodiment II-83, wherein the liver disease is nonalcoholic steatohepatitis, liver fibrosis, or liver inflammation, or a combination thereof.

Embodiment II-86. The use of Embodiment II-81 or II-82, wherein the disorder is a hyperproliferative disorder.

Embodiment II-87. The use of Embodiment II-86, wherein the hyperproliferative disorder is cancer.

Embodiment II-88. The use of Embodiment II-87, wherein the cancer is breast cancer, liver cancer, ovarian cancer, pancreatic cancer, or prostate cancer.

Embodiment II-89. The use of Embodiment II-81 or II-82, wherein the disorder is endotoxic shock, systemic inflammation, or atherosclerosis.

Embodiment III-1. A compound of Formula (II):

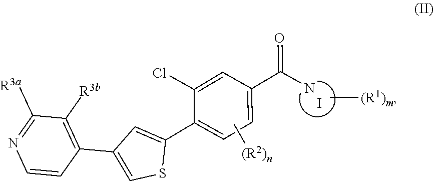

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, heterocycloalkyl, and (C₃-C₁₀)cycloalkyl, and wherein each heterocycloalkyl and cycloalkyl is unsubstituted or substituted with one or more substituents independently selected from the group consisting of alkyl, halo, and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

Embodiment III-2. A compound of Formula (I):

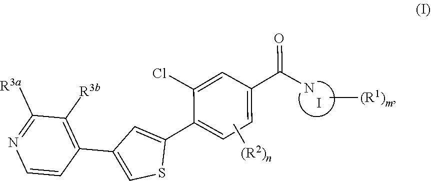

or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
   (C₃-C₁₀)cycloalkyl substituted with one or more substituents independently selected from the group consisting of halo and —OR⁵;
   (C₃-C₁₀)cycloalkyl substituted with one or more alkyl, wherein the cycloalkyl and each alkyl are independently unsubstituted or substituted with one or more substituents independently selected from the group consisting of halo and —OR⁶;
   heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
   —OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, and (C₃-C₁₀)cycloalkyl, and wherein each heterocycloalkyl and cycloalkyl is unsubstituted or substituted with one or more substituents independently selected from the group consisting of halo and —OH;
   —CH₂—(C₃-C₁₀)cycloalkyl substituted with one or more substituents independently selected from the group consisting of halo and —OH; or
   R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl; wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

Embodiment III-3. The compound of Embodiment III-1 or III-2, or a pharmaceutically acceptable salt, solvate, tautomer, isotope, or isomer thereof, wherein ring I is azetidinyl, pyrrolidinyl, or piperidinyl.

Embodiment III-4. The compound of Embodiment III-1 or III-2, or a pharmaceutically acceptable salt, solvate, tautomer, isotope, or isomer thereof, wherein ring I is piperidinyl.

Embodiment III-5. The compound of any one of Embodiments III-1 to III-4, or a pharmaceutically acceptable salt, solvate, tautomer, isotope, or isomer thereof, wherein m is an integer from 1 to 4.

Embodiment III-6. The compound of any one of Embodiments III-1 to III-5, or a pharmaceutically acceptable salt, solvate, tautomer, isotope, or isomer thereof, wherein each R¹ is independently fluoro, —OH, unsubstituted alkyl, or alkyl substituted with one —OH.

Embodiment III-7. The compound of any one of Embodiments III-1 to III-6, or a pharmaceutically acceptable salt, solvate, tautomer, isotope, or isomer thereof, wherein n is 1.

Embodiment III-8. The compound of Embodiment III-7, or a pharmaceutically acceptable salt, solvate, tautomer, isotope, or isomer thereof, wherein R² is chloro.

Embodiment III-9. The compound of any one of Embodiments III-1 to III-8, or a pharmaceutically acceptable salt, solvate, tautomer, isotope, or isomer thereof, wherein R^{3a} is (C₃-C₁₀)cycloalkyl substituted with one or more substituents independently selected from the group consisting of halo and —OR⁵.

Embodiment III-10. The compound of Embodiment III-8, or a pharmaceutically acceptable salt, solvate, tautomer, isotope, or isomer thereof, wherein R^{3a} is (C₃-C₆)cycloalkyl substituted with one or more halo or —OH.

Embodiment III-11. The compound of any one of Embodiments III-1 to III-8, or a pharmaceutically acceptable salt, solvate, tautomer, isotope, or isomer thereof, wherein R^{3a} is (C₃-C₁₀)cycloalkyl substituted with one or more alkyl, wherein the cycloalkyl and each alkyl are independently unsubstituted or substituted with one or more substituents independently selected from the group consisting of halo and —OR⁶.

Embodiment III-12. The compound of Embodiment III-11, or a pharmaceutically acceptable salt, solvate, tautomer, isotope, or isomer thereof, wherein R^{3a} is (C₃-C₆)cycloalkyl substituted with one alkyl, wherein the alkyl is substituted with one —OH; and the cycloalkyl is not further substituted or is further substituted with one or two substituents independently selected from the group consisting of halo and —OR⁷.

Embodiment III-13. The compound of any one of Embodiments III-1 to III-8, or a pharmaceutically acceptable salt, solvate, tautomer, isotope, or isomer thereof, wherein R^{3a} is heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH.

Embodiment III-14. The compound of Embodiment III-13, or a pharmaceutically acceptable salt, solvate, tautomer, isotope, or isomer thereof, wherein the heterocycloalkyl is a 4- to 6-membered heterocycloalkyl comprising one or two heteroatoms.

Embodiment III-15. The compound of Embodiment III-14, or a pharmaceutically acceptable salt, solvate, tautomer, isotope, or isomer thereof, wherein each heteroatom is oxygen.

Embodiment III-16. The compound of Embodiment III-13 or III-14, or a pharmaceutically acceptable salt, solvate, tautomer, isotope, or isomer thereof, wherein the heterocycloalkyl is unsubstituted.

Embodiment III-17. The compound of any one of Embodiments III-13 to III-15, or a pharmaceutically acceptable salt, solvate, tautomer, isotope, or isomer thereof, wherein the heterocycloalkyl is substituted with —OH.

Embodiment III-18. The compound of any one of Embodiments III-1 to III-8, or a pharmaceutically acceptable salt, solvate, tautomer, isotope, or isomer thereof, wherein R^{3a} is —OR⁹, wherein R⁹ is heterocycloalkyl or (C₃-C₁₀)cycloalkyl, wherein the cycloalkyl is unsubstituted or substituted with one or more substituents independently selected from the group consisting of halo and —OH.

Embodiment III-19. The compound of Embodiment III-18, or a pharmaceutically acceptable salt, solvate, tautomer, isotope, or isomer thereof, wherein R⁹ is heterocycloalkyl.

Embodiment III-20. The compound of Embodiment III-18, or a pharmaceutically acceptable salt, solvate, tautomer, isotope, or isomer thereof, wherein R⁹ is (C₃₋₁₀)cycloalkyl.

Embodiment III-21. The compound of Embodiment III-18, or a pharmaceutically acceptable salt, solvate, tautomer, isotope, or isomer thereof, wherein R⁹ is (C₃₋₁₀)cycloalkyl, wherein the cycloalkyl is substituted with one or more substituents independently selected from the group consisting of halo and —OH.

Embodiment III-22. The compound of Embodiment III-18, or a pharmaceutically acceptable salt, solvate, tautomer, isotope, or isomer thereof, wherein R⁹ is alkyl, wherein the alkyl is unsubstituted or substituted with one or more substituents independently selected from the group consisting of —OH, halo, and (C₃-C₁₀)cycloalkyl.

Embodiment III-23. The compound of any one of Embodiments III-1 to III-8, or a pharmaceutically acceptable salt, solvate, tautomer, isotope, or isomer thereof, wherein R^{3a} is —CH₂—(C₃-C₁₀)cycloalkyl substituted with one or more substituents independently selected from the group consisting of halo and —OH.

Embodiment III-24. The compound of any one of Embodiments III-1 to III-8, or a pharmaceutically acceptable salt, solvate, tautomer, isotope, or isomer thereof, wherein R^{3b} together with R^{3a} and the atoms to which they are attached form a non-aromatic heterocyclyl group.

Embodiment III-25. The compound of Embodiment III-24, or a pharmaceutically acceptable salt, solvate, tautomer, isotope, or isomer thereof, wherein the non-aromatic heterocyclyl group is a 6-membered non-aromatic heterocyclyl group comprising two heteroatoms.

Embodiment III-26. The compound of Embodiment III-25, or a pharmaceutically acceptable salt, solvate, tautomer, isotope, or isomer thereof, wherein the two heteroatoms are independently O or N.

Embodiment III-27. The compound of any one of Embodiments III-24 to III-26, or a pharmaceutically acceptable salt, solvate, tautomer, isotope, or isomer thereof, wherein the non-aromatic heterocyclyl group is unsubstituted.

Embodiment III-28. The compound of Embodiment III-1 or III-2, wherein the compound is:

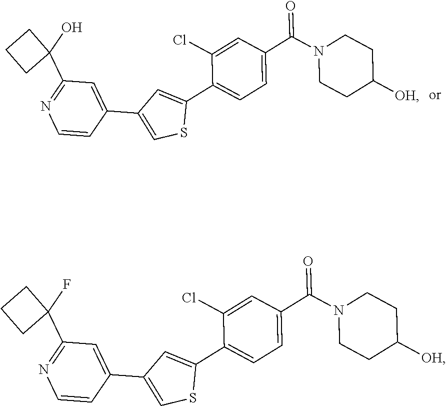 [SMILES: O=C(c1ccc(-c2cc(-c3ccnc(C4(O)CCC4)c3)cs2)c(Cl)c1)N1CCC(O)CC1 ; O=C(c1ccc(-c2cc(-c3ccnc(C4(F)CCC4)c3)cs2)c(Cl)c1)N1CCC(O)CC1]

or a pharmaceutically acceptable salt, solvate, tautomer, isotope, or isomer of any of the foregoing.

Embodiment III-29. The compound of Embodiment III-1 or III-2, wherein the compound is:

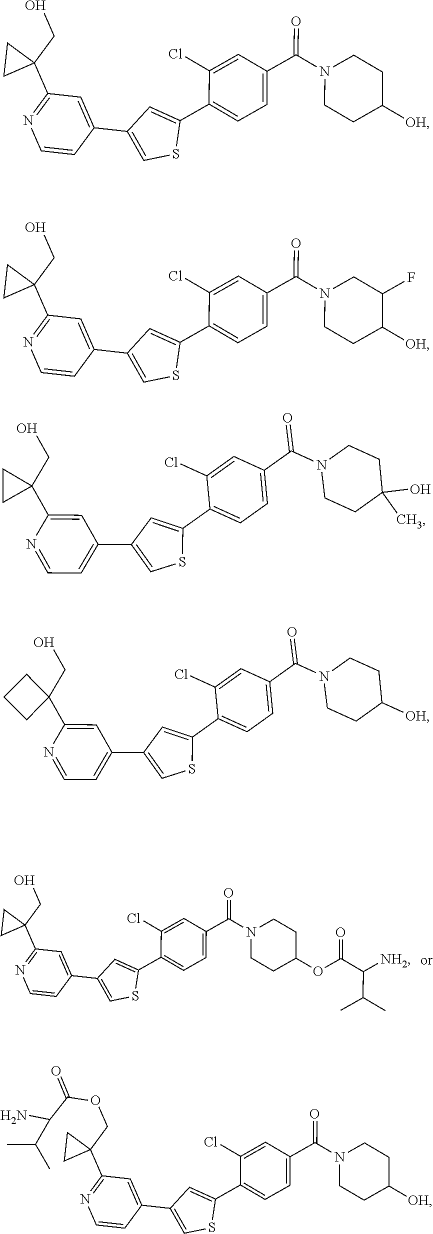 [SMILES: O=C(c1ccc(-c2cc(-c3ccnc(C4(CO)CC4)c3)cs2)c(Cl)c1)N1CCC(O)CC1 ; O=C(c1ccc(-c2cc(-c3ccnc(C4(CO)CC4)c3)cs2)c(Cl)c1)N1CCC(O)C(F)C1 ; CC1(O)CCN(C(=O)c2ccc(-c3cc(-c4ccnc(C5(CO)CC5)c4)cs3)c(Cl)c2)CC1 ; O=C(c1ccc(-c2cc(-c3ccnc(C4(CO)CCC4)c3)cs2)c(Cl)c1)N1CCC(O)CC1 ; CC(C)C(N)C(=O)OC1CCN(C(=O)c2ccc(-c3cc(-c4ccnc(C5(CO)CC5)c4)cs3)c(Cl)c2)CC1 ; CC(C)C(N)C(=O)OCC1(c2cc(-c3csc(-c4ccc(C(=O)N5CCC(O)CC5)cc4Cl)c3)ccn2)CC1]

or a pharmaceutically acceptable salt, solvate, tautomer, isotope, or isomer thereof.

Embodiment III-30. The compound of Embodiment III-1 or III-2, wherein the compound is:

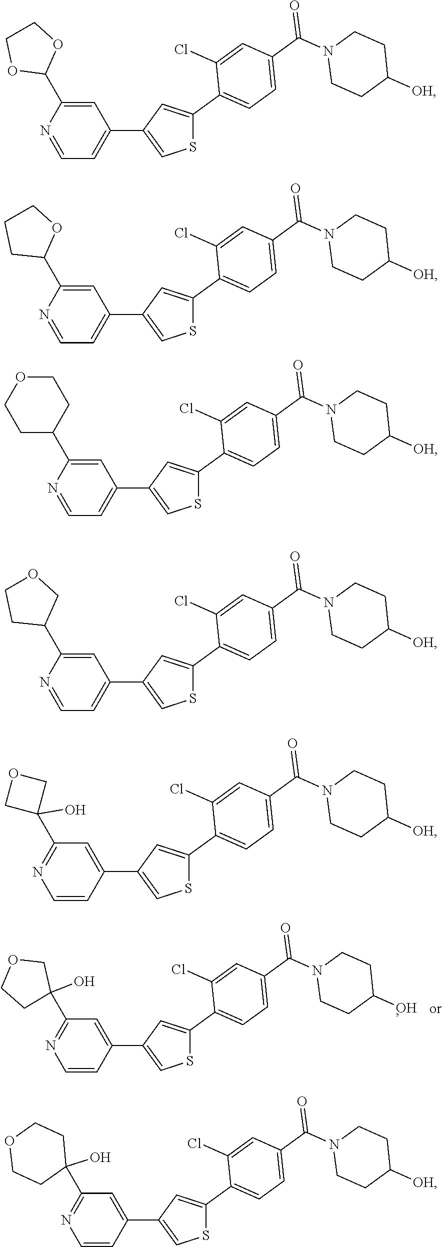

or a pharmaceutically acceptable salt, solvate, tautomer, isotope, or isomer of any of the foregoing.

Embodiment III-31. The compound of Embodiment III-1 or III-2, wherein the compound is:

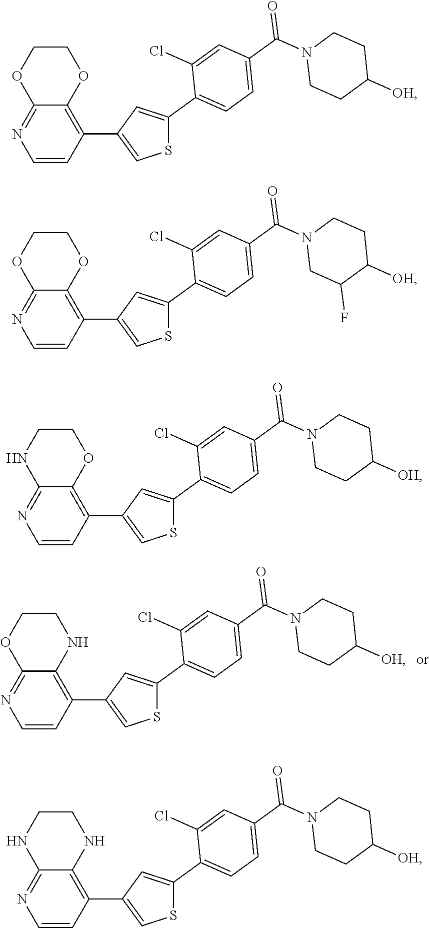 [SMILES: O=C(c1ccc(-c2cc(-c3ccnc4c3OCCO4)cs2)c(Cl)c1)N1CCC(O)CC1 ; O=C(c1ccc(-c2cc(-c3ccnc4c3OCCO4)cs2)c(Cl)c1)N1CCC(O)C(F)C1 ; O=C(c1ccc(-c2cc(-c3ccnc4c3OCCN4)cs2)c(Cl)c1)N1CCC(O)CC1 ; O=C(c1ccc(-c2cc(-c3ccnc4c3NCCO4)cs2)c(Cl)c1)N1CCC(O)CC1 ; O=C(c1ccc(-c2cc(-c3ccnc4c3NCCN4)cs2)c(Cl)c1)N1CCC(O)CC1]

or a pharmaceutically acceptable salt, solvate, tautomer, isotope, or isomer of any of the foregoing.

Embodiment III-32. The compound of Embodiment III-1 or III-2, wherein the compound is:

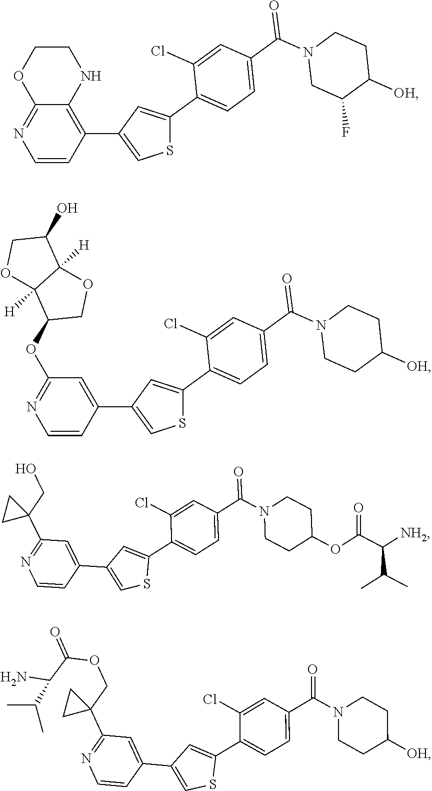

or a pharmaceutically acceptable salt, solvate, tautomer, isotope, or isomer of any of the foregoing.

Embodiment III-33. The compound of Embodiment III-1 or III-2, wherein the compound is:

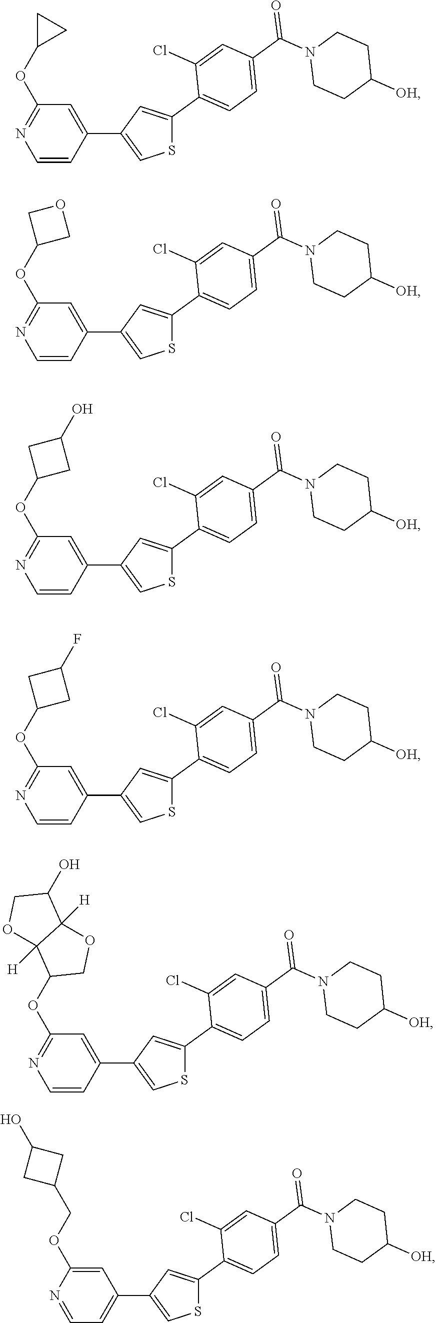

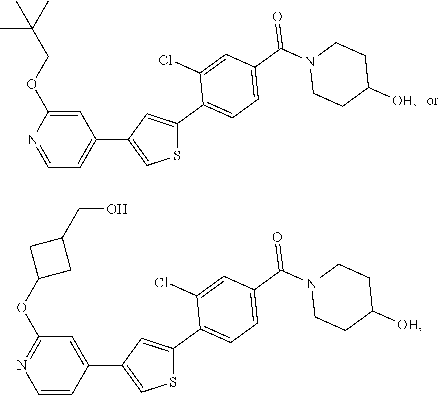

or a pharmaceutically acceptable salt, solvate, tautomer, isotope, or isomer of any of the foregoing.

Embodiment III-34. The compound of Embodiment III-1 or III-2, wherein the compound is:

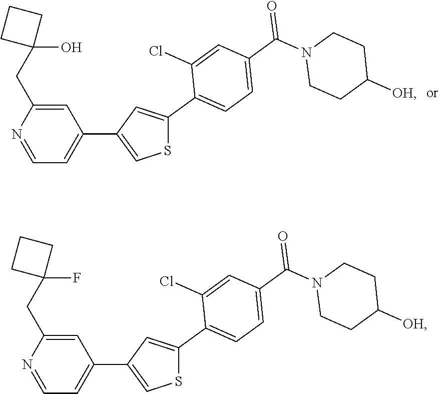 [SMILES: O=C(c1ccc(-c2cc(-c3ccnc(CC4(O)CCC4)c3)cs2)c(Cl)c1)N1CCC(O)CC1 ; O=C(c1ccc(-c2cc(-c3ccnc(CC4(F)CCC4)c3)cs2)c(Cl)c1)N1CCC(O)CC1]

or a pharmaceutically acceptable salt, solvate, tautomer, or isotope, or isomer of any of the foregoing.

Embodiment III-35. The compound of Embodiment III-1 or III-2, wherein the compound is:

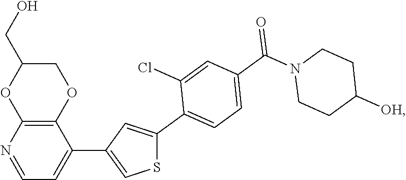 [SMILES: O=C(c1ccc(-c2cc(-c3ccnc4c3OCC(CO)O4)cs2)c(Cl)c1)N1CCC(O)CC1]

or a pharmaceutically acceptable salt, solvate, tautomer, isotope, or isomer of any of the foregoing.

Embodiment III-36. The compound of Embodiment III-1 or III-2, wherein the compound is:

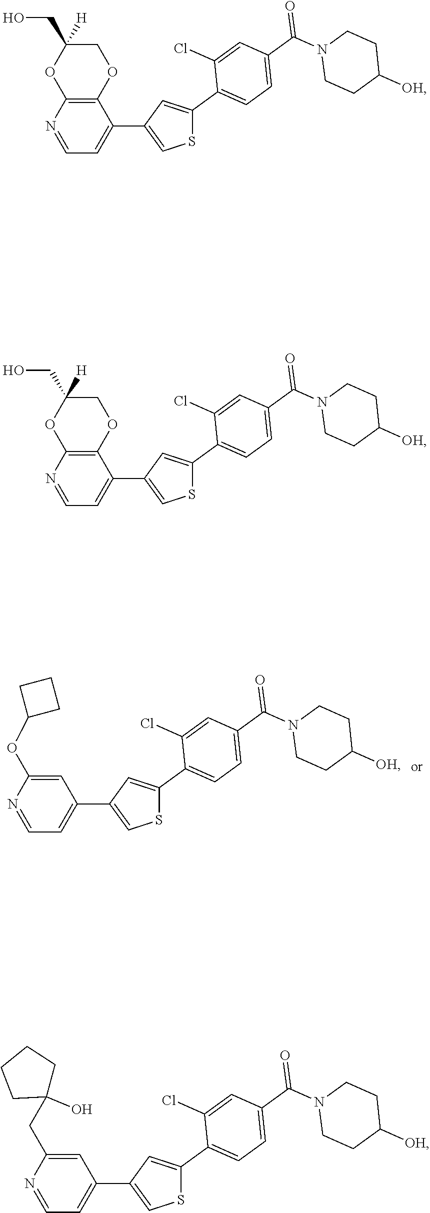 [SMILES: O=C(c1ccc(-c2cc(-c3ccnc4c3OC[C@H](CO)O4)cs2)c(Cl)c1)N1CCC(O)CC1 ; O=C(c1ccc(-c2cc(-c3ccnc4c3OC[C@@H](CO)O4)cs2)c(Cl)c1)N1CCC(O)CC1 ; O=C(c1ccc(-c2cc(-c3ccnc(OC4CCC4)c3)cs2)c(Cl)c1)N1CCC(O)CC1 ; O=C(c1ccc(-c2cc(-c3ccnc(CC4(O)CCCC4)c3)cs2)c(Cl)c1)N1CCC(O)CC1]

or a pharmaceutically acceptable salt, solvate, tautomer, or isotope, or isomer of any of the foregoing.

Embodiment III-37. The compound of Embodiment III-1, wherein the compound is:

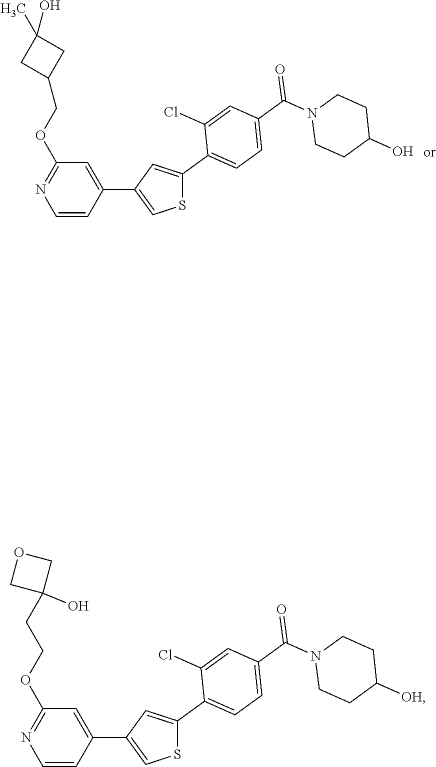 [SMILES: CC1(O)CC(COc2cc(-c3csc(-c4ccc(C(=O)N5CCC(O)CC5)cc4Cl)c3)ccn2)C1 ; O=C(c1ccc(-c2cc(-c3ccnc(OCCC4(O)COC4)c3)cs2)c(Cl)c1)N1CCC(O)CC1]

or a pharmaceutically acceptable salt, solvate, tautomer, isotope, or isomer of any of the foregoing.

Embodiment III-38. A pharmaceutical composition, comprising the compound of any one of Embodiments III-1 to III-37, or a pharmaceutically acceptable salt, solvate, tautomer, isotope, or isomer thereof, and a pharmaceutically acceptable excipient.

Embodiment III-39. A method of inhibiting a sterol regulatory element-binding protein (SREBP), comprising contacting the SREBP or contacting an SREBP cleavage activating-protein (SCAP) with an effective amount of a compound of any one of Embodiments III-1 to III-37, or a pharmaceutically acceptable salt, solvate, tautomer, isotope, or isomer thereof, or the pharmaceutical composition of Embodiment III-36.

Embodiment III-40. A method of inhibiting the proteolytic activation of a sterol regulatory element-binding protein (SREBP), comprising contacting an SREBP cleavage activating-protein (SCAP) with an effective amount of a compound of any one of Embodiments III-1 to III-37, or a pharmaceutically acceptable salt, solvate, tautomer, isotope, or isomer thereof, or the pharmaceutical composition of Embodiment III-38.

Embodiment III-41. A method of treating a disorder in a subject in need thereof, comprising administering to the subject in need thereof an effective amount of a compound of any one of Embodiments III-1 to III-37, or a pharmaceutically acceptable salt, solvate, tautomer, isotope, or isomer thereof, or the pharmaceutical composition of Embodiment III-38.

Embodiment III-42. A method of treating a disorder in a subject in need thereof, wherein the disorder is mediated by a sterol regulatory element-binding protein (SREBP), comprising administering to the subject in need thereof an effective amount of a compound of any one of Embodiments III-1 to III-37, or a pharmaceutically acceptable salt, solvate, tautomer, isotope, or isomer thereof, or the pharmaceutical composition of Embodiment III-38.

Embodiment III-43. A compound of any one of Embodiments III-1 to III-37, or a pharmaceutically acceptable salt, solvate, tautomer, isotope, or isomer thereof, for use in inhibiting a sterol regulatory element-binding protein (SREBP).

Embodiment III-44. A compound of any one of Embodiments III-1 to III-37, or a pharmaceutically acceptable salt, solvate, tautomer, isotope, or isomer thereof, for use in inhibiting the proteolytic activation of a sterol regulatory element-binding protein (SREBP).

Embodiment III-45. A compound of any one of Embodiments III-1 to III-37, or a pharmaceutically acceptable salt, solvate, tautomer, isotope, or isomer thereof, for use in treating a disorder in a subject in need thereof.

Embodiment III-46. A compound of any one of Embodiments III-1 to III-37, or a pharmaceutically acceptable salt, solvate, tautomer, isotope, or isomer thereof, for use in treating a disorder in a subject in need thereof, wherein the disorder is mediated by a sterol regulatory element-binding protein (SREBP).

Embodiment III-47. Use compound of any one of Embodiments III-1 to III-37, or a pharmaceutically acceptable salt, solvate, tautomer, isotope, or isomer thereof, in the manufacture of a medicament for inhibiting a sterol regulatory element-binding protein (SREBP).

Embodiment III-48. Use of a compound of any one of Embodiments III-1 to III-37, or a pharmaceutically acceptable salt, solvate, tautomer, isotope, or isomer thereof, in the manufacture of a medicament for inhibiting the proteolytic activation of a sterol regulatory element-binding protein (SREBP).

Embodiment III-49. Use of a compound of any one of Embodiments III-1 to III-37, or a pharmaceutically acceptable salt, solvate, tautomer, isotope, or isomer thereof, in the manufacture of a medicament for treating a disorder in a subject in need thereof.

Embodiment III-50. Use of a compound of any one of Embodiments III-1 to III-37, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

### EXAMPLES

The following Examples are merely illustrative and are not meant to limit any aspects of the present disclosure in any way.

### Synthesis Example 1: Preparation of reagent (3-chloro-4-(4-(4,4,5,5-tetramethyl-1,3,2-dioxaborolan-2-yl)thiophen-2-yl)phenyl)(4-hydroxypiperidin-1-yl)methanone

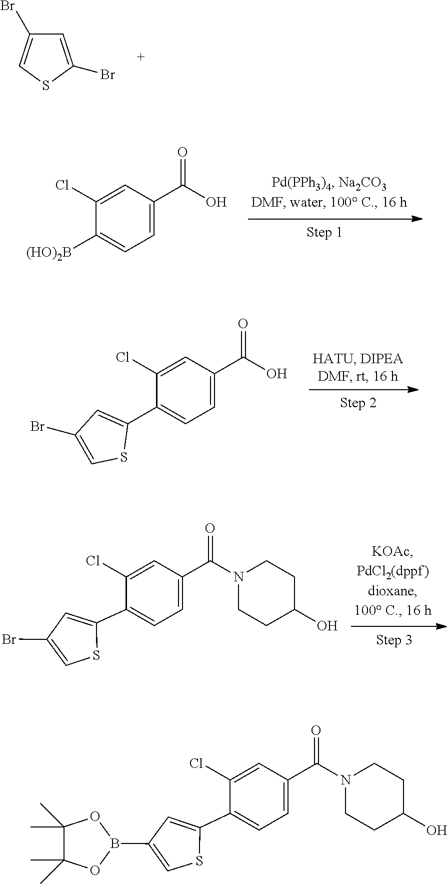 [SMILES: Brc1csc(Br)c1 ; O=C(O)c1ccc(B(O)O)c(Cl)c1 ; O=C(O)c1ccc(-c2cc(Br)cs2)c(Cl)c1 ; O=C(c1ccc(-c2cc(Br)cs2)c(Cl)c1)N1CCC(O)CC1 ; CC1(C)OB(c2csc(-c3ccc(C(=O)N4CCC(O)CC4)cc3Cl)c2)OC1(C)C]

Step 1: 4-(4-bromothiophen-2-yl)-3-chlorobenzoic acid. A mixture of 2,4-dibromothiophene (0.500 g, 2.066 mmol), 4-borono-3-chlorobenzoic acid (0.518 g, 2.583) and Na₂CO₃ (0.559 g, 5.371 mmol) in DMF and water in a glass seal tube was purged with nitrogen gas for 10 minutes. After adding palladium tetrakis (0.239 g, 0.206 mmol) the mixture was again purged with nitrogen gas for 10 minutes, sealed and then stirred at 100° C. for 16 h. The reaction mixture was then cooled to RT and evaporated under reduced pressure and 10 ml water was added. The precipitated solid was filtered, washed with cold water (5×2 mL) and finally dried under reduced pressure to give 4-(4-bromothiophen-2-yl)-3-chlorobenzoic acid (0.460 g, (70.76%) as an off white solid.

Step 2: (4-(4-bromothiophen-2-yl)-3-chlorophenyl)(4-hydroxypiperidin-1-yl)methanone. To a solution of 4-(4-bromothiophen-2-yl)-3-chlorobenzoic acid (0.450 g, 1.428 mmoles), in DMF (1.8 mL, 20 v), was added DIPEA (0.790 mL 4.284 mmoles) and HATU (0.815 g, 2.142 mmoles). After stirring for 30 min at RT, 4-hydroxy piperidine (0.173 g, 1.714 mmoles) was added and stirred was continued for another 16 h. The reaction mixture was diluted with ethyl acetate and washed with cold water (4×5 ml). The organic layer was separated, dried over anhydrous sodium sulfate and concentrated under reduced pressure to obtain crude material which was purified by column chromatography (silica gel: #230-400) using 20-60% EtOAc in hexane as eluent to afford (4-(4-bromothiophen-2-yl)-3-chlorophenyl)(4-hydroxypiperidin-1-yl)methanone (0.270 g, 50.0%) as a pale yellow semi-solid. ¹H NMR (400 MHz, DMSO d6): δ (ppm): 7.87 (1H, J=1.6 Hz, d), 7.72-7.70 (1H, J=8 Hz, d), 7.60-7.59 (1H, J=1.6 Hz, d), 7.52-7.51 (1H, J=1.6 Hz, d), 7.42-7.40 (1H, J=1.6 Hz, d), 4.80-4.79 (1H, J=1.6 Hz, d), 4.03-3.98 (1H, m), 3.77-3.71 (1H, m), 3.48 (1H, m), 3.29-3.25 (2H, m), 2.73 (1H, m), 1.98-1.90 (2H, m), 1.78-1.72 (2H, m), LCMS 82.45% (m/z=399.06) [M+H]).

Step 3: (3-chloro-4-(4-(4,4,5,5-tetramethyl-1,3,2-dioxaborolan-2-yl)thiophen-2-yl)phenyl)(4-hydroxypiperidin-1-yl)methanone. A mixture of (4-(4-bromothiophen-2-yl)-3-chlorophenyl)(4-hydroxypiperidin-1-yl)methanone (0.100 g, 0.249 mmol) as prepared in Step 5, bis(pinacolatodiborane) (0.126 g, 0.499 mmol) and potassium acetate (0.050 g, 0.499 mmol) in dioxan (2 mL, 20v) in a 5 mL glass seal tube was purged with nitrogen gas for 10 minutes. After adding Pd(dppf)Cl₂ (0.018 g, 0.025 mmol) it was again purged with nitrogen gas for 10 minutes, and the tube was sealed. The reaction mass was heated at 80° C. for 16 h and then cooled to RT and evaporated under reduced pressure. 5 ml water was added to the residue and the product was extracted into EtOAc (3×15 mL). The extracts were combined, washed with brine (3×10 mL) and dried over anhydrous sodium sulfate. The solution was concentrated under reduced pressure to give (3-chloro-4-(4-(4,4,5,5-tetramethyl-1,3,2-dioxaborolan-2-yl)thiophen-2-yl)phenyl)(4-hydroxypiperidin-1-yl)methanone (0.180 g, crude) as a black-brown semi-solid which was used as such for the next step.

### Synthesis Example 2: (3-chloro-4-(4-(2-(tetrahydro-2H-pyran-4-yl)pyridin-4-yl)thiophen-2-yl)phenyl)(4-hydroxypiperidin-1-yl)methanone (Compound I-1628752)

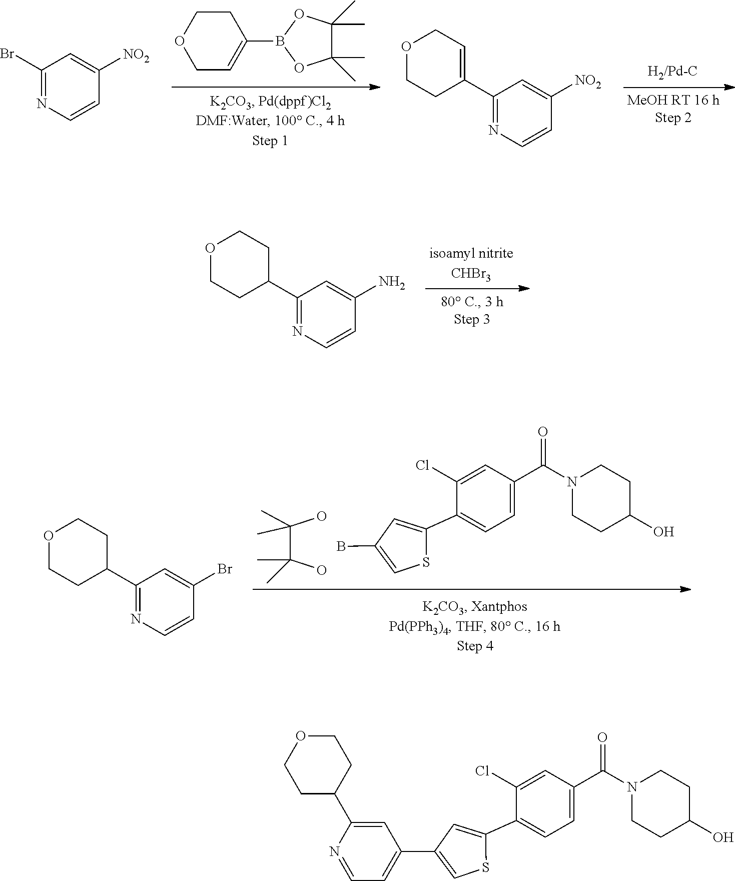

Step 1: 2-(3,6-dihydro-2H-pyran-4-yl)-4-nitropyridine. A mixture of 2-bromo-4-nitropyridine (0.400 g, 1.970 mmol), 2-(3,6-dihydro-2H-pyran-4-yl)-4,4,5,5-tetramethyl-1,3,2-dioxaborolane (0.496 g, 2.364 mmol) and K₂CO₃ (0.544 g, 3.940) in DMF:Water was purged with nitrogen gas for 10 minutes. After adding Pd(dppf)Cl₂ (0.144 g, 0.197 mmol) it was again purged with nitrogen gas for 10 minutes and the reaction mass was heated to 100° C. for 16 h. The reaction mixture was cooled to RT and evaporated to dryness under reduced pressure. 5 ml of water added, and the product was extracted into EtOAc (3×15 mL). The extracts were combined, washed with brine (3×10 mL) and dried over anhydrous sodium sulfate. The solution was concentrated under reduced pressure to get crude product which was purified by column chromatography with EtOAc in hexanes (0 to 25%) to afford 2-(3,6-dihydro-2H-pyran-4-yl)-4-nitropyridine (0.220 g, 54.87%) as an off white solid. ¹H NMR (400 MHz, CDCl₃): δ (ppm): 8.84 (1H, J=5.6 Hz, d), 8.08 (1H, J 1.6 Hz, d), 7.87-7.85 (1H, m), 6.92-6.90 (1H, m), 4.43-4.41 (2H, m), 3.99-3.96 (2H, m), 2.70-2.66 (2H, m), LCMS: 95.0% (m/z=207.1 [M+H]).

Step 2: 2-(tetrahydro-2H-pyran-4-yl)pyridin-4-amine. To a solution of 2-(3,6-dihydro-2H-pyran-4-yl)-4-nitropyridine (0.200 g, 0.9704 mmole) in MeOH was added Pd—C (10%) (0.100 g, 0.5 wt/wt 0.5 equiv) and kept under H₂ balloon pressure at RT for 16 h. The reaction mass was then filtered through a celite bed and the bed washed with methanol. The combined filtrates were concentrated to give 2-(tetrahydro-2H-pyran-4-yl)pyridin-4-amine (0.160 g, 93.6%) as a white solid. ¹H NMR (400 MHz, DMSO d6): δ (ppm): 7.89 (1H, d), 6.32-6.28 (2H, m), 5.86 (2H, s), 3.93-3.83 (2H, m), 3.42-3.36 (2H, m), 2.64-2.61 (1H, m), 1.68-1.63 (4H, m); LCMS 73.74% (m/z=179.14) [M+H]).

Step 3: 4-bromo-2-(tetrahydro-2H-pyran-4-yl)pyridine. To a solution of 2-(tetrahydro-2H-pyran-4-yl)pyridin-4-amine (0.160 g, 0.897 mmol) in CHBr₃ (4.8 mL) was slowly added isoamyl nitrite (2.4 mL) and the reaction mass was heated to 85° C. for 3 h. The reaction mass was then cooled to RT and quenched with 25 mL of aq. NaOH. Organic layer was separated, dried over sodium sulphate and evaporated to give a crude product which was purified by column chromatography (silica gel: #100-200) using 0-15% EtOAc in hexane as eluent to afford 4-bromo-2-(tetrahydro-2H-pyran-4-yl)pyridine (0.070 g, 32.40%) as yellow solid

Step 4: (3-chloro-4-(4-(2-(tetrahydro-2H-pyran-4-yl)pyridin-4-yl)thiophen-2-yl)phenyl)(4-hydroxypiperidin-1-yl)methanone. A mixture of 4-bromo-2-(tetrahydro-2H-pyran-4-yl)pyridine (0.060 g, 0.2478 mmol), (3-chloro-4-(4-(4,4,5,5-tetramethyl-1,3,2-dioxaborolan-2-yl)thiophen-2-yl)phenyl)(4-hydroxypiperidin-1-yl)methanone (0.150 g, 0.2478 mmol) as prepared in Example 1 and potassium carbonate (0.068 g, 0.4956 mmol) in THE (1.2 mL, 20v) was charged to a 5 mL glass seal tube and purged with nitrogen gas for 10 minutes. After adding xantphos (0.014 g, 0.0247 mmol) and palladium tetrakis (0.029 g, 0.0247 mmol) it was again purged with nitrogen gas for 10 minutes. The reaction mass was heated to 80° C. for 16 h, cooled to RT and evaporated under reduced pressure. 5 ml of water was added to the residue and the product was extracted into EtOAc (3×15 mL). The extracts were combined, washed with brine (3×10 mL) and dried over anhydrous sodium sulfate. The solution was concentrated under reduced pressure to give a crude product which was purified by Prep. HPLC to give (3-chloro-4-(4-(2-(tetrahydro-2H-pyran-4-yl)pyridin-4-yl)thiophen-2-yl)phenyl)(4-hydroxypiperidin-1-yl)methanone (0.026 g, 21.7%) as an off white solid. ¹H NMR (400 MHz, DMSO d6): δ (ppm): 8.53 (1H, J=5.2 Hz, d), 8.38 (1H, J=1.6 Hz, d), 8.10 (1H, J=1.6 Hz, d), 7.86-7.84 (1H, J=8.0 Hz, d), 7.71 (1H, s), 7.63-7.61 (2H, m), 7.46-7.43 (1H, J=1.6 Hz, dd), 4.80 (1H, s), 3.99-3.96 (3H, m), 3.77-3.74 (1H, m), 3.50-3.43 (3H, m), 3.20 (2H, m), 3.00-2.95 (1H, m), 1.91-1.79 (6H, m), 1.39 (2H, m). LCMS 99.64% (m/z=483. [M+H]), Purity by HPLC 98.76%.

### Synthesis Example 3: (3-chloro-4-(4-(2-(tetrahydrofuran-3-yl)pyridin-4-yl)thiophen-2-yl)phenyl)(4-hydroxypiperidin-1-yl)methanone (Compound I-1628763)

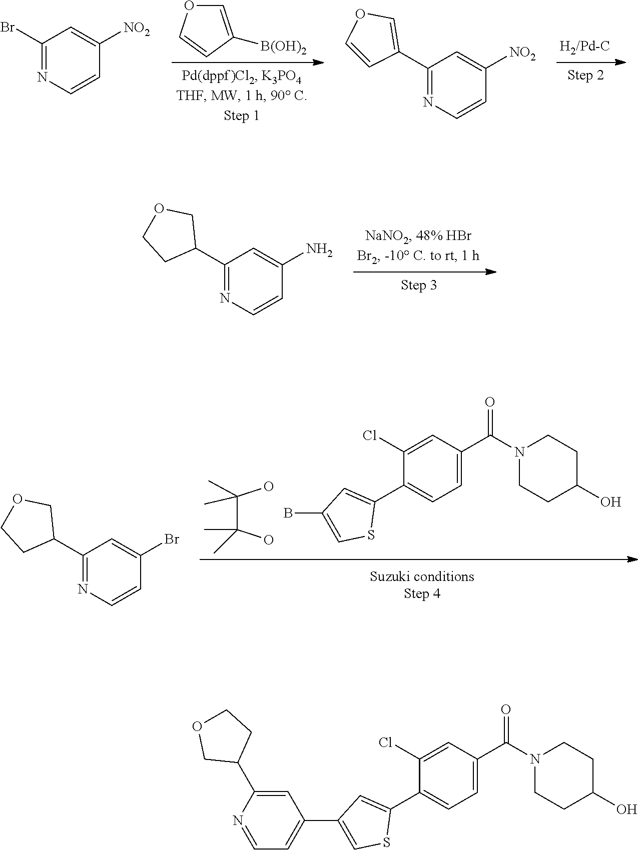

Step 1: 2-(furan-3-yl)-4-nitropyridine. To a stirred solution of 2-bromo-4-nitropyridine (0.35 g, 1.72 mmol) in THF (3.5 mL) was added furan-3-ylboronic acid (0.21 g, 1.89 mmol) followed by addition of K₃PO₄ (1.1 g, 5.17 mmol) at 25-30° C. The reaction mixture was purged with nitrogen for 15 minutes and then Pd(dppf)Cl2 (0.12 g, 0.17 mmol) was added and the reaction mixture again purged with nitrogen for 10 minutes. The reaction mixture was heated for 1 h at 90° C. in a sealed tube. The reaction mixture was then cooled to RT, water was added and the product extracted into ethyl acetate. The organic layer was washed with brine, dried over sodium sulfate, filtered and concentrated to give a crude product which was purified by silica gel column chromatography eluting with 0-30% ethyl acetate in pet ether to give 2-(furan-3-yl)-4-nitropyridine 0.25 g (78.0%) as a yellow solid. ¹H NMR (400 MHz, CDCl₃): δ 8.89-8.87 (m, 1H), 8.18-8.14 (m, 2H), 7.88-7.86 (m, 1H), 7.58-7.57 (m, 1H), 6.98-6.97 (m, 1H). LCMS: 93.40% (m/z=162.1 [M+H]).

Step 2: 2-(tetrahydrofuran-3-yl)pyridin-4-amine. To a stirred solution of 2-(furan-3-yl)-4-nitropyridine (0.25 g, 11.25 mmol, 1.0 eq) in methanol:EtOAc (2.5 mL: 2.5 mL) in a steel reaction vessel, was added Pd—C (0.075 g). The reaction vessel was filled with hydrogen (60 psi), sealed and kept at 50° C. for 16 h. The reaction mixture was cooled to RT, filtered through celite pad and the pad was washed with methanol. The filtrates were concentrated under reduced pressure to afford 2-(tetrahydrofuran-3-yl)pyridin-4-amine (0.20 g) as a gum which was used in the next without purification.

Step 3: 4-bromo-2-(tetrahydrofuran-3-yl)pyridine. Aq.HBr was added to 2-(tetrahydrofuran-3-yl)pyridin-4-amine (0.15 g, 0.91 mmol, 1.0 eq) at −15° C., and after 10 minutes of stirring, NaNO₂ (0.30 g, 4.57 mmol, 5.0 eq) in water (2.0 mL) was added. The reaction mixture was stirred at −15° C. for 1 h. Then Br₂ (0.28 g, 1.82 mmol, 2.0 eq) was added. After 1 hour at RT the reaction mixture was basified with NaHCO₃ soln and the product was extracted into EtOAc (3×15 mL). The extracts were combined, dried over anhydrous sodium sulfate and concentrated under reduced pressure to obtain crude product which was purified by column chromatography, eluting with 15% of EtOAc in pet ether to give 4-bromo-2-(tetrahydrofuran-3-yl)pyridine (0.110 g, %) as a light yellow gummy liquid. ¹H NMR (400 MHz, CDCl₃): δ 8.46 (s, 1H), 7.80-7.74 (m, 2H), 4.03-3.90 (m, 4H), 2.64 (d, J=8.0 Hz, 1H), 2.14 (d, J=8.0 Hz, 2H).

Step 4: (3-chloro-4-(4-(2-(tetrahydrofuran-3-yl)pyridin-4-yl)thiophen-2-yl)phenyl)(4-hydroxypiperidin-1-yl)methanone (I-1628763). To a stirred solution of 4-bromo-2-(tetrahydrofuran-3-yl)pyridine (0.102 g, 0.41 mmol, 1.0 eq) in Dioxane:Water (1.5 mL) in a glass tube, was added (3-chloro-4-(4-(4,4,5,5-tetramethyl-1,3,2-dioxaborolan-2-yl)thiophen-2-yl)phenyl)(4-hydroxypiperidin-1-yl)methanone (0.15 g, 0.45 mmol, 1.1 eq) and K₂CO₃ (0.154 g, 1.118 mmol, 2.5 eq) at room temperature under a nitrogen atmosphere. The reaction mass was purged for 15 minutes with nitrogen. Then palladium Tetrakis (0.048 g, 0.041 mmol, 0.1 eq) was added and the reaction mixture was again purged for 10 minutes with nitrogen. The reaction tube was sealed and stirred at 80° C. for 16 h after which the residue was diluted with water (10 mL) and the product was extracted into EtOAc (3×10 mL). The extracts were combined, dried over anhydrous sodium sulfate and concentrated under reduced pressure to obtained crude product was purified by Prep.HPLC to give (3-chloro-4-(4-(2-(tetrahydrofuran-3-yl)pyridin-4-yl)thiophen-2-yl)phenyl)(4-hydroxypiperidin-1-yl)methanone as a white solid. ¹H NMR (400 MHz, DMSO): δ 8.54 (d, J=5.2 Hz, 1H), 8.37 (d, J=1.6 Hz, 1H), 8.08 (d, J=1.6 Hz, 1H), 7.84 (d, J=8.0 Hz, 1H), 7.73 (d, J=1.2 Hz, 1H), 7.64 (m, 2H), 7.46 (m, 1H), 4.80 (s, 1H), 4.13-4.09 (m, 1H), 3.97-3.91 (m, 2H), 3.87-3.81 (m, 1H), 3.79-3.76 (m, 2H), 3.61-3.45 (m, 2H), 3.30-3.20 (m, 2H), 2.30-2.20 (m, 2H), 1.79-1.74 (m, 2H), 1.49-1.39 (m, 2H). LCMS: 98.29% (m/z=469.43 [M+H]+).

The following compounds were prepared in a similar fashion to Synthesis Example 3:

| | |
|---|---|
| 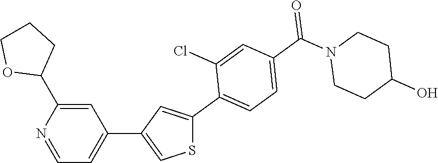 [SMILES: O=C(c1ccc(-c2cc(-c3ccnc(C4CCCO4)c3)cs2)c(Cl)c1)N1CCC(O)CC1] | (3-chloro-4-(4-(2-(tetrahydrofuran-2-yl)pyridin-4-yl)thiophen-2- yl)phenyl)(4-hydroxypiperidin-1-yl)methanone (Compound I-1628777) 1H NMR (400 MHz, DMSO): δ(ppm): 8.54 (d, J = 5.20 Hz, 1H), 8.38 (d, J = 1.60 Hz, 1H), 8.05 (d, J = 1.60 Hz, 1H), 7.85 (d, J = 8.00 Hz, 1H), 7.75 (d, J = 0.80 Hz, 1H), 7.67 (dd, J = 1.60, J = 5.20 Hz, 1H), 7.62 (d, J = 1.60 Hz, 1H), 7.44 (dd, J = 1.60, J = 7.80 Hz, 1H), 4.92-4.93 (m, 1H), 4.81 (d, J = 4.00 Hz, 1H), 4.04-4.06 (m, 2H), 3.86-3.87 (m, 1H), 3.73-3.74 (m, 1H), 3.55-3.48 (m, 1H), 3.25-3.12 (m, 2H), 2.31-2.32 (m, 1H), 1.93-1.94 (m, 3H), 1.74-1.75 (m, 2H), 1.45-1.35 (2H). LCMS: (469.33[M + H]) 99.57% |

### Synthesis Example 4: (4-(4-(2-(1,3-dioxolan-2-yl)pyridin-4-yl)thiophen-2-yl)-3-chlorophenyl)(4-hydroxypiperidin-1-yl)methanone (Compound I-1628743)

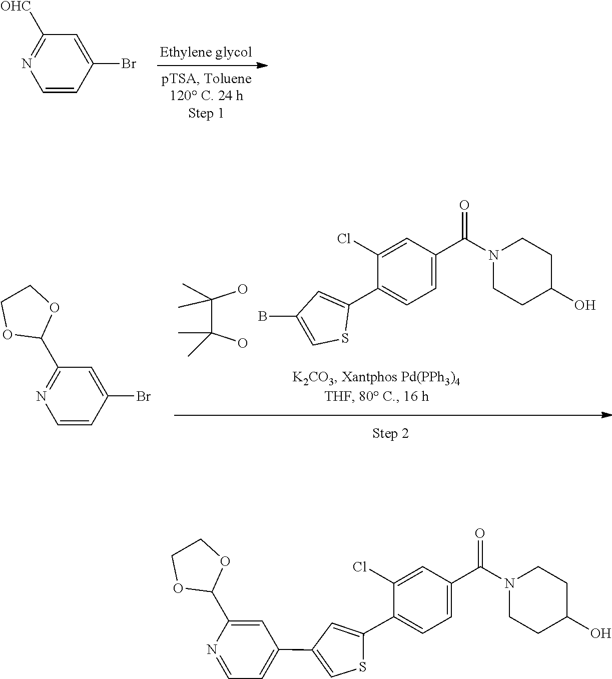

Step 1: 4-bromo-2-(1,3-dioxolan-2-yl)pyridine. A solution of 4-bromopinacolinaldehyde (0.200 g, 1.075 mmoles), ethylene glycol (1.330 g, 21.504 mmoles), and pTSA (0.014 g, 0.072 mmoles), in toluene was refluxed under nitrogen atmosphere for 24 h. The reaction mixture was cooled, washed with 10% Na₂CO₃ (15×3 mL) and water (15×3 mL). The organic layer was dried over sodium sulphate and concentrated to 4-bromo-2-(1,3-dioxolan-2-yl)pyridine (0.200 g, 80.87%) as pale yellow liquid. ¹H NMR (400 MHz, CDCl₃): δ (ppm): 8.43 (1H, J=5.2 Hz, d), 7.72 (1H, J 2 Hz, d), 7.47-7.45 (1H, m), 5.83 (1H, s), 4.18-4.06 (4H, m).

Step 2: (4-(4-(2-(1,3-dioxolan-2-yl)pyridin-4-yl)thiophen-2-yl)-3-chlorophenyl)(4-hydroxypiperidin-1-yl)methanone (I-1628743. A solution of 4-bromo-2-(1,3-dioxolan-2-yl)pyridine (0.077 g, 0.335 mmol), (3-chloro-4-(4-(4,4,5,5-tetramethyl-1,3,2-dioxaborolan-2-yl)thiophen-2-yl)phenyl)(4-hydroxypiperidin-1-yl)methanone (0.150 g, 335 mmol) and potassium carbonate (0.093 g, 0.670 mmol) in THF (3 mL, 20v) in a 5 mL glass seal tube was purged with nitrogen gas for 10 minutes. After adding xantphos (0.019 g, 0.033 mmol) and palladium tetrakis (0.033 g, 0.033 mmol) it was again purged with nitrogen gas for 10 minutes. The tube was sealed and was heated to 80° C. for 16 h. The reaction mixture was cooled to RT and evaporated to dryness under reduced pressure. 5 ml water was added to the residue and the product was extracted into EtOAc (3×15 mL). The extracts were combined, washed with brine (3×10 mL), dried over anhydrous sodium sulfate and concentrated under reduced pressure to give a crude product which was purified by Prep. HPLC to give (4-(4-(2-(1,3-dioxolan-2-yl)pyridin-4-yl)thiophen-2-yl)-3-chlorophenyl)(4-hydroxypiperidin-1-yl)methanone as an off white solid. ¹H NMR (400 MHz, DMSO d6): δ (ppm): 8.59-8.58 (1H, J=5.2 Hz, d), 8.43 (1H, J 1.6 Hz, d), 8.90 (1H, J=1.6 Hz, d), 7.86-7.81 (3H, m), 7.62-7.61 (1H, J=1.6 Hz, d), 7.7.45-7.43 (1H, J=1.6 Hz, dd), 5.78 (1H, s), 4.80 (1H, J=3.6 Hz, d), 4.19-4.16 (2H, m), 4.03-4.02 (2H, m), 3.77-3.76 (1H, m), 3.47-54 (1H, m), 3.22-3.21 (2H, m), 1.80-1.85 (2H, m), 1.39 (2H, m), 1.23 (1H, m). LCMS 99.13% (m/z=471.39 [M+H]), Purity by HPLC 99.55%.

### Synthesis Example 5: (3-chloro-4-(4-(2-(1-(hydroxymethyl)cyclopropyl)pyridin-4-yl)thiophen-2-yl)phenyl)(4-hydroxypiperidin-1-yl)methanone (Compound I-16289610)

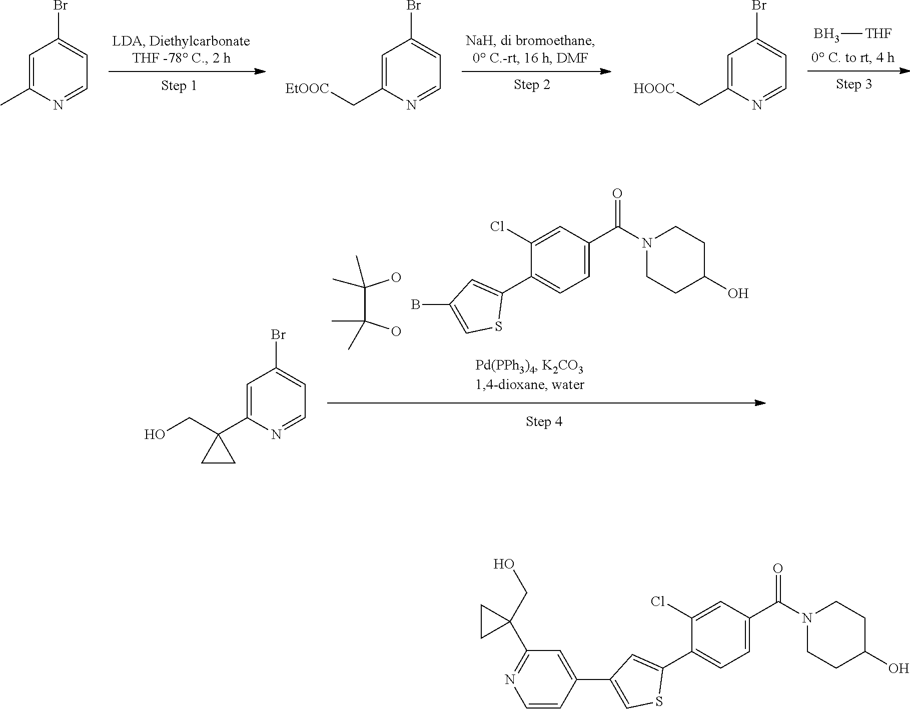

Step 1: ethyl 2-(4-bromopyridin-2-yl)acetate. To a stirred solution of di-isopropylamine (11.75 g) in THF (200 mL) was added n-BuLi (1.6 M in hexane) at −78° C. over 20 min. The temperature was raised to −15 to −25° C. for 1 h and then cooled to −50° C. when a solution of 4-bromo-2-methylpyridine (10 g, 58.13 mmol) in THF (25 mL) was added dropwise. The reaction mixture was stirred at −50° C. for 30 min, followed by addition of diethyl carbonate (10.3 g, 87.2 mmol). After stirring for another 2 hrs at −50° C. the reaction was quenched with saturated sodium chloride solution and extracted with EtOAc (2×250 mL). The organic layer was washed with brine (100 mL) and dried over anhydrous sodium sulfate. The solution was concentrated under reduced pressure, to give a crude compound, which was purified by column chromatography to give ethyl 2-(4-bromopyridin-2-yl)acetate (7.5 g, 53%) as a pale yellow liquid which was used directly in the next step.

Step 2: 1-(4-bromopyridin-2-yl)cyclopropane-1-carboxylic acid. To a stirred solution of ethyl 2-(4-bromopyridin-2-yl)acetate (7.50 g, 30.73 mmol) in DMF (100 mL) at 0° C. under nitrogen atmosphere, was added NaH (60%) (4.90 g, 123.4 mmol, 4 eq). The reaction mass was stirred at 0° C. for 15 mins followed by the addition of dibromoethane (26.18 g, 185.16 mmol, 6.0 eq) under nitrogen atmosphere. The reaction mixture was stirred at RT for 16 h and then quenched with ice water and extracted with EtOAc (3×50 mL). The aqueous layer was acidified up to pH 4 and extracted with DCM (3×100 mL). The DCM layer was dried over anhydrous sodium sulfate and concentrated under reduced pressure to give crude 1-(4-bromopyridin-2-yl)cyclopropane-1-carboxylic acid which was used as such in the next step.

Step 3: (1-(4-bromopyridin-2-yl)cyclopropyl)methanol. To a stirred solution of the product of Step 6 (4.0 g, 16.52 mmol, 1 eq) in THE (100 mL) under nitrogen atmosphere, at 0° C., was added BH₃-THF (82.56 mL, 82.62 mmol) dropwise. The mixture was stirred at room temperature for 4 h and then quenched with methanol (10 mL). The solvent was evaporated under reduced pressure and purified by column chromatography using 7% methanol in DCM to give (1-(4-bromopyridin-2-yl)cyclopropyl)methanol (2 g, 53%) as a pale yellow liquid. ¹H NMR (400 MHz, DMSO-d6): δ 8.30 (dd, J=0.4 Hz, 5.20 Hz, 1H), 7.73-(d, J=1.6 Hz, 1H), 7.42-7.40 (dd, J=2.00 Hz and 5.60 Hz, 1H), 4.85 (t, J=5.2 Hz, 1H), 3.72 (d, J=5.20 Hz, 2H), 1.24-1.11 (m, 2H), 0.93-0.90 (m, 2H).

Step 4: (3-chloro-4-(4-(2-(1-(hydroxymethyl)cyclopropyl)pyridin-4-yl)thiophen-2-yl)phenyl)(4-hydroxypiperidin-1-yl)methanone (I-16289610). To a solution of (1-(4-bromopyridin-2-yl)cyclopropyl)methanol (80 mg, 0.350 mmol, 1 eq) and (3-chloro-4-(4-(4,4,5,5-tetramethyl-1,3,2-dioxaborolan-2-yl)thiophen-2-yl)phenyl)(4-hydroxypiperidin-1-yl)methanone (257 mg, 0.704 mmol) in 1,4-dioxane:water (5 mL:1 mL) in a glass tube was added K₂CO₃ (145 mg, 1.05 mmol) and was purged with nitrogen gas for ten minutes. Tetrakis palladium (40 mg, 0.03 mmol) was added and the reaction mixture was again degassed under nitrogen atmosphere for 5 min. The glass tube was sealed and stirred at 80° C. for 16 h. The mixture was filtered through a celite bed and washed with ethyl acetate. The filtrate and washings were combined and concentrated under reduced pressure to afford crude product, which was purified by prep. HPLC to give (3-chloro-4-(4-(2-(1-(hydroxymethyl)cyclopropyl)pyridin-4-yl)thiophen-2-yl)phenyl)(4-hydroxypiperidin-1-yl)methanone (22 mg) as an off white solid. ¹H NMR (400 MHz, DMSO-d6): δ 8.46 (d, J=5.20 Hz, 1H), 8.33 (d, J=1.60 Hz, 1H), 8.04 (d, J=1.60 Hz, 1H), 7.84 (d, J=8.00 Hz, 1H), 7.77 (d, J=0.80 Hz, 1H), 7.62 (d, J=1.60 Hz, 1H), 7.54-7.51 (dd, J=1.6 Hz and 5.2 Hz, 1H), 7.46-7.43 (d, J=1.6 Hz and 8.0 Hz, 1H), 4.80 (t, J=6.00 Hz, 2H), 4.05-3.95 (br s, 1H), 3.83 (d, J=5.60 Hz, 2H), 3.80-3.70 (m, 1H), 3.55-3.45 (m, 1H), 3.25-3.15 (m, 2H), 1.85-1.65 (m, 2H), 1.45-1.30 (m, 2H), 1.16-1.13 (m, 2H), 0.93-0.91 (m, 2H).

The following compounds were prepared in a similar fashion to Synthesis Example 1 and Example 5:

| | |
|---|---|
| 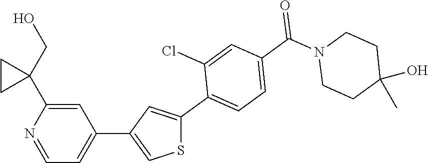 [SMILES: CC1(O)CCN(C(=O)c2ccc(-c3cc(-c4ccnc(C5(CO)CC5)c4)cs3)c(Cl)c2)CC1] | (3-chloro-4-(4-(2-(l-(hydroxymethyl)cyclopropyl)pyridin-4- yl)thiophen-2-yl)phenyl)(4-hydroxy-4-methylpiperidin-1- yl)methanone (Compound I-1629069) ¹H NMR (400 MHz, DMSO): δ 8.46 (d, J = 5.2 Hz, 1H), 8.33 (d, J = 1.6 Hz, 1H), 8.03 (d, J = 1.6 Hz, 1H), 7.83 (d, J = 7.6 Hz, 1H), 7.77 (d, J = 0.8 Hz, 1H), 7.62 (d, J = 1.6 Hz, 1H), 7.53 (dd, J = 1.6 Hz and 5.2 Hz, 1H), 7.45 (dd, J = 1.6 Hz and 8.0 Hz, 1H), 4.80 (t, J = 5.6 Hz, 1H), 4.44 (s, 1H), 4.08 (br s, 1H), 3.83 (d, J = 5.6 Hz, 2H), 3.34 (m, 1H), 3.22 (m, 2H), 1.52 (m, 4H), 1.16 (m, 5H), 0.92 (m, 2H). LCMS: 99.76% (483.32[M + H] ion present) |
| 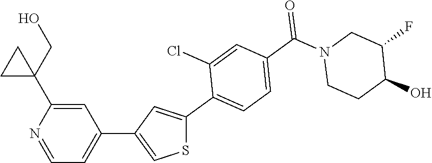 [SMILES: O=C(c1ccc(-c2cc(-c3ccnc(C4(CO)CC4)c3)cs2)c(Cl)c1)N1CC[C@H](O)[C@@H](F)C1] | (3-chloro-4-(4-(2-(l-(hydroxymethyl)cyclopropyl)pyridin-4- yl)thiophen-2-yl)phenyl)((3S,4S)-3-fluoro-4- hydroxypiperidin-1-yl)methanone (Compound I-1629121) 1H NMR (400 MHz, DMSO) δ 8.46 (d, J = 5.2 Hz, 1H), 8.34 (d, J = 1.2 Hz, 1H), 8.05 (d, J = 1.2 Hz, 1H), 7.86 (d, J = 8.0 Hz, 1H), 7.77 (s, 1H), 7.62 (d, J = 1.2 Hz, 1H), 7.53 (q, J = 2.4 Hz, 1H), 7.46 (q, J = 3.2 Hz, 1H), 5.43 (d, J = 3.6 Hz, 1H), 4.81 (t, J = 5.6 Hz, 1H), 4.48-4.36 (m, 1H), 3.84 (d, J = 5.6 Hz, 4H), 3.66-3.49 (m, 3H), 1.85 (s, 1H), 1.61-1.50 (m, 1H), 1.16-1.14 (m, 2H), 0.095-0.93 (m, 2H). LCMS: 99.65% (m/z = 487.0 [M + H]) ⁺ |
| 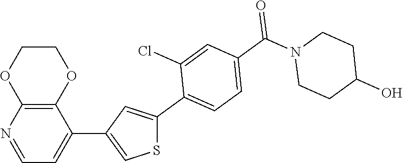 [SMILES: O=C(c1ccc(-c2cc(-c3ccnc4c3OCCO4)cs2)c(Cl)c1)N1CCC(O)CC1] | (3-chloro-4-(4-(2,3-dihydro-[1,4]dioxino[2,3-b]pyridin-8- yl)thiophen-2-yl)phenyl)(4-hydroxypiperidin-1-yl)methanone (I-1629066) ¹H NMR (400 MHz, DMSO): δ 8.23 (d, J = 1.6 Hz, 1H), 7.96 (d, J = 1.6 Hz, 1H), 7.78 (m, 2H), 7.61 (d, J = 1.6 Hz, 1H), 7.43 (dd, J = 1.6 Hz and 8.0 Hz, 1H), 7.32 (d, J = 5.2 Hz, 1H), 4.80 (d, J = 4.0 Hz, 1H), 4.47 (m, 2H), 4.36 (m, 2H), 3.99 (br s, 1H), 3.76 (m, 1H), 3.51 (br s, 1H), 3.21 (m, 2H), 1.76 (m, 2H), 1.39 (m, 2H). LCMS: 99.8 % (m/z = 457.35 [M + H]) |
| 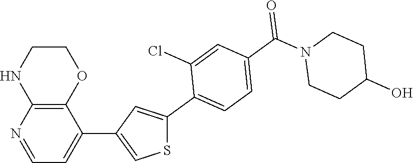 [SMILES: O=C(c1ccc(-c2cc(-c3ccnc4c3OCCN4)cs2)c(Cl)c1)N1CCC(O)CC1] | (3-chloro-4-(4-(3,4-dihydro-2H-pyrido[3,2-b][1,4]oxazin-8- yl)thiophen-2-yl)phenyl)(4-hydroxypiperidin-1-yl)methanone (I-1629067) ¹H NMR (400 MHz, DMSO) δ 8.13 (d, J = 1.2 Hz, 1H), 7.90 (d, J = 1.6 Hz, 1H), 7.77 (d, J = 8.0 Hz, 1H), 7.59 (m, 2H), 7.42(dd, J = 1.6 Hz and 8.0 Hz, 1H), 6.82 (d, J = 5.6 Hz, 1H), 6.78 (br s, 1H), 4.80 (d, J = 4.0 Hz, 1H), 4.21 (t, J = 4.4 Hz, 2H), 3.99 (br s, 1H), 3.75 (m, 1H), 3.46 (m, 3H), 3.21 (m, 2H), 1.77 (m, 2H), 1.38 (s, 2H). LCMS: 98.35 % (M + H = 423.2) |
| 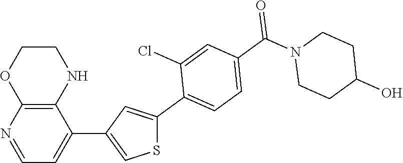 [SMILES: O=C(c1ccc(-c2cc(-c3ccnc4c3NCCO4)cs2)c(Cl)c1)N1CCC(O)CC1] | (3-chloro-4-(4-(2,3-dihydro-1H-pyrido[2,3-b][l,4]oxazin-8- yl)thiophen-2-yl)phenyl)(4-hydroxypiperidin-1-yl)methanone (I-1629084) ¹H NMR (400 MHz, DMSO) δ 7.95 (d, J = 1.5 Hz, 1H), 7.82 (d, J = 8.0 Hz, 1H), 7.74 (d, J = 1.5 Hz, 1H), 7.61 (d, J = 1.6 Hz, 1H), 7.44 (m, J = 3.2 Hz, 2H), 6.92 (d, J = 5.1 Hz, 1H), 5.67 (s, 1H), 4.80 (d, J = 3.9 Hz, 1H), 4.29 (t, J = 4.3 Hz, 2H), 3.99 (s, 1H), 3.75 (m, J = 4.0 Hz, 1H), 3.51 (s, 1H), 3.29 (m, J = 1.4 Hz, 4H), 1.77 (br s, 2H), 1.38 (br s, 2H). LCMS: 99.8 % (m/z = 457.35 [M + H]). |
| 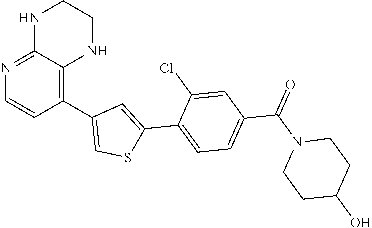 [SMILES: O=C(c1ccc(-c2cc(-c3ccnc4c3NCCN4)cs2)c(Cl)c1)N1CCC(O)CC1] | (3-chloro-4-(4-(l,2,3,4-tetrahydropyrido[2,3-b]pyrazin-8- yl)thiophen-2-yl)phenyl)(4-hydroxypiperidin-1- yl)methanone (I-1629068) ¹H NMR (400 MHz, DMSO): δ 7.83 (d, J = 1.6 Hz, 1H), 7.81 (d, J = 8.0 Hz, 1H), 7.67 (d, J = 1.2 Hz, 1H), 7.60 (d, J = 1.6 Hz, 1H), 7.42 (dd, J = 1.6 Hz and 8.0 Hz, 1H), 7.31 (d, J = 5.2 Hz, 1H), 6.47 (d, J = 5.2 Hz, 1H), 6.31(s, 1H), 5.23 (s, 1H), 4.80 (d, J = 4.0 Hz, 1H), 3.98 (br s, 1H), 3.75 (m, 1H), 3.51 (br s, 1H), 3.35 (br s, 2H), 3.21 (m, 4H), 1.76 (m, 2H), 1.39 (m, 2H). LCMS: 99.78% ((M + H) + = 455.29. |

### Synthesis Example 6: (3-chloro-4-(4-(2-(1-hydroxycyclobutyl)pyridin-4-yl)thiophen-2-yl)phenyl)(4-hydroxypiperidin-1-yl)methanone (Compound I-1629076)

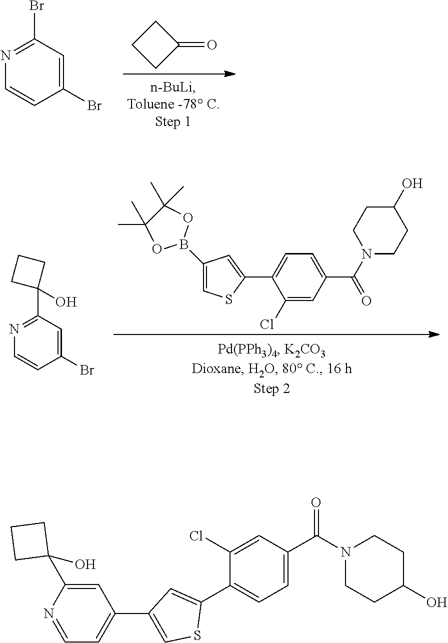 [SMILES: Brc1ccnc(Br)c1 ; O=C1CCC1 ; OC1(c2cc(Br)ccn2)CCC1 ; CC1(C)OB(c2csc(-c3ccc(C(=O)N4CCC(O)CC4)cc3Cl)c2)OC1(C)C ; O=C(c1ccc(-c2cc(-c3ccnc(C4(O)CCC4)c3)cs2)c(Cl)c1)N1CCC(O)CC1]

Step 1: 1-(4-bromopyridin-2-yl)cyclobutan-1-ol. To a stirred solution of 2,4-dibromopyridine (1.0 gm, 4.22 mmol) in Toluene (15 mL), was added n-BuLi (0.27 gm, 4.22 mmol) dropwise at −78° C. After 15 min, cyclobutanone in toluene (2 mL) was added dropwise at the same temperature. The reaction mass was stirred at −78° C. for 2 h and then quenched with (20%) NH₄Cl solution and the product was extracted into ethyl acetate (3×50 mL). The combined organic extracts were washed with brine solution (50 mL×2), dried over anhydrous sodium sulfate and concentrated under reduced pressure to give a crude product which was purified by silica gel column chromatography to give 1-(4-bromopyridin-2-yl)cyclobutan-1-ol (0.600 g, 63%) as a light yellow liquid. ¹H NMR 400 MHz, DMSO-d6: δ 8.35 (d, J=5.20 Hz, 1H), 7.75 (d, J=1.20 Hz, 1H), 7.38 (dd, J=1.60, 5.20 Hz, 1H), 4.73 (s, 1H), 2.49-2.51 (m, 4H), 2.10-2.00 (m, 1H), 1.89-1.85 (m, 1H). LCMS: 92.41% (228.08[M+H]⁺ ion present

Step 2: (3-chloro-4-(4-(2-(I-hydroxycyclobutyl)pyridin-4-yl)thiophen-2-yl)phenyl)(4-hydroxypiperidin-1-yl)methanone (I-1629076). To a stirred solution of 1-(4-bromopyridin-2-yl)cyclobutan-1-ol (0.2 g, 0.87 mmol) and (3-chloro-4-(4-(4,4,5,5-tetramethyl-1,3,2-dioxaborolan-2-yl)thiophen-2-yl)phenyl)(4-hydroxypiperidin-1-yl)methanone (0.58 g, 1.3 mmol) in dioxane:Water in a glass tube, was added K₂CO₃ (0.36 g, 2.62 mmol) at room temperature under nitrogen atmosphere. The reaction mass was purged for 5 min with nitrogen, then Pd(PPh₃)₄ (0.50 g, 0.043 mmol) was added and the reaction mixture was again purged for 10 min with nitrogen. The reaction tube was sealed and stirred at 80° C. for 16 h. The reaction mixture was filtered through celite bed and the celite bed was washed with EtOAc. The combined filtrates were washed with water, dried over anhydrous sodium sulfate and concentrated under reduced pressure to obtain crude material which was purified by reverse column chromatography using a C₁₈ column to give (3-chloro-4-(4-(2-(1-hydroxycyclobutyl)pyridin-4-yl)thiophen-2-yl)phenyl)(4-hydroxypiperidin-1-yl)methanone as an off as white solid (24 mg, 7%). 1H NMR (400 MHz, DMSO): δ 8.59 (dd, J=0.8 Hz and 5.6 Hz, 1H), 8.35 (d, J=1.2 Hz, 1H), 8.03 (d, J=1.2 Hz, 1H), 7.91 (s, 1H), 7.85 (d, J=8.0 Hz, 1H), 7.66 (m, 1H), 7.62 (d, J=1.6 Hz, 1H), 7.44 (dd, J=2.0 Hz and 8.0 Hz, 1H), 5.80 (br s, 1H), 4.81 (br s, 1H), 3.99 (br s, 1H), 3.76 (m, 1H), 3.50 (br s, 1H), 3.19 (br s, 2H), 2.61 (m, 2H), 2.25 (m, 2H), 2.0-1.70 (m, 4H), 1.39 (br s, 2H). LCMS: 99.6% (m/z=469.29 [M+H]).

The following compounds were prepared in a similar fashion to Synthesis Example 6:

| | |
|---|---|
| 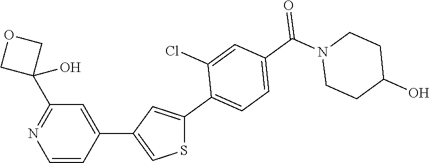 [SMILES: O=C(c1ccc(-c2cc(-c3ccnc(C4(O)COC4)c3)cs2)c(Cl)c1)N1CCC(O)CC1] | (3-chloro-4-(4-(2-(3-hydroxyoxetan-3-yl)pyridin-4-yl)thiophen-2- yl)phenyl)(4-hydroxypiperidin-1-yl)methanone (Compound I- 1629083) ¹H NMR (400 MHz, DMSO): δ 8.67 (q, J = 1.9 Hz, 1H), 8.38 (d, J = 1.5 Hz, 1H), 8.06 (d, J = 1.5 Hz, 1H), 7.93 (d, J = 1.0 Hz, 1H), 7.85 (d, J = 8.0 Hz, 1H), 7.74 (q, J = 2.3 Hz, 1H), 7.62 (d, J = 1.6 Hz, 1H), 7.44 (q, J = 3.2 Hz, 1H), 6.58 (s, 1H), 4.98 (d, J = 6.2 Hz, 2H), 4.81 (d, J = 3.9 Hz, 1H), 4.69 (d, J = 6.1 Hz, 2H), 3.98 (s, 1H), 3.76 (m, J = 3.9 Hz, 1H), 3.51 (s, 1H), 3.17 (brs, 2H), 1.79 (br s, 2H), 1.38 (br s, 2H). LCMS: 98.23 % (m/z = 471.22 [M + H]) |

### Synthesis Example 7: (3-chloro-4-(4-(2-(1-(hydroxymethyl)cyclobutyl)pyridin-4-yl)thiophen-2-yl)phenyl)(4-hydroxypiperidin-1-yl)methanone (Compound I-1629089)

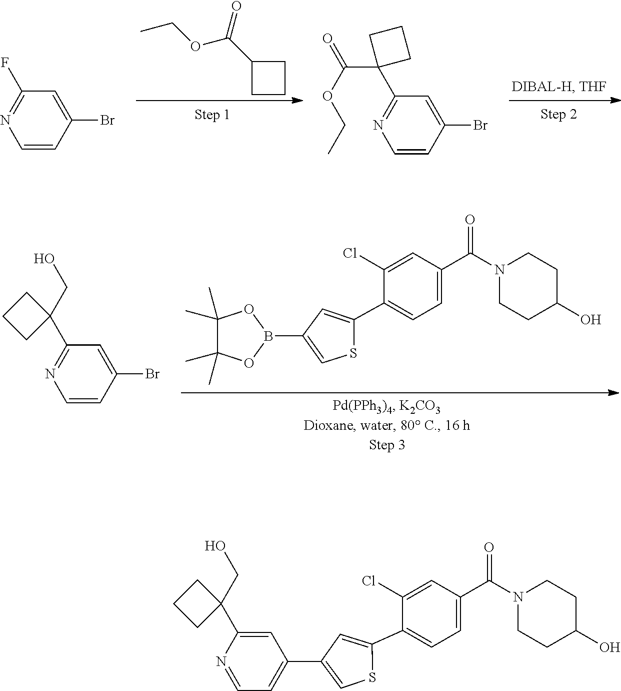

Step 1: ethyl 1-(4-bromopyridin-2-yl)cyclobutane-1-carboxylate. To a stirred solution of 2-fluoro-4-bromopyridine (1.g, 5.06 mmol) and ethyl cyclobutanecarboxylate (0.666 g, 5.84 mmol) in toluene was added LiHMDS (1.M soln. 11.6 mL, 11.69 mmol) at 0° C. The reaction mixture was stirred at 0° C. for 15 min and then at rt for 4 h. The reaction was quenched with aqueous NH₄Cl solution and the product was extracted into ethyl acetate (2×50 mL). The extracts were combined, dried over anhydrous sodium sulfate and concentrated under reduced pressure to obtain crude product which was purified by silica gel column chromatography to give ethyl 1-(4-bromopyridin-2-yl)cyclobutane-1-carboxylate as a brown gum. ¹H NMR 400 MHz, CDCl₃: δ 8.41 (dd, J=5.2 Hz and 0.4 Hz, 1H), 7.49 (dd, J=1.6 Hz and 0.4 Hz, 1H), 7.36 (dd, J=1.6 Hz and 5.20 Hz, 1H), 4.18-4.16 (m, 2H), 2.81-2.80 (m, 2H), 2.65-2.64 (m, 2H), 2.11 (m, 1H), 1.94-1.93 (m, 1H), 1.23 (t, J=7.20 Hz, 3H).

Step 2: (1-(4-bromopyridin-2-yl)cyclobutyl)methanol. To a stirred solution of ethyl 1-(4-bromopyridin-2-yl)cyclobutane-1-carboxylate (0.2 gm, 0.74 mmol) in THE (5 mL), was added DIBAL-H (0.315 gm, 2.22 mmol) dropwise at −78° C. The reaction mass was stirred at rt for 4 h. The reaction mixture was quenched by adding aq. 20% NH₄Cl solution and extracted with ethyl acetate (3×50 mL) and washed the organic layer with brine solution (50 mL×2). The organic layer was dried over anhydrous sodium sulfate and concentrated under reduced pressure to afford crude (1-(4-bromopyridin-2-yl)cyclobutyl)methanol (0.150 g) which was used for next step as such without any further purification.

Step 3: (3-chloro-4-(4-(2-(1-(hydroxymethyl)cyclobutyl)pyridin-4-yl)thiophen-2-yl)phenyl)(4-hydroxypiperidin-1-yl)methanone (I-1629089). To a stirred solution of the product of Step 2 (0.15 g, 0.618 mmol) and (3-chloro-4-(4-(4,4,5,5-tetramethyl-1,3,2-dioxaborolan-2-yl)thiophen-2-yl)phenyl)(4-hydroxypiperidin-1-yl)methanone (0.411 g, 0.92 mmol) in dioxane:water in a sealed tube, was added K₂CO₃ (0.255 g, 1.854 mmol) at room temperature under nitrogen atmosphere. The reaction mixture was purged for 5 min with nitrogen, then added Pd(PPh₃)4 (0.50 g, 0.043 mmol) and again purged for 10 min with nitrogen. The reaction tube was sealed and stirred at 80° C. for 16 h. The reaction mixture was filtered through a celite bed and the celite bed was washed with EtOAc. The combined filtrates were washed with water, dried over anhydrous sodium sulfate and concentrated under reduced pressure to obtain crude material which was purified by Prep HPLC to give (3-chloro-4-(4-(2-(1-(hydroxymethyl)cyclobutyl)pyridin-4-yl)thiophen-2-yl)phenyl)(4-hydroxypiperidin-1-yl)methanone as a white solid. ¹H NMR (400 MHz, DMSO): δ 8.53 (d, J=5.6 Hz, 1H), 8.34 (d, J=1.6 Hz, 1H), 8.05 (d, J=1.2 Hz, 1H), 7.85 (d, J=8.0 Hz, 1H), 7.61 (d, J=1.6 Hz, 1H), 7.57 (m, 2H), 7.44 (dd, J=2.0 Hz and 8.0 Hz, 1H), 4.78 (m, 2H), 3.99 (br s, 1H), 3.76 (m, 1H), 3.69 (s, 2H), 3.51 (br s, 1H), 3.17 (m, 2H), 2.40 (m, 2H), 2.21 (m, 2H), 1.97 (m, 1H), 1.78 (m, 3H), 1.40 (br s, 2H). LCMS: 97% (m/z=483.32 [M+H]).

### Synthesis Example 8: (3-chloro-4-(4-(2-cyclopropoxypyridin-4-yl)thiophen-2-yl)phenyl)(4-hydroxypiperidin-1-yl)methanone (Compound I-1629059)

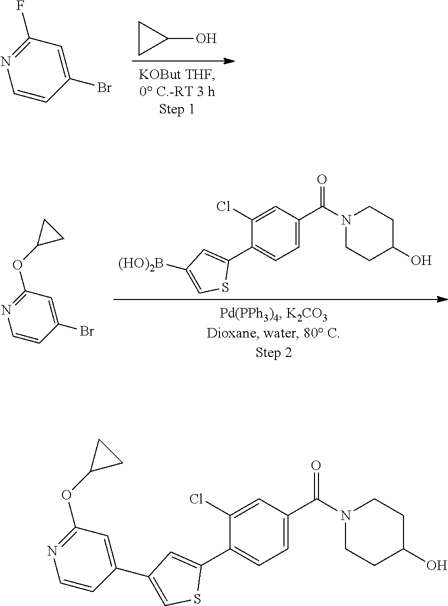

Step 1: 4-bromo-2-cyclopropoxypyridine. To a stirred solution of cyclpropanol (600 mg, 11.36 mmol) in THF (10.0 mL) at 0-5° C. was added KOBu^{-t}(1.99 g, 17.04 mmol) and the reaction mixture was stirred for 30 mins at 0-5° C. 2-fluoro-4-bromopyridine (1.0 g, 5.68 mmol) was dissolved in THF (5 mL) and added dropwise at 0-5° C. for about 10 mins and the reaction mixture was stirred for 3 h at rt. The reaction mixture was quenched with ice water and the product was extracted into ethyl acetate (3×50 mL). The combined organic layers were dried over anhydrous sodium sulfate and concentrated under reduced pressure to afford crude material which was purified by silica gel column chromatography to give 4-bromo-2-cyclopropoxypyridine (0.8 g, 66%) as a light yellow liquid. 1H NMR 400 MHz, CDCl₃-d₆: δ 8.06 (d, J=5.60 Hz, 1H), 7.07 (dd, J=1.60 Hz, 5.20 Hz, 1H), 6.98 (d, J=1.60 Hz, 1H), 4.15-4.13 (m, 1H), 0.82-0.80 (m, 4H).

Step 2: (3-chloro-4-(4-(2-cyclopropoxypyridin-4-yl)thiophen-2-yl)phenyl)(4-hydroxypiperidin-1-yl)methanone (I-1629059). To a solution of 4-bromo-2-cyclopropoxypyridine (150 mg, 0.93 mmol, 1 eq) and (5-(2-chloro-4-(4-hydroxypiperidine-1-carbonyl)phenyl)thiophen-3-yl)boronic acid (514 mg, 1.40 mmol) in 1,4-dioxane:water (7 mL:3 mL) was added K₂CO₃ (388 mg, 2.81 mmol). The reaction mixture was purged with nitrogen gas for 5 minutes, tetrakis palladium (108 mg, 0.09 mmol) was added and again purged with nitrogen. The reaction mixture was stirred at 80° C. for 16 h, cooled to RT and filtered through a celite bed which was then washed with ethyl acetate. The combined filtrates were evaporated under reduced pressure to afford crude to give a crude product which was purified by Prep-HPLC to give (3-chloro-4-(4-(2-cyclopropoxypyridin-4-yl)thiophen-2-yl)phenyl)(4-hydroxypiperidin-1-yl)methanone (75 mg, 18%) as an off white fluffy solid. 11H NMR 400 MHz, DMSO-d6: δ 8.37 (d, J=1.20 Hz, 1H), 8.23 (d, J=5.20 Hz, 1H), 8.05 (d, J=1.60 Hz, 1H), 7.83 (d, J=8.00 Hz, 1H), 7.61 (d, J=1.60 Hz, 1H), 7.45-7.42 (m, 2H), 7.25 (d, J=0.80 Hz, 1H), 4.80 (s, 1H), 4.29-4.25 (m, 1H), 4.05-3.9 (br s, 1H), 3.80-3.70 (i, 1H), 3.55-3.45 (br s, 1H), 3.30-3.10 (m, 2H), 1.75-1.64 (i, 2H), 1.45-1.30 (m, 2H), 0.79-0.69 (in, 4H). LCMS: 99.83 (m/z=455.38 [M+H])

The following compounds were prepared in a similar fashion to Synthesis Example 8:

| | |
|---|---|
| 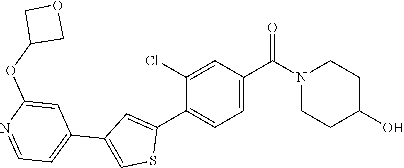 [SMILES: O=C(c1ccc(-c2cc(-c3ccnc(OC4COC4)c3)cs2)c(Cl)c1)N1CCC(O)CC1] | (3-chloro-4-(4-(2-(oxetan-3-yloxy)pyridin-4-yl)thiophen-2- yl)phenyl)(4-hydroxypiperidin-1-yl)methanone (Compound I- 1629100) ¹H NMR (400 MHz, DMSO): δ 8.40 (d, J = 1.6 Hz, 1H), 8.15 (d, J = 5.6 Hz, 1H), 8.09 (d, J = 1.6 Hz, 1H), 7.84 (d, J = 8.0 Hz, 1H), 7.61 (d, J = 1.6 Hz, 1H), 7.44 (m, 2H), 7.34 (d, J = 0.8 Hz, 1H), 5.65-5.55 (m, 1H), 4.91 (t, J = 7.2 Hz, 2H), 4.80 (d, J = 4.0 Hz, 1H), 4.59 (m, 2H), 3.99 (br s, 1H), 3.76 (m, 1H), 3.51 (br s, 1H), 3.23 (br s, 2H), 1.76 (br s, 2H), 1.39 (br s, 2H). LCMS: 98.49 % (m/z = 471.26 [M + H])⁺ |
| 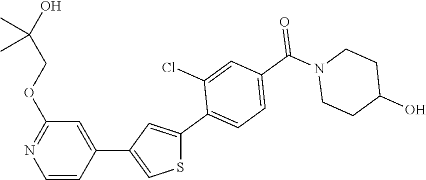 [SMILES: CC(C)(O)COc1cc(-c2csc(-c3ccc(C(=O)N4CCC(O)CC4)cc3Cl)c2)ccn1] | (3-chloro-4-(4-(2-(2-hydroxy-2-methylpropoxy)pyridin-4- yl)thiophen-2-yl)phenyl)(4-hydroxypiperidin-1-yl)methanone (Compound I-1629101) ¹H NMR (400 MHz, DMSO) δ 8.38 (d, J = 1.6 Hz, 1H), 8.17 (d, J = 5.4 Hz, 1H), 8.08 (d, J = 1.6 Hz, 1H), 7.86 (d, J = 8.0 Hz, 1H), 7.61 (d, J = 1.6 Hz, 1H), 7.44 (m, 1H), 7.39 (m, 1H), 7.24 (d, J = 0.8 Hz, 1H), 4.80 (d, J = 4.0 Hz, 1H), 4.62 (s, 1H), 4.07 (s, 2H), 3.98 (br s, 1H), 3.76 (m, 1H), 3.51 br s, 1H), 3.23 (m, 2H), 1.76 (m, 2H), 1.38 (m, 2H), 1.21 (s, 6H). LCMS: 99.72% ((M + H) ⁺ = 487.34) |
| 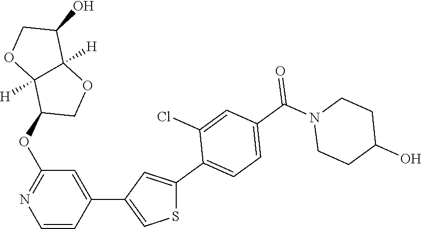 [SMILES: O=C(c1ccc(-c2cc(-c3ccnc(O[C@@H]4CO[C@H]5[C@@H]4OC[C@H]5O)c3)cs2)c(Cl)c1)N1CCC(O)CC1] | (3-chloro-4-(4-(2-(((3R,3aR,6R,6aR)-6- hydroxyhexahydrofuro [3,2-b]furan-3-yl)oxy)pyridin-4- yl)thiophen-2-yl)phenyl)(4-hydroxypiperidin-1-yl)methanone (Compound I-1629106) ¹H NMR (401 MHz, DMSO) δ 8.40 (d, J = 1.2 Hz, 1H), 8.18 (d, J = 5.2 Hz, 1H), 8.09 (d, J = 1.6 Hz, 1H), 7.85 (d, J = 8.0 Hz, 1H), 7.61 (d, J = 1.6 Hz, 1H), 7.43 (m, 2H), 7.29 (s, 1H), 5.36 (m, 1H), 4.91 (d, J = 6.8 Hz, 1H), 4.82 (d, J = 4.0 Hz, 1H), 4.77 (t, J = 4.8 Hz, 1H), 4.39 (t, J = 4.8 Hz, 1H), 4.14 (m, 2H), 3.98 (br s, 1H), 3.77 (q, J = 5.5 Hz, 3H), 3.50 (br s, 1H), 3.42 (m, 1H), 3.17 (m, 2H), 1.77 (m, 2H), 1.39 (br s, 2H). LCMS: 99.85% ((M + H) 543.34 |
| 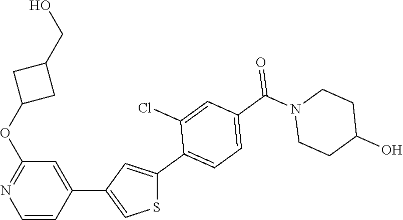 [SMILES: O=C(c1ccc(-c2cc(-c3ccnc(OC4CC(CO)C4)c3)cs2)c(Cl)c1)N1CCC(O)CC1] | (3-chloro-4-(4-(2-(3-(hydroxymethyl)cyclobutoxy)pyridin-4- yl)thiophen-2-yl)phenyl)(4-hydroxypiperidin-1-yl)methanone (I-1629110) ¹H NMR (400 MHz, DMSO) δ 8.36 (d, J = 1.6 Hz, 1H), 8.16 (dd, J = 2.4 Hz and 5.2 Hz, 1H), 8.06 (d, J = 1.6 Hz, 1H), 7.84 (d, J = 8.0 Hz, 1H), 7.61 (d, J = 1.6 Hz, 1H), 7.44 (d, J = 1.6 Hz and 8.0 Hz, 1H), 7.37 (dd, J = 1.6 Hz and 5.2 Hz, 1H), 7.19 (s, 1H), 5.08 (m, 1H), 4.81 (d, J = 4.0 Hz, 1H), 4.56 (t, J = 5.2 Hz, 1H), 3.99 (br s, 1H), 3.76 (m, 1H), 3.48 (m, 1H), 3.40 (t, J = 5.6 Hz, 2H), 3.21 (m, 2H), 2.44 (m, 2H), 2.10 (m, 1H), 1.81 (m, 4H), 1.40 (br s, 2H). LCMS: 99.29% ((M+H), 499.32). |
| 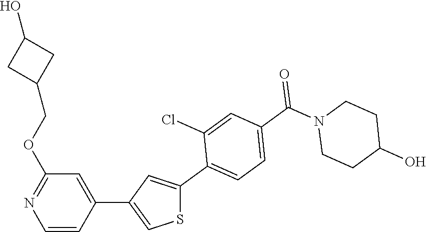 [SMILES: O=C(c1ccc(-c2cc(-c3ccnc(OCC4CC(O)C4)c3)cs2)c(Cl)c1)N1CCC(O)CC1] | (3-chloro-4-(4-(2-((3-hydroxycyclobutyl)methoxy)pyridin-4- yl)thiophen-2-yl)phenyl)(4-hydroxypiperidin-1-yl)methanone (I-1629118) ¹H NMR (401 MHz, DMSO) δ 8.38 (d, J = 1.2 Hz, 1H), 8.17 (d, J = 5.2 Hz, 1H), 8.08 (s, 1H), 7.85 (d, J = 8.0 Hz, 1H), 7.61 (d, J = 1.6 Hz, 1H), 7.44 (dd, J = 1.2 Hz and 8.0 Hz, 1H), 7.39 (m, 1H), 7.24 (d, J = 8.0 Hz, 1H), 5.02 (s, 1H), 4.81 (s, 1H), 4.26 (m, 3H), 3.99 (br s, 1.H), 3.76 (m, 1H), 3.51 (br s, 1H), 3.20 (br s, 2H), 2.32 (m, 1H), 2.05 (m, 3H), 1.71 (m, 3H), 1.39 (br s, 2H). LCMS: 99.55% ((M + H) 499.0). |
| 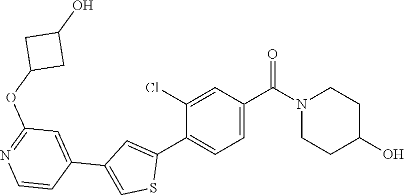 [SMILES: O=C(c1ccc(-c2cc(-c3ccnc(OC4CC(O)C4)c3)cs2)c(Cl)c1)N1CCC(O)CC1] | (3-chloro-4-(4-(2-(3-hydroxycyclobutoxy)pyridin-4- yl)thiophen-2-yl)phenyl)(4-hydroxypiperidin-1-yl)methanone (I-1629124) ¹H NMR (400 MHz, DMSO) δ 8.37 (d, J = 1.2 Hz, 1H), 8.16 (d, J = 5.4 Hz, 1H), 8.06 (d, J = 1.2 Hz, 1H), 7.84 (d, J = 8.0 Hz, 1H), 7.61 (d, J = 1.2 Hz, 1H), 7.44 (q, J = 3.2 Hz, 1H), 7.38 (q, J = 2.0 Hz, 1H), 7.21 (s, 1H), 5.17 (d, J = 6.4 Hz, 1H), 4.81 (d, J = 4.0 Hz, 1H), 4.75 (q, J = 7.2 Hz, 1H), 3.99 (s, 1H), 3.86 (q, J = 7.2 Hz, 1H), 3.76 (m, 1H), 3.51 (s, 1H), 3.20 (d, J = 6.4 Hz, 2H), 2.79 (m, 2H), 1.93 (m, 2H), 1.77 (m, 2H), 1.39 (m, 2H). LCMS: 96.41% ((M + H) 485.26). |

### Synthesis Example 9: 1-(3-chloro-4-(4-(2-(1-(hydroxymethyl)cyclopropyl)pyridin-4-yl)thiophen-2-yl)benzoyl)piperidin-4-yl L-valinate (Compound I-1629122)

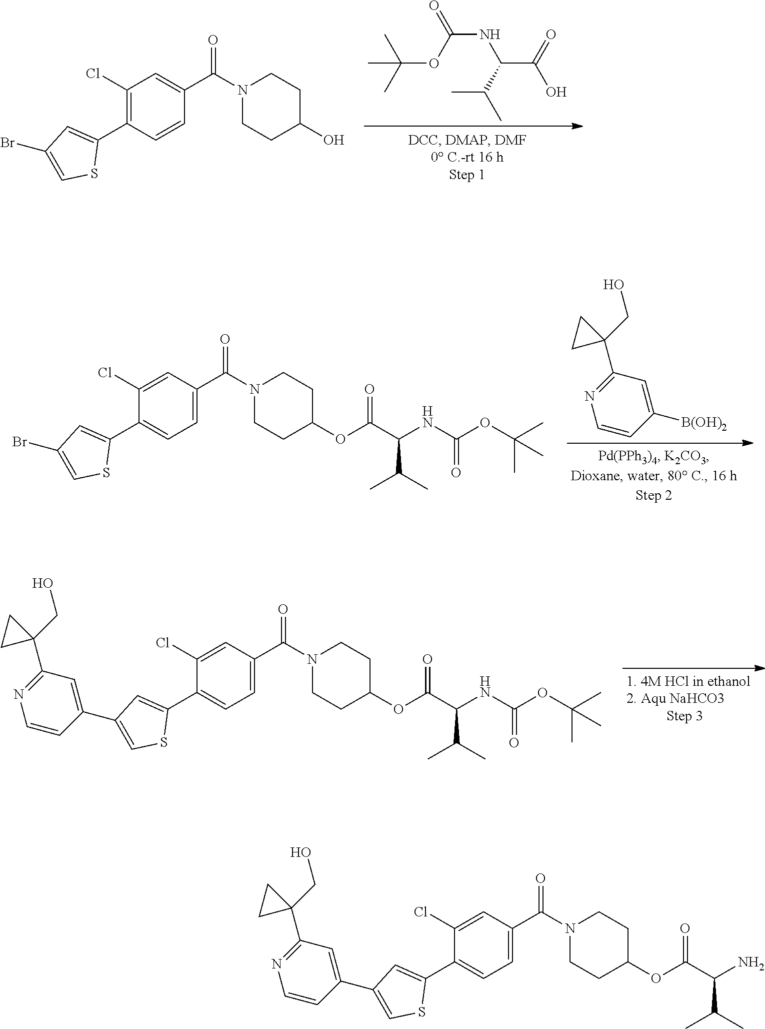 [SMILES: O=C(c1ccc(-c2cc(Br)cs2)c(Cl)c1)N1CCC(O)CC1 ; CC(C)[C@H](NC(=O)OC(C)(C)C)C(=O)O ; CC(C)[C@H](NC(=O)OC(C)(C)C)C(=O)OC1CCN(C(=O)c2ccc(-c3cc(Br)cs3)c(Cl)c2)CC1 ; OCC1(c2cc(B(O)O)ccn2)CC1 ; CC(C)[C@H](NC(=O)OC(C)(C)C)C(=O)OC1CCN(C(=O)c2ccc(-c3cc(-c4ccnc(C5(CO)CC5)c4)cs3)c(Cl)c2)CC1 ; CC(C)[C@H](N)C(=O)OC1CCN(C(=O)c2ccc(-c3cc(-c4ccnc(C5(CO)CC5)c4)cs3)c(Cl)c2)CC1]

Step 1: 1-(4-(4-bromothiophen-2-yl)-3-chlorobenzoyl)piperidin-4-yl (tert-butoxycarbonyl)-L-valinate. To a solution of (4-(4-bromothiophen-2-yl)-3-chlorophenyl)(4-hydroxypiperidin-1-yl)methanone (700 mg, 1.75 mmol), N-Boc L-valine (572 mg, 2.63 mmol) and DCC (724 mg, 3.51 mmol) in DMF (5 mL) was added DMAP (21 mg, 0.17 mmol). The reaction was stirred at rt for 16 h. and then diluted with ethyl acetate (30 mL), washed with water. The organic layer was dried over anhydrous over sodium sulfate and concentrated under reduced pressure to give a crude product which purified by reverse phase column chromatography to give 1-(4-(4-bromothiophen-2-yl)-3-chlorobenzoyl)piperidin-4-yl (tert-butoxycarbonyl)-L-valinate (600 mg; 60%) as an off white solid.

Step 2: 1-(3-chloro-4-(4-(2-(1-(hydroxymethyl)cyclopropyl)pyridin-4-yl)thiophen-2-yl)benzoyl)piperidin-4-yl (tert-butoxycarbonyl)-L-valinate. Coupling of the product of step 1 (600 mg; 1.0 mmol) with (2-(1-(hydroxymethyl)cyclopropyl)pyridin-4-yl)boronic acid (290 mg; 1.5 mmol) gave 1-(3-chloro-4-(4-(2-(1-(hydroxymethyl)cyclopropyl)pyridin-4-yl)thiophen-2-yl)benzoyl)piperidin-4-yl (tert-butoxycarbonyl)-L-valinate (250 mg; 37%) as a light brown solid.

Step 3: 1-(3-chloro-4-(4-(2-(1-(hydroxymethyl)cyclopropyl)pyridin-4-yl)thiophen-2-yl)benzoyl)piperidin-4-yl L-valinate (I-1629122). To a solution (250 mg, 0.52 mmol) in EtOH (5 mL), was added 4 M HCl in EtOH (4 mL) dropwise at 0° C. The reaction was stirred at rt for 2 h and then concentrated under reduced pressure. The residue was dissolved in water, the pH was adjusted 8-9 with aq.NaHCO3, and extracted with 10% MeOH in DCM. The combined organic layer was dried over Na₂SO₄ and concentrated to get crude which was purified by prep HPLC to give 1-(3-chloro-4-(4-(2-(1-(hydroxymethyl)cyclopropyl)pyridin-4-yl)thiophen-2-yl)benzoyl)piperidin-4-yl L-valinate (87 mg; 29%) as an off-white solid. 1H NMR (400 MHz, DMSO) δ 8.46 (d, J=5.2 Hz, 1H), 8.33 (d, J=1.2 Hz, 1H), 8.04 (d, J=1.2 Hz, 1H), 7.84 (d, J=8.0 Hz, 1H), 7.77 (s, 1H), 7.66 (d, J=1.2 Hz, 1H), 7.53 (q, J=2.0 Hz, 1H), 7.48 (q, J=3.2 Hz, 1H), 4.99 (m, 1H), 4.81 (t, J=5.6 Hz, 1H), 3.83 (d, J=5.6 Hz, 3H), 3.51 (s, 2H), 3.29 (s, 1H), 3.12 (d, J=5.2 Hz, 1H), 1.77 (m, 7H), 1.15 (q, J=3.2 Hz, 2H), 0.88 (m, 8H). LCMS: 99.92% (m/z=568.51 [M+H])⁺

The following compounds were prepared in a similar fashion to Synthesis Example 5 and Synthesis Example 9:

| | |
|---|---|
| 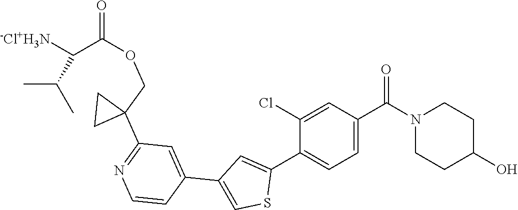 | (l-(4-(5-(2-chloro-4-(4-hydroxypiperidine-1- carbonyl)phenyl)thiophen-3-yl)pyridin-2- yl)cyclopropyl)methyl L-valinate (Compound I- 1629123) ¹H NMR (400 MHz, DMSO) δ 8.48 (d, J = 5.2 Hz, 1H), 8.35 (d, J = 1.2 Hz, 1H), 8.04 (d, J = 1.2 Hz, 1H), 7.82 (d, J = 8.0 Hz, 1H), 7.69 (s, 1H), 7.62 (d, J = 1.6 Hz, 1H), 7.57 (q, J = 2.0 Hz, 1H), 7.45 (q, J = 3.2 Hz, 1H), 4.81 (d, J = 3.6 Hz, 1H), 4.64 (d, J = 12 Hz, 1H), 4.42 (d, J = 12 Hz, 1H), 3.99 (s, 1H), 3.76 (d, J = 3.6 Hz, 1H), 3.51 (s, 1H), 3.22 (d, J = 3.2 Hz, 2H), 3.09 (d, J = 5.2 Hz, 1H), 1.71 (m, 5H), 1.32 (m, 4H), 1.07 (d, J = 2.0 Hz, 2H), 0.78 (d, J = 6.8 Hz, 3H), 0.72 (d, J = 6.8 Hz, 3H). LCMS: 98.22% (m/z = 568.25 [M + H]) ⁺ion present) |

### Synthesis Example 10: (3-chloro-4-(4-(2-((1-hydroxycyclobutyl)methyl)pyridin-4-yl)thiophen-2-yl)phenyl)(4-hydroxypiperidin-1-yl)methanone (I-1629077)

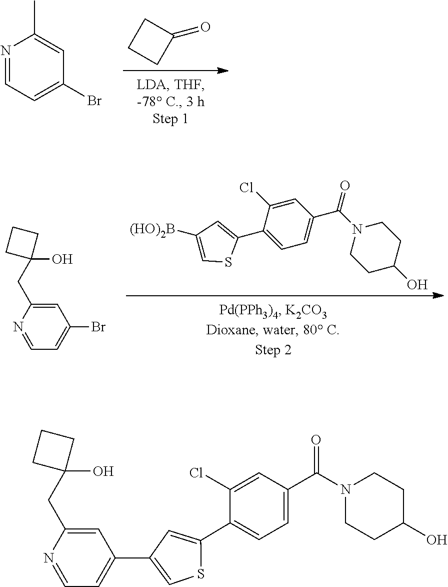

Step 1: 1-((4-bromopyridin-2-yl)methyl)cyclobutan-1-ol). To a freshly prepared solution of LDA in 10 ml of THF (DIPA 0.88 g; 8.72 mmol; n-BuLi 0.82 g; 12.79 mmol) at −50° C. was added 4-bromo-1 methylpyridine (1.0 g, 5.81 mmol) in THE (2 mL). The mixture was stirred for 15 min and then cyclobutanone was added (0.896 g, 12.79 mmol) and stirred for another 15 min. After completion of the reaction, reaction mixture was quenched by adding aq. sat NH₄Cl and extracted with EtOAc (3×50 mL). The combined extracts were dried over anhydrous sodium sulfate and concentrated under reduced pressure to afford crude product which was purified by column chromatography to give 1-((4-bromopyridin-2-yl)methyl)cyclobutan-1-ol) as colorless viscous liquid.

Step 2: (3-chloro-4-(4-(2-((1-hydroxycyclobutyl)methyl)pyridin-4-yl)thiophen-2-yl)phenyl)(4-hydroxypiperidin-1-yl)methanone (I-1629077). To a solution of 1-((4-bromopyridin-2-yl)methyl)cyclobutan-1-ol) (200 mg, 0.82 mmol, 1 eq) and (5-(2-chloro-4-(4-hydroxypiperidine-1-carbonyl)phenyl)thiophen-3-yl)boronic acid (556 mg, 1.24 mmol) in 1,4-dioxane:water (8 mL:2 mL) was added K₂CO₃ (343 mg, 2.48 mmol). After purging with nitrogen for 5 minutes tetrakis palladium (95 mg, 0.08 mmol) was added and purged again for 5 minutes. The reaction mixture was stirred at 80° C. for 16 h and filtered through celite bed with ethyl acetate washing. The filtrate and washings were combined and concentrated under reduced pressure to give a crude product which was purified by Prep-HPLC to give (3-chloro-4-(4-(2-((1-hydroxycyclobutyl)methyl)pyridin-4-yl)thiophen-2-yl)phenyl)(4-hydroxypiperidin-1-yl)methanone (155 mg; 39%) as an off white fluffy solid. ¹H NMR (400 MHz, DMSO): δ 8.51 (d, J=5.2 Hz, 1H), 8.32 (d, J=1.6 Hz, 1H), 8.02 (d, J=1.6 Hz, 1H), 7.83 (d, J=8.0 Hz, 1H), 7.73 (d, J=0.8 Hz, 1H), 7.63 (m, 2H), 7.45 (dd, J=1.6 Hz and 8.0 Hz, 1H), 5.31 (s, 1H), 4.81 (d, J=3.6 Hz, 1H), 3.99 (br s, 1H), 3.76 (m, 1H), 3.52 (br s, 1H), 3.19 (br s, 2H), 3.00 (s, 2H), 2.13 (m, 2H), 1.95 (m, 2H), 1.77 (m, 2H), 1.62 (t, J=5.4 Hz, 1H), 1.42 (m, 3H). LCMS: 99.71% (m/z=483.37 [M+H])⁺.

The following compound is prepared in a similar fashion to Synthesis Example 5 and Synthesis Example 9.

| | |
|---|---|
| 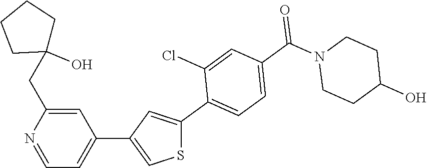 [SMILES: O=C(c1ccc(-c2cc(-c3ccnc(CC4(O)CCCC4)c3)cs2)c(Cl)c1)N1CCC(O)CC1] | (3-chloro-4-(4-(2-((1-hydroxycyclopentyl)methyl)pyridin- 4-yl)thiophen-2-yl)phenyl)(4-hydroxypiperidin-1- yl)methanone |

### Synthesis Example 11: (3-chloro-4-(4-(2-((1-fluorocyclobutyl)methyl)pyridin-4-yl)thiophen-2-yl)phenyl)(4-hydroxypiperidin-1-yl)methanone (I-1629078)

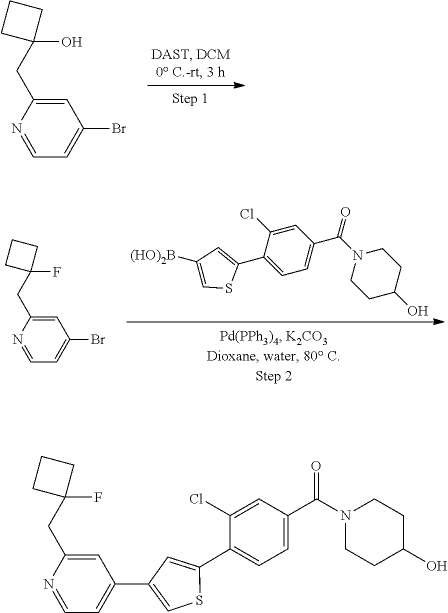 [SMILES: OC1(Cc2cc(Br)ccn2)CCC1 ; FC1(Cc2cc(Br)ccn2)CCC1 ; O=C(c1ccc(-c2cc(B(O)O)cs2)c(Cl)c1)N1CCC(O)CC1 ; O=C(c1ccc(-c2cc(-c3ccnc(CC4(F)CCC4)c3)cs2)c(Cl)c1)N1CCC(O)CC1]

Step 1: 4-bromo-2-((1-fluorocyclobutyl)methyl)pyridine. To a stirred solution of 1-((4-bromopyridin-2-yl)methyl)cyclobutan-1-ol (200 mg, 0.82 mmol) in DCM (5 mL) was added DAST (267 mg, 1.65 mmol) at −78° C. The reaction mass was stirred at rt for 3 h and then quenched with NaHCO₃ and the product was extracted with DCM. The combined extracts were dried over anhydrous sodium sulfate and concentrated under (low temperature at 35° C.) reduced pressure to afford crude product which was purified by column chromatography to give 4-bromo-2-((1-fluorocyclobutyl)methyl)pyridine (100 mg; 50%) as a light yellow liquid.

Step 2: (3-chloro-4-(4-(2-((1-fluorocyclobutyl)methyl)pyridin-4-yl)thiophen-2-yl)phenyl)(4-hydroxypiperidin-1-yl)methanone. The product of Step 1 was coupled with (5-(2-chloro-4-(4-hydroxypiperidine-1-carbonyl)phenyl)thiophen-3-yl)boronic acid (275 mg, 0.61 mmol) to give (3-chloro-4-(4-(2-((1-fluorocyclobutyl)methyl)pyridin-4-yl)thiophen-2-yl)phenyl)(4-hydroxypiperidin-1-yl)methanone (15 mg; 39%) as a white fluffy solid. ¹H NMR (400 MHz, DMSO): δ 8.55 (d, J=5.2 Hz, 1H), 8.35 (d, J=1.6 Hz, 1H), 8.05 (d, J=1.6 Hz, 1H), 7.84 (d, J=8.0 Hz, 1H), 7.71 (s, 1H), 7.67 (dd, J=1.6 Hz and 5.2 Hz, 1H), 7.62 (d, J=1.6 Hz, 1H), 7.45 (dd, J=1.6 Hz and 8.0 Hz, 1H), 4.81 (d, J=4.0 Hz, 1H), 3.99 (br s, 1H), 3.75 (m, 1H), 3.50 (br s, 1H), 3.21 (d, J=23.2 Hz, 4H), 2.37 (m, 2H), 2.23 (m, 2H), 1.76 (m, 3H), 1.45 (m, 3H). LCMS: 99.03% (m/z=485.34 [M+H])⁺

### Synthesis Example 12: (3-chloro-4-(4-(3-(hydroxymethyl)-2,3-dihydro-[1,4]dioxino[2,3-b]pyridin-8-yl)thiophen-2-yl)phenyl)(4-hydroxypiperidin-1-yl)methanone (I-1629181)

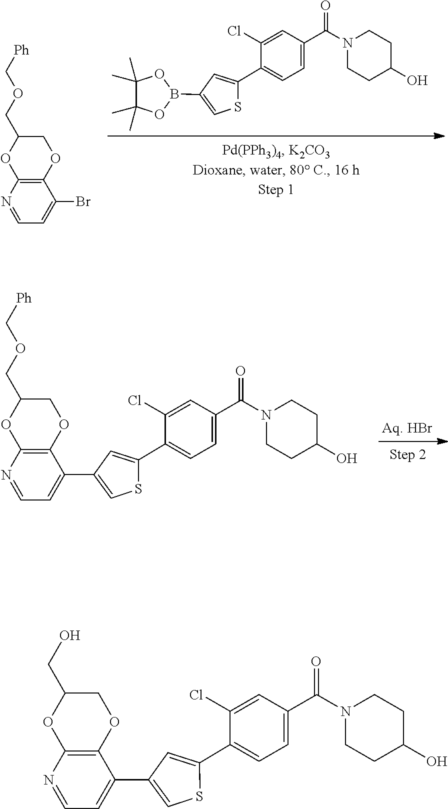 [SMILES: Brc1ccnc2c1OCC(COCc1ccccc1)O2 ; CC1(C)OB(c2csc(-c3ccc(C(=O)N4CCC(O)CC4)cc3Cl)c2)OC1(C)C ; O=C(c1ccc(-c2cc(-c3ccnc4c3OCC(COCc3ccccc3)O4)cs2)c(Cl)c1)N1CCC(O)CC1 ; O=C(c1ccc(-c2cc(-c3ccnc4c3OCC(CO)O4)cs2)c(Cl)c1)N1CCC(O)CC1]

Step 1: (4-(4-(3-((benzyloxy)methyl)-2,3-dihydro-[1,4]dioxino[2,3-b]pyridin-8-yl)thiophen-2-yl)-3-chlorophenyl)(4-hydroxypiperidin-1-yl)methanone. A mixture of K₂CO₃ (0.145 g, 1.05 mmol) 3-((benzyloxy)methyl)-8-bromo-2,3-dihydro-[1,4]dioxino[2,3-b]pyridine (0.12 g, 0.35 mmol) and (3-chloro-4-(4-(4,4,5,5-tetramethyl-1,3,2-dioxaborolan-2-yl)thiophen-2-yl)phenyl)(4-hydroxypiperidin-1-yl)methanone (as prepared in Example 1) (0.15 g, 0.42 mmol) in 1,4-dioxane:water (5 mL: 1 mL) was purged with nitrogen gas for 5 minutes. Palladium tetrakis (0.021 g, 0.017 mmol) was added and the reaction mixture was stirred at 80° C. for 16 h. The mixture was cooled and filtered through a celite bed and washed with ethyl acetate. The combined filtrates were concentrated under reduced pressure to afford crude product which was purified by reverse phase column chromatography to (4-(4-(3-((benzyloxy)methyl)-2,3-dihydro-[1,4]dioxino[2,3-b]pyridin-8-yl)thiophen-2-yl)-3-chlorophenyl)(4-hydroxypiperidin-1-yl)methanone (0.325 mg) as a brown gum which was used in the next step.

Step 2: (3-chloro-4-(4-(3-(hydroxymethyl)-2,3-dihydro-[1,4]dioxino[2,3-b]pyridin-8-yl)thiophen-2-yl)phenyl)(4-hydroxypiperidin-1-yl)methanone. A solution of the product os Step 1 (0.11 g, 0.19 mmol) in aq. HBr (47%) (4 mL) was stirred at rt for 4 h. The mixture was then diluted with ethyl acetate (50 mL) and washed with sat. NaHCO₃ solution. The organic layer was dried over anhydrous sodium sulfate and concentrated under reduced pressure to obtain a crude product which was purified by prep. HPLC to give racemic (3-chloro-4-(4-(3-(hydroxymethyl)-2,3-dihydro-[1,4]dioxino[2,3-b]pyridin-8-yl)thiophen-2-yl)phenyl)(4-hydroxypiperidin-1-yl)methanone as a white solid. (51 mg, 50% yield). ¹H NMR (400 MHz, DMSO): δ 8.25 (d, J=1.6 Hz, 1H), 7.96 (d, J=1.6 Hz, 1H), 7.79 (s, 1H), 7.78 (d, J=3.2 Hz, 1H), 7.61 (d, J=1.6 Hz, 1H), 7.43 (dd, J=1.6, 8.0 Hz, 1H), 7.32 (d, J=4.8 Hz, 1H), 5.18 (brs, 1H), 4.75 (brs, 1H), 4.48-4.39 (m, 2H), 4.14-4.10 (m, 1H), 3.98 (brs, 1H), 3.77-3.66 (m, 3H), 3.51 (brs, 1H), 3.25-3.20 (m, 2H), 1.79-1.74 (m, 2H), 1.39 (brs, 2H). LCMS: 97.3% (M+H=487.42)

Step 3: Separation of enantiomers of (3-chloro-4-(4-(3-(hydroxymethyl)-2,3-dihydro-[1,4]dioxino[2,3-b]pyridin-8-yl)thiophen-2-yl)phenyl)(4-hydroxypiperidin-1-yl)methanone. Racemic (3-chloro-4-(4-(3-(hydroxymethyl)-2,3-dihydro-[1,4]dioxino[2,3-b]pyridin-8-yl)thiophen-2-yl)phenyl)(4-hydroxypiperidin-1-yl)methanone (I-1629181) was separated into the two pure enantiomers using a chiral Lux Cellulose-4 column:

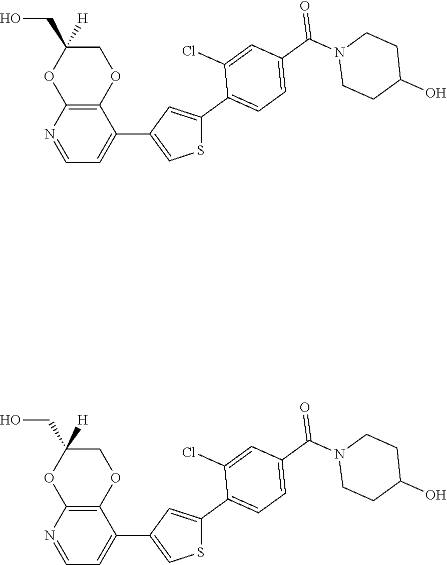 [SMILES: O=C(c1ccc(-c2cc(-c3ccnc4c3OC[C@H](CO)O4)cs2)c(Cl)c1)N1CCC(O)CC1 ; O=C(c1ccc(-c2cc(-c3ccnc4c3OC[C@@H](CO)O4)cs2)c(Cl)c1)N1CCC(O)CC1]

Characterization for the two separated enantiomers is as follows:

Rt: 14.66 min; ee: 99.21%; Specific optical rotation [α]²⁵₅₈₉: +29.74
(c 0.5, MeOH)
1H NMR (400 MHz, DMSO) δ 8.25 (d, J = 1.6 Hz, 1H), 7.96
(d, J = 1.2 Hz, 1H), 7.79 (s, 1H), 7.78 (d,
J = 2.8 Hz, 1H), 7.61 (d, J = 1.2 Hz, 1H), 7.44-7.42
(dd, J = 1.2 Hz and 8.0 Hz, 1H), 7.31 (d, J = 5.2 Hz, 1H), 5.20-5.15
(m, 1H), 4.80 (d, J = 3.2 Hz, 1H), 4.50-4.35 (m, 2H), 4.15-4.10
(m, 1H), 4.05-3.95 (br s, 1H), 3.80-3.65 (m, 3H), 3.60-3.45
(br s, 1H), 3.30-3.15 (m, 2H), 1.85-1.65 (m, 2H), 145-1.30 (m, 2H)
Rt: 16.91 min; ee: 95.90%; Specific optical rotation [α]²⁵₅₈₉: −23.45
(c 0.5, MeOH)
1H NMR (400 MHz, DMSO): δ 8.25 (d, J = 1.2 Hz, 1H), 7.96
(d, J = 1.2 Hz, 1H), 7.79 (s, 1H), 7.78 (d,
J = 2.8 Hz, 1H), 7.61 (d, J = 1.2 Hz, 1H), 7.44-7.42
(dd, J = 1.6 Hz and 8.0 Hz, 1H), 7.31 (d, J = 5.2 Hz, 1H), 5.20-5.15
(m, 1H), 4.80 (d, J = 3.6 Hz, 1H), 4.50-4.35 (m, 2H), 4.15-4.10
(m, 1H), 4.05-3.95 (br s, 1H), 3.80-3.65 (m, 3H), 3.60-3.45
(br s, 1H), 3.30-3.15 (m, 2H), 1.85-1.65 (m, 2H), 145-1.30 (m, 2H).

### Synthesis Example 13: (3-chloro-4-(4-(2-cyclobutoxypyridin-4-yl)thiophen-2-yl)phenyl)(4-hydroxypiperidin-1-yl)methanone (I-629257)

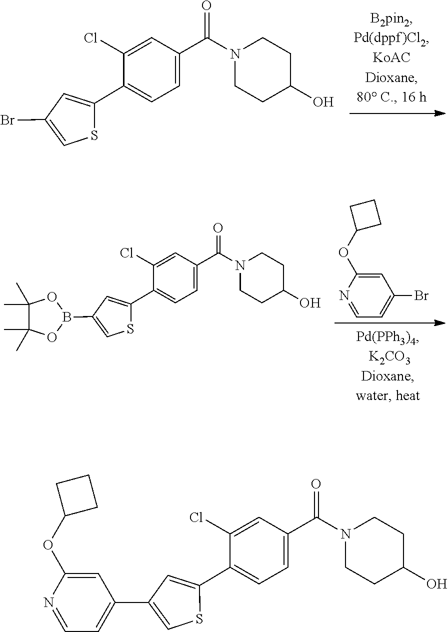

Step 1: (3-chloro-4-(4-(4,4,5,5-tetramethyl-1,3,2-dioxaborolan-2-yl)thiophen-2-yl)phenyl)(4-hydroxypiperidin-1-yl)methanone. A stirred solution of (4-(4-bromothiophen-2-yl)-3-chlorophenyl)(4-hydroxypiperidin-1-yl)methanone (1 g, 2.51 mmol), B₂pin₂ (0.95 mg, 3.76 mmol) and potassium acetate (0.73 g, 7.53 mmol) in 1,4-dioxane (20 mL) in a 48 ml glass tube was purged with nitrogen for 10 min. PdCl₂(dppf) (0.092 g, 0.0681 mmol) was added and the reaction mixture again purged with nitrogen for 20 minutes. The tube was sealed and heated at 80° C. for 16 h. After cooling to RT the reaction mixture was filtered through a celite bed and concentrated under reduced pressure to afford (3-chloro-4-(4-(4,4,5,5-tetramethyl-1,3,2-dioxaborolan-2-yl)thiophen-2-yl)phenyl)(4-hydroxypiperidin-1-yl)methanone (1.5 g) as a brown gum which was used in the next without further purification.

Step 2: (3-chloro-4-(4-(2-cyclobutoxypyridin-4-yl)thiophen-2-yl)phenyl)(4-hydroxypiperidin-1-yl)methanone. A suspension of the crude material from the previous step (0.74 g, 1.65 mmol), 4-bromo-2-cyclobutoxypyridine (0.25 g, 1.10 mmol), and K₂CO₃ (0.455 g, 3.3 mmol) in 1,4-Dioxane/water (6 mL) in a 48 mL glass tube was purged with nitrogen gas for 20 minutes. After adding Pd(PPh₃)₄ (0.045 g, 0.055 mmol) the tube was sealed and the reaction mass was heated at 80° C. for 16 h. The reaction mixture was filtered through celite, concentrated under reduced pressure to obtain crude and purified by reverse phase column chromatography to give (3-chloro-4-(4-(2-cyclobutoxypyridin-4-yl)thiophen-2-yl)phenyl)(4-hydroxypiperidin-1-yl)methanone as a fluffy white solid (0.25 g, 45%). ¹HNMR (400 MHz, DMSO-d6): δ 8.36 (s, 1H), 8.16 (d, J=5.20 Hz, 1H), 8.06 (s, 1H), 7.84 (d, J=7.60 Hz, 1H), 7.61 (s, 1H), 7.44 (d, J=8.00 Hz, 1H), 7.38 (d, J=5.20 Hz, 1H), 7.20 (s, 1H), 5.22-5.14 (m, 1H), 4.79 (d, J=3.60 Hz, 1H), 3.99 (br s, 1H), 3.78-3.77 (m, 1H), 3.55-3.45 (m, 1H), 3.30-3.15 (m, 2H), 2.50-2.40 (m, 2H), 2.15-2.00 (m, 2H), 1.85-1.60 (m, 4H), 1.45-1.30 (m, 2H). LCMS: 99.56% (m/z=469.23[M+H])

The following compound was prepared in a similar fashion to Synthesis Example 13:

| | |
|---|---|
| 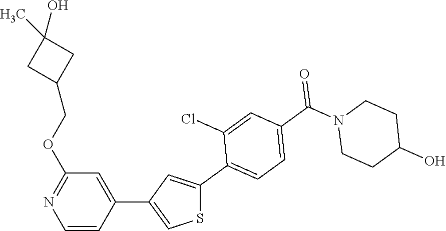 [SMILES: CC1(O)CC(COc2cc(-c3csc(-c4ccc(C(=O)N5CCC(O)CC5)cc4Cl)c3)ccn2)C1] | (3-chloro-4-(4-(2-((3-hydroxy-3- methylcyclobutyl)methoxy)pyridin-4-yl)thiophen-2- yl)phenyl)(4-hydroxypiperidin-1-yl)methanone MS: m/z 513.33 (M + H). |

The following compound is prepared in a similar fashion to Synthesis Example 13:

| | |
|---|---|
| 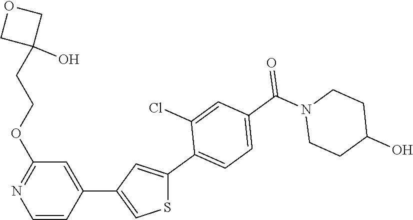 [SMILES: O=C(c1ccc(-c2cc(-c3ccnc(OCCC4(O)COC4)c3)cs2)c(Cl)c1)N1CCC(O)CC1] | (3-chloro-4-(4-(2-(2-(3-hydroxyoxetan-3-yl)ethoxy)pyridin-4- yl)thiophen-2-yl)phenyl)(4-hydroxypiperidin-1-yl)methanone |

### Biological Example 1: Gene Expression, Reporter Assay, and Liver Microsomes

Gene Expression: The effect of selected compounds on the gene expression of HepG2 cells was evaluated. HepG2 cells (P2) were seeded in 24 well plate (80,000 cells/well) for RNA extraction and in a 96 well plate (10,000 cells/well) for Cell Titer Glow (CTG). The media used was DMEM and contained 10% FBS. Each compound was evaluated at 500 Mm for 48 hours. Two biological replicates per experimental group were evaluated for RNA. For gene analysis, RNA was harvested with RNEasy kit and 20-100 ng used to synthesize cDNA with random primers. Quantitative PCR was performed on 1 pg to 100 ng cDNA for the following genes: ACACA, ACLY, FASN, LSS, PNPLA3. Gene expression levels were normalized with housekeeping gene, β-Actin, and relative expression levels determined using ΔΔCT method comparing treated to mock or vehicle treated cells as a baseline. “Total” gene expression refers to the average of the values of the 5 genes listed above. Results are presented in Table 1 below.

Reporter Screening Assay: This assay was used to evaluate the effect on transcriptional activity SREBP of selected compounds using an SRE-luciferase reporter construct. On day 1, 10,000 cells were seeded in a 96 well (white) plate as per the plate map in Growth media without antibiotics. Cells were incubated at 37° C. for 8 hours. After 8 hours, cells were washed with DPBS for complete removal of FBS. DPBS was completely removed and Growth media was replaced with phenol free treatment medium (90 μl) with different FBS concentrations. The cells were then incubated at 37° C. for 24 hours with varying doses (0.01 uM to 10 uM) of compounds. Then a Luciferase assay was performed.

Reagents for performing Luciferase assay were stored at −20° C. To a tube of lyophilized assay substrate was added 1 mL Substrate Solvent and mixed well. The Substrate tube after reconstitution was covered with aluminum foil so as to keep it protected from light. The assay buffer was thawed to room temperature. To 20 mL Assay Buffer was added 200 L of reconstituted 100× Substrate and mixed well. The reconstituted substrate as well as the assay solution (buffer + substrate) was protected from light throughout the procedure by keeping it covered with aluminum foil. Using a multi-channel pipette, 100 μL Assay Solution (buffer + substrate) was added directly to each sample well in Plate 1, which was incubated for 30 min (plate was covered with aluminum foil). After 30 min incubation, the plate was read for total luminescence. Each well was read for 2 seconds in a plate luminometer. (Microplate reader Envision Microplate reader from Perkin Elmer). Precaution was taken to incubate plate exactly for 30 min prior to reading on the plate reader. Results are presented in Table 1 below.

Reporter Assay Materials: SREBPv1 Reporter cell line: HepG2-#32251. Growth Medium: MEM (Corning 10-010), 10% FBS, 1% GlutaMax (Invitrogen Catalog #35050061), μg/ml Puromycin (Invitrogen Catalog #A1113803) and 1% Penicillin-Streptomycin (Pen-Strep). Treatment Media: Phenol-free MEM (Invitrogen Catalog #51200-038) and 1% GlutaMax (Invitrogen Catalog #35050061). Luciferase Assay: LightSwitch Luciferase Assay Kit (Catalog #32032). LDH assay: Pierce LDH Cytotoxicity Assay Kit (Catalog #SD249616).

Half-life Human Microsomes: Compounds were evaluated for stability in human liver microsomes. A 10 mM stock solution of the compound being evaluated was prepared in DMSO and diluted with water:acetonitrile (1:1) to a concentration of 1 mM. A working concentration of 100 μM was prepared by further dilution with water:acetonitrile (1:1). To make the preincubation mixture, 2.5 μL of the diluted compound was combined with 75 μL of human liver microsomes at 3.33 mg/mL, and 85 μL of 100 mM potassium phosphate buffer, and this mixture was pre-incubated for 10 min at 37° C. To make the 60 minute mixture without cofactor, 32.5 μL of the preincubation mixture was combined with 17.5 μL of 100 mM potassium phosphate buffer and incubated for 60 min at 37° C. To make the 0 min sample with cofactor (NADPH), 16.25 μL of the preincubation mixture was combined with 200 μL of acetonitrile containing internal standard and 8.75 μL of cofactor (NADPH). To make the incubation mixture, 62 μL of cofactor (2.85 mM) was combined with the remaining incubation mixture, and incubated for 60 min at 37° C. To prepare the sample mixture to be evaluated, 25 μL incubation mixture was combined with 200 μL of acetonitrile containing internal standard and vortexed for 5 min at 1200 rpm, then centrifuged for 10 min at 4000 rpm. The supernatant was diluted 2 fold with water and injected on LC-MS/MS. The sample mixture was evaluated by LC-MS/MS using 10 mM ammonium acetate with 0.1% FA as the aqueous mobile phase, and methanol as the organic mobile phase.

Half-life Mouse Microsomes: Compounds were evaluated in mouse liver microsomes following a similar procedure as described above for human liver microsomes. A similar procedure could be used to evaluate compounds in rat liver microsomes. Results are presented in Table 1 below.

TABLE 1
Data for selected compounds. Entries A-F are effect of compounds on gene
expression of HepG2 cells. A: ACACA; B: ACLY; C: FASN; D: LSS; E: PNPLA3;
F: Total. For gene expression at the tested dose, 0-0.309 = +++, 0.31-0.7509 = ++,
>0.751 = +. Entries G and H are the half-life of liver microsomes (percent rem @ 60
min; G is human, H is mouse), where 0-30 = +; 30.1-60 = ++; ≥60.1 = +++. Entry I
is for the Reporter Screening Assay (Ave EC50 (nM)), where 0-249 = +++, 250-500 =
++, and >500 = +.

| Compound | A | B | C | D | E | F | G | H | I |
|---|---|---|---|---|---|---|---|---|---|
| 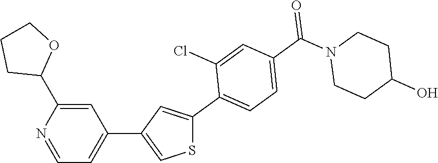 [SMILES: O=C(c1ccc(-c2cc(-c3ccnc(C4CCCO4)c3)cs2)c(Cl)c1)N1CCC(O)CC1] | ++ | ++ | ++ | +++ | +++ | ++ | + | +++ | +++ |
| I-1628743 | | | | | | | | | |
| 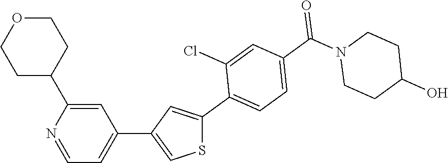 [SMILES: O=C(c1ccc(-c2cc(-c3ccnc(C4CCOCC4)c3)cs2)c(Cl)c1)N1CCC(O)CC1] | + | ++ | ++ | + | ++ | ++ | + | +++ | + |
| I-1628752 | | | | | | | | | |
| 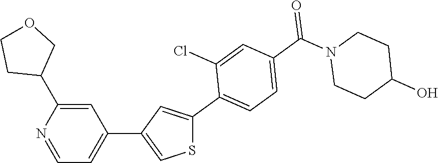 [SMILES: O=C(c1ccc(-c2cc(-c3ccnc(C4CCOC4)c3)cs2)c(Cl)c1)N1CCC(O)CC1] | ++ | ++ | ++ | +++ | +++ | +++ | + | +++ | +++ |
| I-1628763 | | | | | | | | | |
| 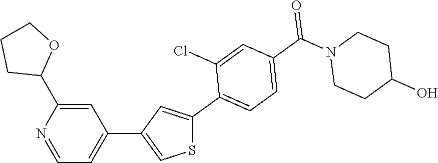 [SMILES: O=C(c1ccc(-c2cc(-c3ccnc(C4CCCO4)c3)cs2)c(Cl)c1)N1CCC(O)CC1] | ++ | ++ | ++ | ++ | +++ | ++ | + | +++ | ++ |
| I-1628777 | | | | | | | | | |
| 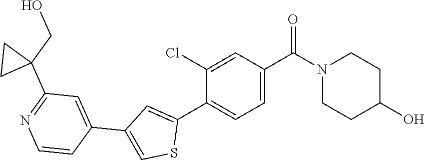 [SMILES: O=C(c1ccc(-c2cc(-c3ccnc(C4(CO)CC4)c3)cs2)c(Cl)c1)N1CCC(O)CC1] | ++ | ++ | ++ | +++ | +++ | ++ | +++ | +++ | +++ |
| I-1628961 | | | | | | | | | |
| 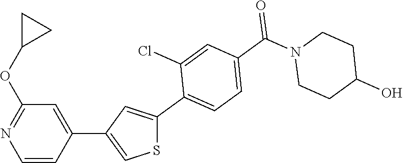 [SMILES: O=C(c1ccc(-c2cc(-c3ccnc(OC4CC4)c3)cs2)c(Cl)c1)N1CCC(O)CC1] | ++ | ++ | ++ | +++ | +++ | ++ | +++ | +++ | +++ |
| I-1629059 | | | | | | | | | |
| 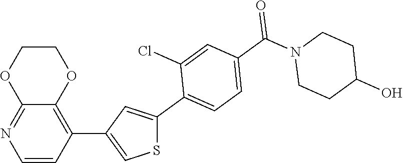 [SMILES: O=C(c1ccc(-c2cc(-c3ccnc4c3OCCO4)cs2)c(Cl)c1)N1CCC(O)CC1] | ++ | ++ | ++ | +++ | +++ | ++ | ++ | ++ | +++ |
| I-1629066 | | | | | | | | | |
| 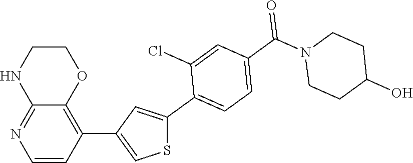 [SMILES: O=C(c1ccc(-c2cc(-c3ccnc4c3OCCN4)cs2)c(Cl)c1)N1CCC(O)CC1] | ++ | ++ | ++ | ++ | +++ | ++ | + | + | +++ |
| I-1629067 | | | | | | | | | |
| 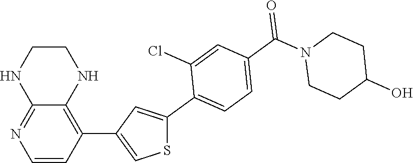 [SMILES: O=C(c1ccc(-c2cc(-c3ccnc4c3NCCN4)cs2)c(Cl)c1)N1CCC(O)CC1] | ++ | ++ | ++ | +++ | +++ | ++ | ++ | + | +++ |
| I-1629068 | | | | | | | | | |
| 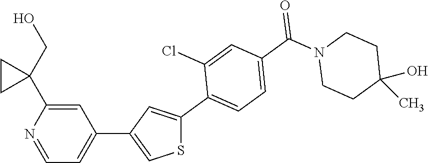 [SMILES: CC1(O)CCN(C(=O)c2ccc(-c3cc(-c4ccnc(C5(CO)CC5)c4)cs3)c(Cl)c2)CC1] | + | + | + | + | + | + | + | + | + |
| I-1629069 | | | | | | | | | |
| 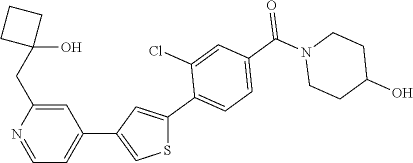 [SMILES: O=C(c1ccc(-c2cc(-c3ccnc(CC4(O)CCC4)c3)cs2)c(Cl)c1)N1CCC(O)CC1] | ++ | + | ++ | +++ | +++ | ++ | + | ++ | +++ |
| I-1629077 | | | | | | | | | |
| 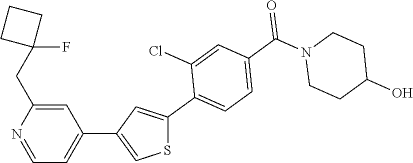 [SMILES: O=C(c1ccc(-c2cc(-c3ccnc(CC4(F)CCC4)c3)cs2)c(Cl)c1)N1CCC(O)CC1] | ++ | + | +++ | +++ | ++ | ++ | + | +++ | +++ |
| I-1629078 | | | | | | | | | |
| 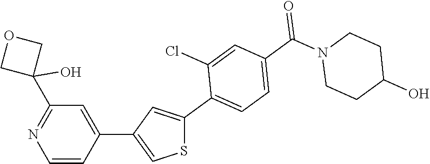 [SMILES: O=C(c1ccc(-c2cc(-c3ccnc(C4(O)COC4)c3)cs2)c(Cl)c1)N1CCC(O)CC1] | + | + | ++ | ++ | ++ | ++ | +++ | +++ | ++ |
| I-1629083 | | | | | | | | | |
| 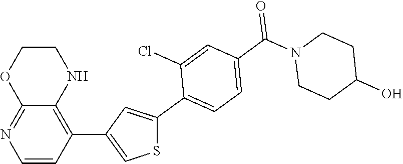 [SMILES: O=C(c1ccc(-c2cc(-c3ccnc4c3NCCO4)cs2)c(Cl)c1)N1CCC(O)CC1] | ++ | ++ | ++ | +++ | +++ | ++ | + | ++ | +++ |
| I-1629084 | | | | | | | | | |
| 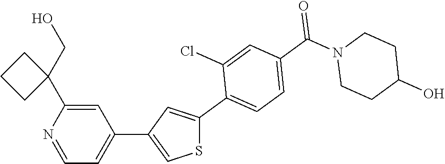 [SMILES: O=C(c1ccc(-c2cc(-c3ccnc(C4(CO)CCC4)c3)cs2)c(Cl)c1)N1CCC(O)CC1] | | | | | | | + | +++ | + |
| I-1629089 | | | | | | | | | |
| 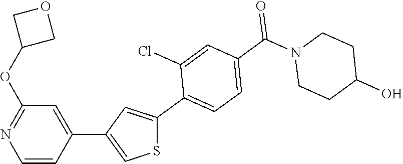 [SMILES: O=C(c1ccc(-c2cc(-c3ccnc(OC4COC4)c3)cs2)c(Cl)c1)N1CCC(O)CC1] | | | | | | | +++ | +++ | + |
| I-1629100 | | | | | | | | | |
| 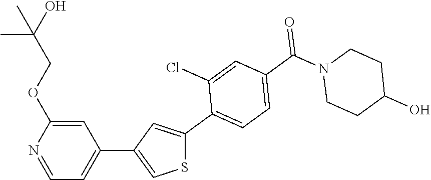 [SMILES: CC(C)(O)COc1cc(-c2csc(-c3ccc(C(=O)N4CCC(O)CC4)cc3Cl)c2)ccn1] | | | | | | | +++ | +++ | + |
| I-1629101 | | | | | | | | | |
| 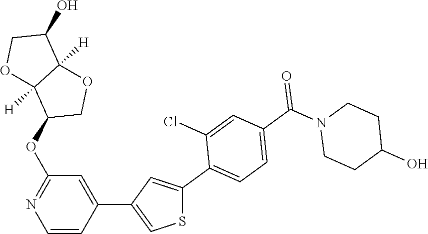 [SMILES: O=C(c1ccc(-c2cc(-c3ccnc(O[C@@H]4CO[C@H]5[C@@H]4OC[C@H]5O)c3)cs2)c(Cl)c1)N1CCC(O)CC1] | | | | | | | +++ | +++ | +++ |
| I-1629106 | | | | | | | | | |
| 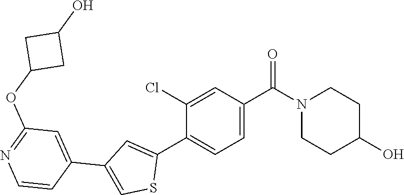 [SMILES: O=C(c1ccc(-c2cc(-c3ccnc(OC4CC(O)C4)c3)cs2)c(Cl)c1)N1CCC(O)CC1] | | | | | | | | +++ | +++ |
| I-1629124 | | | | | | | | | |
| 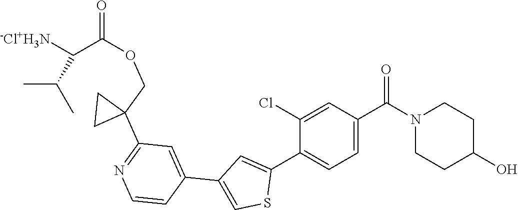 | | | | | | | | | +++ |
| I-1629123 | | | | | | | | | |
| 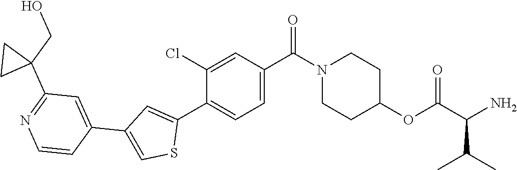 [SMILES: CC(C)[C@H](N)C(=O)OC1CCN(C(=O)c2ccc(-c3cc(-c4ccnc(C5(CO)CC5)c4)cs3)c(Cl)c2)CC1] | | | | | | | | | +++ |
| I-1629122 | | | | | | | | | |
| 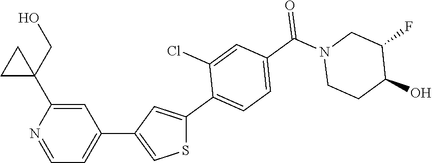 [SMILES: O=C(c1ccc(-c2cc(-c3ccnc(C4(CO)CC4)c3)cs2)c(Cl)c1)N1CC[C@H](O)[C@@H](F)C1] | | | | | | | | +++ | +++ |
| I-1629121 | | | | | | | | | |
| 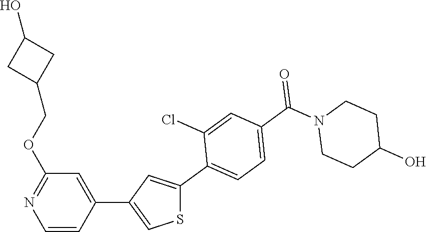 [SMILES: O=C(c1ccc(-c2cc(-c3ccnc(OCC4CC(O)C4)c3)cs2)c(Cl)c1)N1CCC(O)CC1] | | | | | | | +++ | +++ | +++ |
| I-1629118 | | | | | | | | | |
| 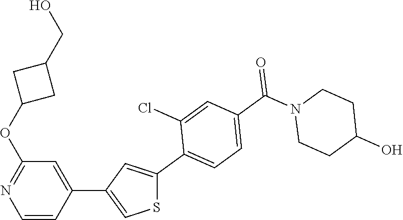 [SMILES: O=C(c1ccc(-c2cc(-c3ccnc(OC4CC(CO)C4)c3)cs2)c(Cl)c1)N1CCC(O)CC1] | | | | | | | +++ | +++ | +++ |
| I-1629110 | | | | | | | | | |
| 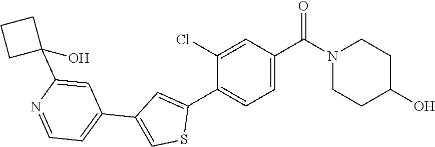 [SMILES: O=C(c1ccc(-c2cc(-c3ccnc(C4(O)CCC4)c3)cs2)c(Cl)c1)N1CCC(O)CC1] | ++ | + | ++ | ++ | +++ | ++ | +++ | +++ | +++ |
| I-1629076 | | | | | | | | | |
| 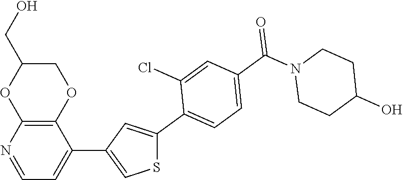 [SMILES: O=C(c1ccc(-c2cc(-c3ccnc4c3OCC(CO)O4)cs2)c(Cl)c1)N1CCC(O)CC1] | | | | | | | | | +++ |
| I-1629181 | | | | | | | | | |

### Biological Example 2: Kinetic Solubility

Kinetic Solubility Procedure: A 10 mM stock solution of a compound is prepared in DMSO, then 4 μL of the stock is added to a deep well plate containing 396 μL of pH 7.4 buffer. The sample plate is vortexed at 800 rpm for 24 h on thermomixer at room temperature. The plate is sealed well during the incubation process. The dimethylsulfoxide (DMSO) content in the sample is 1.0%. The concentration of the evaluated compound in the final incubation is 100 μM. At the end of the incubation period, the sample plate is centrifuged at 4000 rpm for 10 min and analyzed in LC-UV against a calibration curve (CC).

### Biological Example 3: Western Blotting of SREBP Processing

The effect of selected compounds on SREBP processing and activation is evaluated in HepG2 cells via Western blotting. Cells are seeded at a density of 8e⁶ in 150 mm plate in DMEM (Dulbecco's modified Eagle's medium) supplemented with 10% (V/V) heat-inactivated FBS (fetal bovine serum), penicillin G (100 units/ml) and gentamycin (0.2 mg/ml). After overnight incubation, they are washed twice in PBS, and then DMEM media with 0% FBS with 500 nM of compound is added to the plate. Cells are incubated at 37° C. After 48 hours, the cells are washed and lysed to obtain cytoplasmic and nuclear extracts for Western blotting to measure SREBP expression along with topoisomerase I as loading control.

### Biological Example 4: Adipocyte Differentiation and Oil Red-O Staining

The effect of selected compounds on adipocyte differentiation in human pre-adipocyte and 3T3-L1 cells is evaluated.

Human Pre-Adipocyte differentiation: Cells are thawed and seeded at 40,625 cells/cm² in pre-adipocyte media (ZenBio) as per manufacturer's direction. The cells are allowed to reach confluence for 48 hours, and media switched to Adipocyte Differentiation Media (ZenBio) for 7 days. The media is then switched to Adipocyte Maintenance Media (ZenBio) for additional 7 days. The compound being evaluated is added to the cells for day 1-7 during differentiation, or day 7-14 during maturation. Cells are then stained with oil red-O as described below.

NIH 3T3-L1 cell differentiation: Cells are thawed into Pre-Adipocyte Media (ZenBio) and grown to 80-85% confluence. Cells are seeded 50,000 cells/well into 96-wp in Pre-Adipocyte Media (ZenBio) and allowed to reach confluence for 48-72 hours. They are grown an additional 48 hours after reaching confluence, then the media is changed to Differentiation Media (Zen Bio) and incubated for 72 hours. The media is changed to Adipocyte Differentiation Media (ZenBio) using 150 microliters/well in 96-wp for 72 hours, then media is removed and replaced with 150 microliters of Adipocyte Maintenance Media for an additional 8-14 days, feeding cells every 2-3 days. The compound being evaluated is added to the cells for day 3-6 during differentiation, or day 7-14 during maturation. Cells are then stained with oil red-O as described below.

Oil Red-O staining: After maturation, the cells are washed, then fixed in 10% Formalin for 30-60 minutes. The formalin is removed, the cells are washed in water twice, and then the cells are incubated in 60% isopropanol for 5 minutes. The isopropanol is removed and Oil Red-O solution added for 20 minutes with gentle rotation of plate. The stain is removed, the cells washed twice with water, and Hematoxylin added for 1 minute. The cells are washed twice with water and air dried, then images are acquired.

### Biological Example 5: Log D of Compounds

The Log D of selected compounds is evaluated by octanol/aqueous buffer partitioning. 500 μL of organic phase (1-octanol) is added to each well of a 2 mL deep well plate, followed by 500 μL of buffer and 15 μL of test compound in DMSO (0.15 mM). The plate is vortexed for 10 seconds and incubated at room temperature for 1 hr on a plate shaker at 200 rpm. After incubation, the samples are allowed to equilibrate for 20 min and then centrifuged at 4000 rpm for 30 min for complete phase separation. The distribution of test compound in buffer and octanol phase was analyzed by HPLC-UV. Log D=Log (Area of Octanol/Area of Buffer).

### Biological Example 6: In Vivo Activity Assay

The in vivo effect of selected compounds may be assessed using the ob/ob mouse model. The ob/ob mouse is a well characterized model of obesity, fatty liver, and diabetes, which are exhibited due to a mutation in the ob gene, which encodes for leptin.

Compounds are administered by the oral route once or twice daily for 4 weeks in male ob/ob mice. Body weight and food and water intake are assessed daily, and improvements in glucose control are assessed by plasma glucose and insulin measurement. Upon completion of the test period, terminal blood samples are taken and analyzed for triglyceride, cholesterol (total, HDL-C and LDL-C), blood urea nitrogen (BUN), alanine aminotransferase (ALT), and aspartate aminotransferase (AST) levels. Liver and fat pad weights are determined and liver tissue is processed for histological determination of NASH activity scores (NAS: ballooning, inflammation, steatosis and fibrosis). Liver levels of triglycerides, cholesterol, and non-esterified fatty acids (NEFA) are also determined.

### Biological Example 7: Evaluation of In Vivo Pharmacokinetic Properties of Compounds

The in vivo pharmacokinetic properties of compounds by both intravenous and oral administration is evaluated in male Sprague Dawley rats or C57BL/6J mice.

Animals are housed in cages with clean bedding. Certified rodent diet is provided. Water was available ad libitum. Environmental controls for the animal room are set to maintain a temperature of 22° C. to 25° C., humidity of 40-70% RH, and a 12-hour light/12-hour dark cycle. Normal healthy animals certified by the attending veterinarian are selected and acclimatized for minimum three days prior to initiation of study.

Surgical Procedure for Jugular Vein Cannulation of Rats: Rats are anaesthetized with a single dose of ketamine 50 mg/kg i.p.+xylazine 6 mg/kg i.p. The right jugular vein is exposed, a loose ligature is placed caudally, and the cranial end of vein is ligated. A small incision is made between the ligatures into which the catheter (polyethylene 50 tubing of internal diameter 0.58 mm and outer diameter 0.96 mm) is inserted. The catheter is secured in place by tying the loose ligature around the catheterized vessel. A small incision is made in the scapular region to serve as the exit site of the catheter. The catheter is subcutaneously tunneled and exteriorized through scapular incision. A stay suture is placed in the scapular area. Patency is tested, and catheter is filled with a locking solution (heparinized saline) and sealed with a stainless steel plug. The incision is then sutured with sterile suturing material. Anti-septic solution is applied to the sutured site and animal is placed back in the home cage.

To evaluate pharmacokinetic properties of intravenous delivery, male Sprague Dawley rats are administered 2.00 mg compound/kg animal weight through the tail vein. The concentration of the compound in the plasma of the animals is evaluated at 0.083, 0.25, 0.5, 1, 2, 4, 8, 12 and 24 hr by taking blood samples from the cannulated jugular vein.

To evaluate pharmacokinetic properties of oral delivery, rats (male Sprague Dawley rats) or mice (C5Bl/6J) are administered 10 mg compound/kg animal weight by mouth. The concentration of compound in the plasma of the animals is evaluated at 0.25, 0.5, 1, 2, 4, 6, 8, 12 and 24 hr by taking blood samples from the cannulated jugular vein (rats) or through a capillary, guided in retro-orbital plexus (mice).

### Biological Example 8: Evaluation of Compounds on Liver Gene Expression in Mice

The pharmacodynamic properties of selected compounds are evaluated in mice. The animals are housed in cages with clean bedding, and maintained and monitored for good health in accordance with Test Facility SOPs and at the discretion of the laboratory animal veterinarian. Certified rodent diet is provided. Food and water is available ad libitum. Environmental controls for the animal room are set to maintain a temperature of 22° C. to 25° C., humidity of 40-70% RH, and a 12-hour light/12-hour dark cycle. Normal healthy animals certified by the attending veterinarian are selected and acclimatized for minimum three days prior to initiation of study. Animals are identified with body markings.

Aliquots of the compounds being evaluated are weighed and triturated with 0.5% methylcellulose (with the addition of 5% N-methyl pyrrolidone when required to remove clumping) to an appropriate dose concentration. Vials are labeled with the information about study number, test item, concentration and date of preparation. A description of appearance of formulation is recorded (e.g., color, turbidity, etc.). The composition of formulation vehicle is recorded. An aliquot of each dose solution was taken before the dosing began and after dosing is finished, and stored at approximately −20° C. or below for subsequent analysis. The animals are dosed orally through oral gavage needle, and time of dosing is recorded.

After dosing, the mice are anesthetized using gaseous anesthesia. Blood samples are collected through a capillary, guided in retro-orbital plexus, at 6 h or at 24 h. Approximately 100 uL of blood is collected from each mouse, in pre-labeled tubes. The collected blood is stored on ice prior to centrifugation. Blood samples are then centrifuged within 1 hour of collection to separate plasma. Centrifugation was conducted at 2500×g for 15 minutes at 4° C. The plasma is separated and transferred to pre-labeled micro-centrifuge tubes and promptly frozen at −80±10° C. until bioanalysis.

Liver Collection at 6 h or 24 h: Immediately after blood withdrawal for pharmacokinetic evaluation (at 6 or at 24 h), liver tissue is collected without perfusion. Animals are euthanized using carbon dioxide gas in a CO₂ chamber. The whole blood is drained by cutting the both side jugular vein and abdominal aorta. The liver is separated out. All the liver samples are divided in two parts. The first part (200 mg approx.) is snap frozen using liquid nitrogen as soon as possible. These samples are immediately transferred to −80° C. for storage. The remaining part was weighed and used for bioanalysis.

RNA Processing and Gene Expression Analysis: Liver Tissue RNA is harvested with the RNEasy kit and 20-100 ng used to synthesize cDNA with random primers following the manufacturer's protocol. Quantitative PCR was performed on 1 pg to 100 ng cDNA for the following genes: ACACA, ACLY, FASN, LSS, PNPLA3. Gene expression levels are determined using ΔΔCT method comparing treated to vehicle treated samples as a baseline, and fold change calculated. The average value for all 5 genes above is averaged and termed to Total Fold Change.

1. A compound of Formula (II): 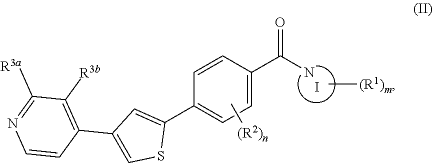
or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
(C₃-C₁₀)cycloalkyl substituted with one or more substituents independently selected from the group consisting of halo and —OR⁵;
(C₃-C₁₀)cycloalkyl substituted with one or more alkyl, wherein the cycloalkyl and each alkyl are independently unsubstituted or substituted with one or more substituents independently selected from the group consisting of halo and —OR⁶;
heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
—OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, heterocycloalkyl, and (C₃-C₁₀)cycloalkyl, and wherein each heterocycloalkyl and cycloalkyl is unsubstituted or substituted with one or more substituents independently selected from the group consisting of alkyl, halo, and —OH;
—CH₂—(C₃-C₁₀)cycloalkyl substituted with one or more substituents independently selected from the group consisting of halo and —OH; or
R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl;
wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

2. A compound of Formula (I): 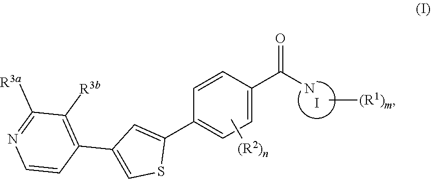
or a pharmaceutically acceptable salt, solvate, tautomer, isotope, or isomer thereof, wherein:
ring I is 3- to 10-membered heterocycloalkyl;
m is an integer from 0 to 8;
each R¹ is independently halo, oxo, alkyl, or —OR⁴, wherein each alkyl is independently unsubstituted or substituted with one or more substituents independently selected from the group consisting of halo and —OH;
n is 0, 1 or 2;
each R² is independently halo, alkyl, or haloalkyl;
R^{3a} is:
(C₃-C₁₀)cycloalkyl substituted with one or more substituents independently selected from the group consisting of halo and —OR⁵;
(C₃-C₁₀)cycloalkyl substituted with one or more alkyl, wherein the cycloalkyl and each alkyl are independently unsubstituted or substituted with one or more substituents independently selected from the group consisting of halo and —OR⁶;
heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH;
—OR⁹, wherein R⁹ is heterocycloalkyl, (C₃-C₁₀)cycloalkyl, or alkyl, wherein the alkyl is unsubstituted or substituted with one or more substituents independently selected from the group consisting of —OH, halo, and (C₃₋₁₀)cycloalkyl, and wherein each heterocycloalkyl and cycloalkyl is unsubstituted or substituted with one or more substituents independently selected from the group consisting of halo and —OH;
—CH₂—(C₃-C₁₀)cycloalkyl substituted with one or more substituents independently selected from the group consisting of halo and —OH; or
R^{3b} is hydrogen, or together with R^{3a} and the atoms to which they are attached forms a non-aromatic heterocyclyl group, wherein the non-aromatic heterocyclyl group is unsubstituted or substituted with one or more substituents independently selected from the group of halo and alkyl;
wherein each alkyl is independently unsubstituted or substituted with one or more substituents independently selected from the group of halo and —OH;
each R⁴, R⁵, R⁶, and R⁷ is independently hydrogen, alkyl, haloalkyl, or —C(O)CHR⁸—NH₂, wherein R⁸ is a (C₁-C₆)alkyl.

3. The compound of claim 1, or a pharmaceutically acceptable salt, solvate, tautomer, isotope, or isomer thereof, wherein ring I is azetidinyl, pyrrolidinyl, or piperidinyl.

4. The compound of claim 1, or a pharmaceutically acceptable salt, solvate, tautomer, isotope, or isomer thereof, wherein ring I is piperidinyl.

5. The compound of claim 1, or a pharmaceutically acceptable salt, solvate, tautomer, isotope, or isomer thereof, wherein m is an integer from 1 to 4.

6. The compound of claim 1, or a pharmaceutically acceptable salt, solvate, tautomer, isotope, or isomer thereof, wherein each R¹ is independently fluoro, —OH, unsubstituted alkyl, or alkyl substituted with one —OH.

7. The compound of claim 1, or a pharmaceutically acceptable salt, solvate, tautomer, isotope, or isomer thereof, wherein n is 1.

8. The compound of claim 7, or a pharmaceutically acceptable salt, solvate, tautomer, isotope, or isomer thereof, wherein R² is chloro.

9. The compound of claim 1, or a pharmaceutically acceptable salt, solvate, tautomer, isotope, or isomer thereof, wherein R^{3a} is (C₃-C₁₀)cycloalkyl substituted with one or more substituents independently selected from the group consisting of halo and —OR⁵.

10. The compound of claim 8, or a pharmaceutically acceptable salt, solvate, tautomer, isotope, or isomer thereof, wherein R^{3a} is (C₃-C₆)cycloalkyl substituted with one or more halo or —OH.

11. The compound of claim 1, or a pharmaceutically acceptable salt, solvate, tautomer, isotope, or isomer thereof, wherein R^{3a} is (C₃-C₁₀)cycloalkyl substituted with one or more alkyl, wherein the cycloalkyl and each alkyl are independently unsubstituted or substituted with one or more substituents independently selected from the group consisting of halo and —OR⁶.

12. The compound of claim 11, or a pharmaceutically acceptable salt, solvate, tautomer, isotope, or isomer thereof, wherein R^{3a} is (C₃-C₆)cycloalkyl substituted with one alkyl, wherein the alkyl is substituted with one —OH; and the cycloalkyl is not further substituted or is further substituted with one or two substituents independently selected from the group consisting of halo and —OR⁷.

13. The compound of claim 1, or a pharmaceutically acceptable salt, solvate, tautomer, isotope, or isomer thereof, wherein R^{3a} is heterocycloalkyl connected through an annular carbon, wherein the heterocycloalkyl is unsubstituted or substituted with one or more substituents independently selected from the group consisting of halo, alkyl, and —OR⁷, wherein each alkyl is unsubstituted or substituted with one or more substituents independently selected from the group consisting of halo and —OH.

14. The compound of claim 13, or a pharmaceutically acceptable salt, solvate, tautomer, isotope, or isomer thereof, wherein the heterocycloalkyl is a 4- to 6-membered heterocycloalkyl comprising one or two heteroatoms.

15. The compound of claim 14, or a pharmaceutically acceptable salt, solvate, tautomer, isotope, or isomer thereof, wherein each heteroatom is oxygen.

16. The compound of claim 13, or a pharmaceutically acceptable salt, solvate, tautomer, isotope, or isomer thereof, wherein the heterocycloalkyl is unsubstituted.

17. The compound of claim 13, or a pharmaceutically acceptable salt, solvate, tautomer, isotope, or isomer thereof, wherein the heterocycloalkyl is substituted with —OH.

18. The compound of claim 1, or a pharmaceutically acceptable salt, solvate, tautomer, isotope, or isomer thereof, wherein R^{3a} is —OR⁹, wherein R⁹ is heterocycloalkyl or (C₃-C₁₀)cycloalkyl, wherein the cycloalkyl is unsubstituted or substituted with one or more substituents independently selected from the group consisting of halo and —OH.

19. The compound of claim 18, or a pharmaceutically acceptable salt, solvate, tautomer, isotope, or isomer thereof, wherein R⁹ is heterocycloalkyl.

20. The compound of claim 18, or a pharmaceutically acceptable salt, solvate, tautomer, isotope, or isomer thereof, wherein R⁹ is (C₃-C₁₀)cycloalkyl.

21. The compound of claim 18, or a pharmaceutically acceptable salt, solvate, tautomer, isotope, or isomer thereof, wherein R⁹ is (C₃-C₁₀)cycloalkyl, wherein the cycloalkyl is substituted with one or more substituents independently selected from the group consisting of halo and —OH.

22. The compound of claim 18, or a pharmaceutically acceptable salt, solvate, tautomer, isotope, or isomer thereof, wherein R⁹ is alkyl, wherein the alkyl is unsubstituted or substituted with one or more substituents independently selected from the group consisting of —OH, halo, and (C₃-C₁₀)cycloalkyl.

23. The compound of claim 1, or a pharmaceutically acceptable salt, solvate, tautomer, isotope, or isomer thereof, wherein R^{3a} is —CH₂—(C₃-C₁₀)cycloalkyl substituted with one or more substituents independently selected from the group consisting of halo and —OH.

24. The compound of claim 1, or a pharmaceutically acceptable salt, solvate, tautomer, isotope, or isomer thereof, wherein R^{3b} together with R^{3a} and the atoms to which they are attached form a non-aromatic heterocyclyl group.

25. The compound of claim 24, or a pharmaceutically acceptable salt, solvate, tautomer, isotope, or isomer thereof, wherein the non-aromatic heterocyclyl group is a 6-membered non-aromatic heterocyclyl group comprising two heteroatoms.

26. The compound of claim 25, or a pharmaceutically acceptable salt, solvate, tautomer, isotope, or isomer thereof, wherein the two heteroatoms are independently O or N.

27. The compound of claim 24, or a pharmaceutically acceptable salt, solvate, tautomer, isotope, or isomer thereof, wherein the non-aromatic heterocyclyl group is unsubstituted.

28. The compound of claim 1, wherein the compound is: 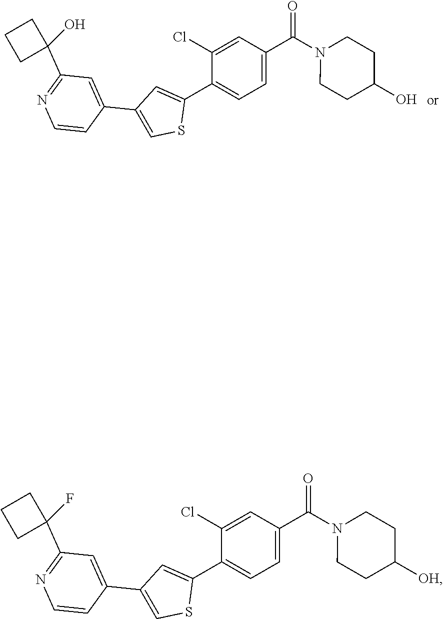 [SMILES: O=C(c1ccc(-c2cc(-c3ccnc(C4(O)CCC4)c3)cs2)c(Cl)c1)N1CCC(O)CC1 ; O=C(c1ccc(-c2cc(-c3ccnc(C4(F)CCC4)c3)cs2)c(Cl)c1)N1CCC(O)CC1]
or a pharmaceutically acceptable salt, solvate, tautomer, isotope, or isomer of any of the foregoing.

29. The compound of claim 1, wherein the compound is: 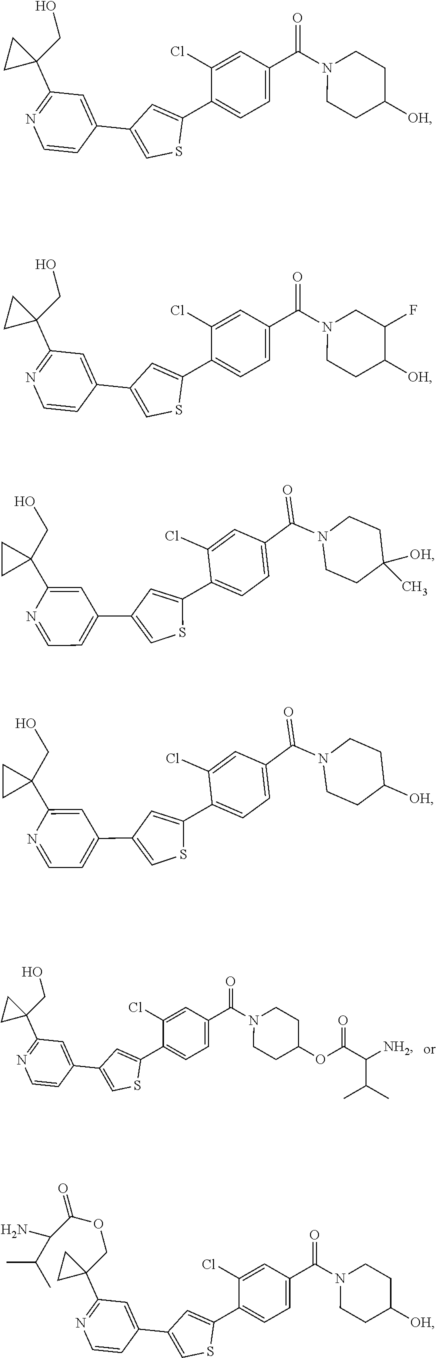
or a pharmaceutically acceptable salt, solvate, tautomer, isotope, or isomer thereof.

30. The compound of claim 1, wherein the compound is: 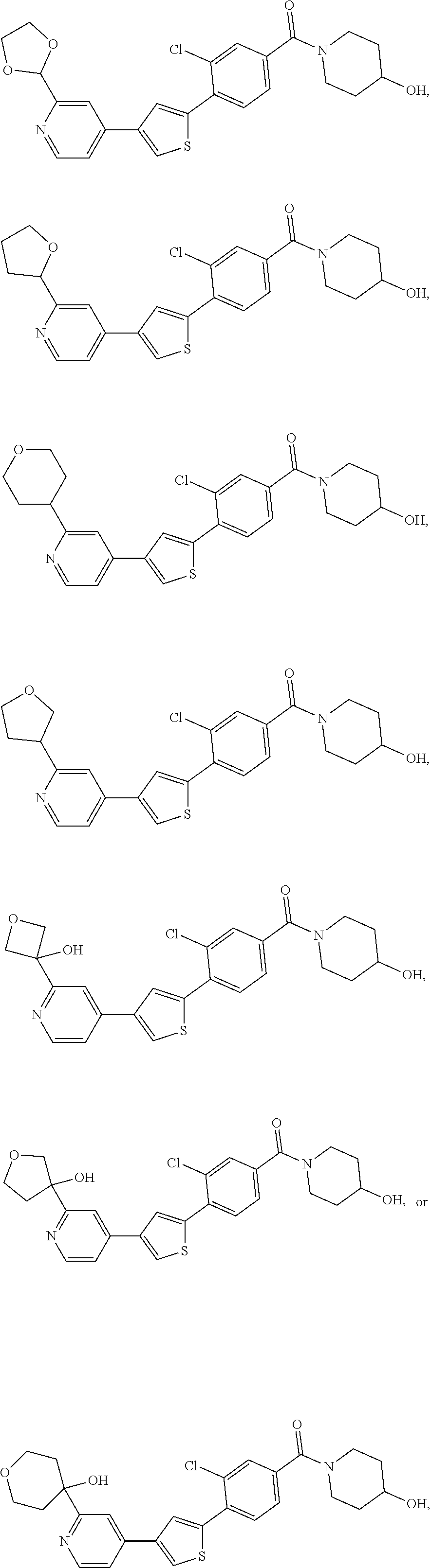
or a pharmaceutically acceptable salt, solvate, tautomer, isotope, or isomer of any of the foregoing.

31. The compound of claim 1, wherein the compound is: 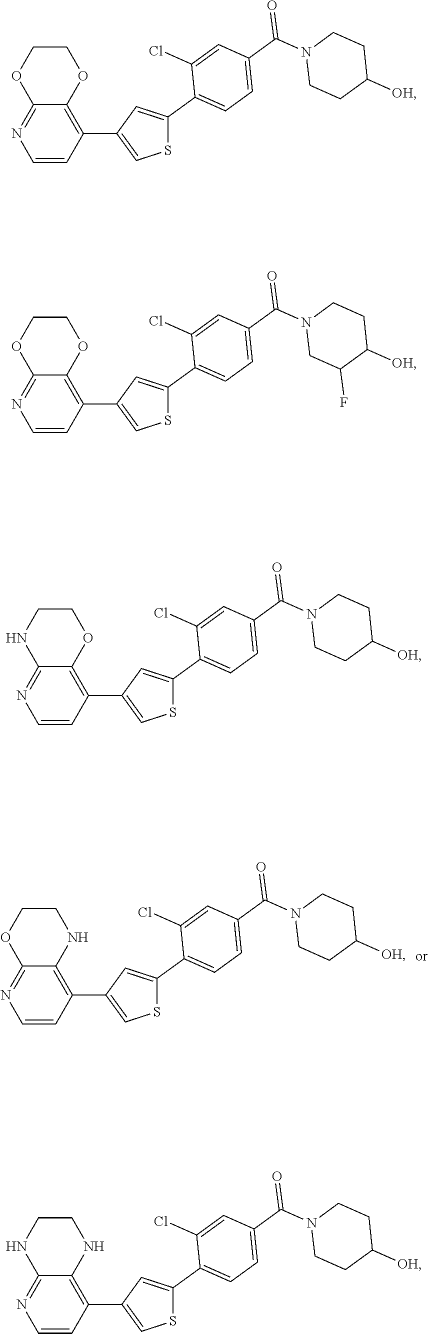 [SMILES: O=C(c1ccc(-c2cc(-c3ccnc4c3OCCO4)cs2)c(Cl)c1)N1CCC(O)CC1 ; O=C(c1ccc(-c2cc(-c3ccnc4c3OCCO4)cs2)c(Cl)c1)N1CCC(O)C(F)C1 ; O=C(c1ccc(-c2cc(-c3ccnc4c3OCCN4)cs2)c(Cl)c1)N1CCC(O)CC1 ; O=C(c1ccc(-c2cc(-c3ccnc4c3NCCO4)cs2)c(Cl)c1)N1CCC(O)CC1 ; O=C(c1ccc(-c2cc(-c3ccnc4c3NCCN4)cs2)c(Cl)c1)N1CCC(O)CC1]
or a pharmaceutically acceptable salt, solvate, tautomer, isotope, or isomer of any of the foregoing.

32. The compound of claim 1, wherein the compound is: 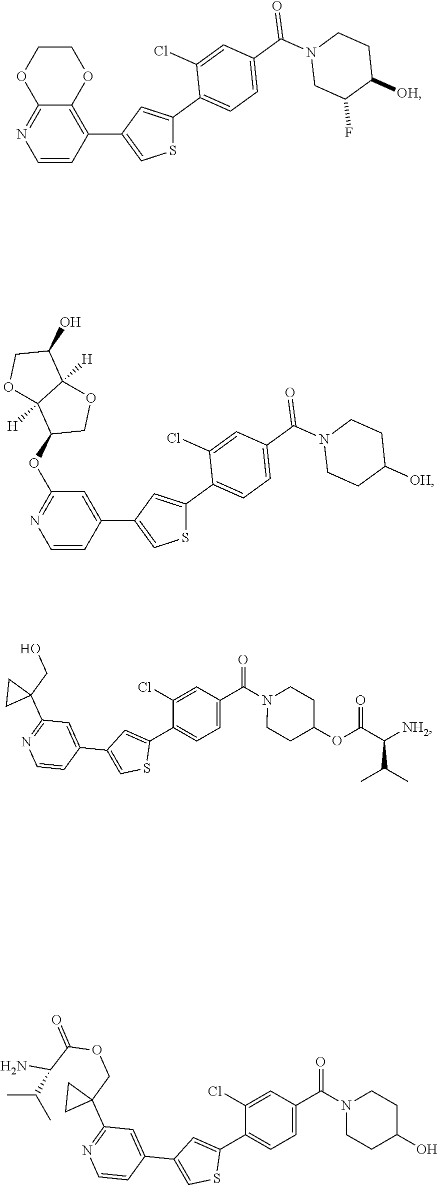
or a pharmaceutically acceptable salt, solvate, tautomer, isotope, or isomer of any of the foregoing.

33. The compound of claim 1, wherein the compound is: 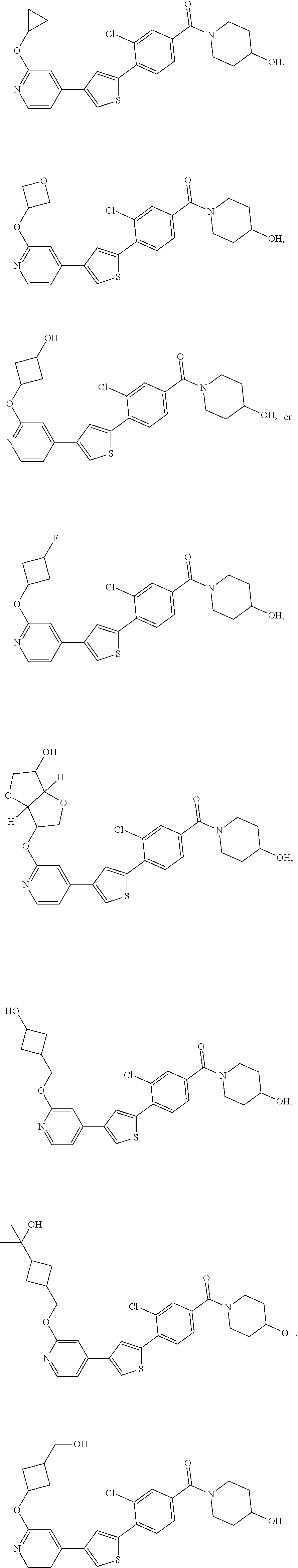
or a pharmaceutically acceptable salt, solvate, tautomer, isotope, or isomer of any of the foregoing.

34. The compound of claim 1, wherein the compound is: 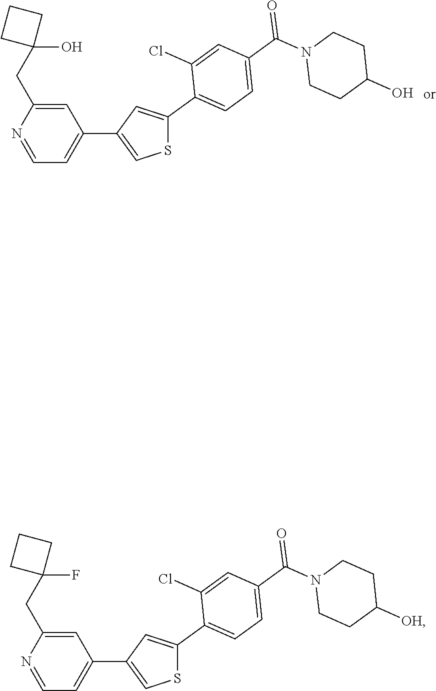 [SMILES: O=C(c1ccc(-c2cc(-c3ccnc(CC4(O)CCC4)c3)cs2)c(Cl)c1)N1CCC(O)CC1 ; O=C(c1ccc(-c2cc(-c3ccnc(CC4(F)CCC4)c3)cs2)c(Cl)c1)N1CCC(O)CC1]
or a pharmaceutically acceptable salt, solvate, tautomer, isotope, or isomer of any of the foregoing.

35. The compound of claim 1, wherein the compound is: 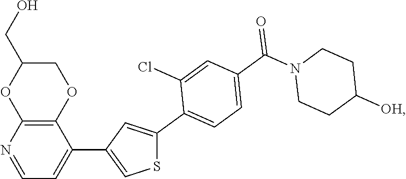 [SMILES: O=C(c1ccc(-c2cc(-c3ccnc4c3OCC(CO)O4)cs2)c(Cl)c1)N1CCC(O)CC1]
or a pharmaceutically acceptable salt, solvate, tautomer, isotope, or isomer of any of the foregoing.

36. The compound of claim 1, wherein the compound is: 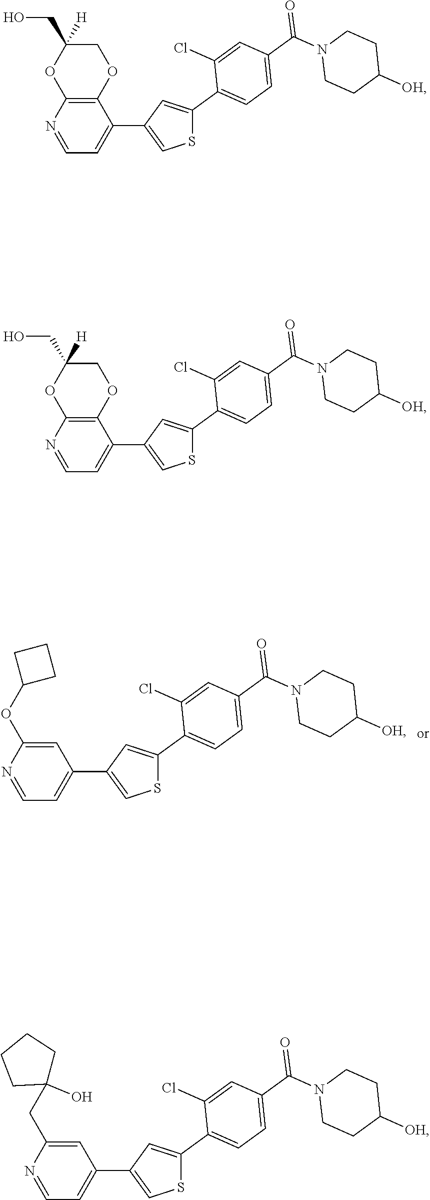 [SMILES: O=C(c1ccc(-c2cc(-c3ccnc4c3OC[C@H](CO)O4)cs2)c(Cl)c1)N1CCC(O)CC1 ; O=C(c1ccc(-c2cc(-c3ccnc4c3OC[C@@H](CO)O4)cs2)c(Cl)c1)N1CCC(O)CC1 ; O=C(c1ccc(-c2cc(-c3ccnc(OC4CCC4)c3)cs2)c(Cl)c1)N1CCC(O)CC1 ; O=C(c1ccc(-c2cc(-c3ccnc(CC4(O)CCCC4)c3)cs2)c(Cl)c1)N1CCC(O)CC1]
or a pharmaceutically acceptable salt, solvate, tautomer, isotope, or isomer of any of the foregoing.

37. The compound of claim 1, wherein the compound is: 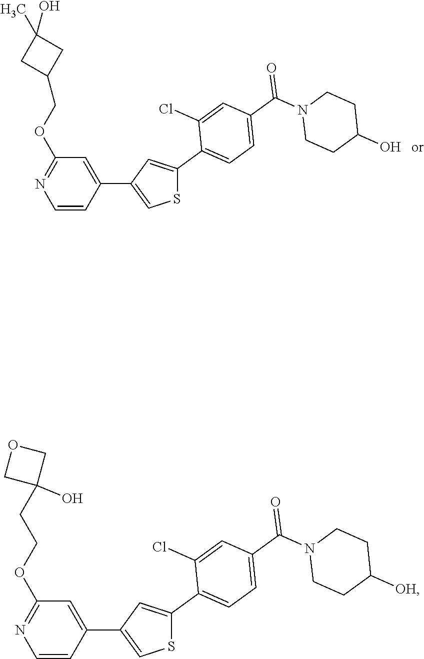 [SMILES: CC1(O)CC(COc2cc(-c3csc(-c4ccc(C(=O)N5CCC(O)CC5)cc4Cl)c3)ccn2)C1 ; O=C(c1ccc(-c2cc(-c3ccnc(OCCC4(O)COC4)c3)cs2)c(Cl)c1)N1CCC(O)CC1]
or a pharmaceutically acceptable salt, solvate, tautomer, isotope, or isomer of any of the foregoing.

38. A pharmaceutical composition, comprising a compound of claim 1, or a pharmaceutically acceptable salt, solvate, tautomer, isotope, or isomer thereof, and a pharmaceutically acceptable excipient.

39. A method of inhibiting a sterol regulatory element-binding protein (SREBP), comprising contacting the SREBP or contacting an SREBP cleavage activating-protein (SCAP) with an effective amount of a compound of claim 1, or a pharmaceutically acceptable salt, solvate, tautomer, isotope, or isomer thereof.

40. A method of inhibiting the proteolytic activation of a sterol regulatory element-binding protein (SREBP), comprising contacting an SREBP cleavage activating-protein (SCAP) with an effective amount of a compound of claim 1, or a pharmaceutically acceptable salt, solvate, tautomer, isotope, or isomer thereof.

41. A method of treating a disorder in a subject in need thereof, comprising administering to the subject in need thereof an effective amount of a compound of claim 1, or a pharmaceutically acceptable salt, solvate, tautomer, isotope, or isomer thereof.

42. A method of treating a disorder in a subject in need thereof, wherein the disorder is mediated by a sterol regulatory element-binding protein (SREBP), comprising administering to the subject in need thereof an effective amount of a compound of claim 1, or a pharmaceutically acceptable salt, solvate, tautomer, isotope, or isomer thereof.

43.-50. (canceled)
